SEQUENCE LISTING (57 sequences)

SEQ ID NO: 1 - protein, 1226 aa, synthetic construct
  REGION 1..1226: note = CasM.21544
MKNFFQLFTNQYELSKTLRFELRPIENTAELLKKNKIFEKDKKVYEYYQKTKKYFDKLHCEFIDEALVNTSLPNKDYSIYDKLFFEYKKDKENKKIYKALNEHAKKLRTNIVKDFEKTAEKWRDEYILSIKDEKSQKKLKQLEGLDLLFKTEVFDFLKKKYPEAQIEGNSIFDSFNKFFTYFDNFHQSRKNFYKDDGTASAIPTRIIDDNLPKFLENKKIFEEEYKVLNLSREEKDLFRLDFYNKCFTQTGIENYNEIVSKINSKVNKYRQKNKKSKIHFMKELFKQILSKRSKQATEQDTSISIENDSQVFEVLQDFIMETKNCNRMAKNILETFIQHQEEFDLQKVFLKGSNVTRISDMWFTSWFAFGDLLPKNSTGKKLANFISFQDIKNALEKTEIQDVFKKKYAKYNKESFYDQFLQIFDYEFHKALNDCEKNIVEAEKMMKVDKVYSNKKGILRNGKNGEKQKEIIKDFADSALSIYQMMKYFALEKGKKPVTDMDEDPRFYNDYKDWCEKSRTLLYYNEFRNYLTKKPYAEDKIKLNFEKSTLLMGWSANEEGNLQYYSSILRKDNKYYLGLMSSASTFNINKKEAFETRDGFYELMNYRQLKSTTIYGSLYKGEYKKEYRKDKETLTNQELIERTKKILEKQKFSYPQLEVILDKKYKAAEDLAKDLGNIVLYNIGFVKISKRYVNNLTNENFYLFEISNKDFLNKKKKGNVNLHTKYFQLLFDKKNLESKKGAILKLSGGGEVFYRKATKNLPKKKDKKGKEIIDRKRYAEDKILFHLPIVLNIGFKDKQINSKVNQVLATQRKARIIGIDRGEKHLLYISVIDENANILKIKSLNTIDVPNKKEPDDYHKLLDEKEKERDDERKSWHTIENIKELKHGYISQVVNEIDKIIFECLDERILPIIVFEDLNIGFKRGRFKIEKQVYQKFELALAKKLNYLVSKEKGNYLNAFQFTPPVNNFQDIHKKQVGIIFYIPASFTSAICPVCGFRKRLYGFTFKNINQVKKLLAEHEFNIFYDGKRFNFECLASKENEKKENKNGLYTEIFLNKKLFFNSDIERLFNKKSKDNKKWETISFIATEELKKLFEGKIDLKRNIMDQILAGDFRAEFYQQLIFIINRILNLRNCHAVEHRDFIACPSCGFHSEKNYEKLKSRYAGKEKFEFNGDANGAYNIARKGILLIKKIRQFAKSNNIEKLMPYDHLQIDMQEWDKFCAKNKS

SEQ ID NO: 2 - protein, 1281 aa, synthetic construct
  REGION 1..1281: note = CasM.21526
MLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 3 - protein, 1273 aa, synthetic construct
  REGION 1..1273: note = CasM.21550
MQDKTGWSSFTNKYSLSKTLRFELKPVGNTQKMLEDDGVFQKDRERQENYKKVKPFMDKLHREFIKEALNNLKLEGLTEYFEIFKKFRKDKNNKELKNAEKKLRQIIGRCYTETAQIWVEKYKEFGFKKKNIGFLFEEGVFELMKLKYGNDEASQIEKNGEVLSIFDGWKGFLGYFKKFFETRNNFYKDDGTSTAVSTRIINENLKIYLDNLIKYNKIKDKVDFKEADILQENKLNLSDFFNVESYAKYSLQKGIDYYNEILGGKTLKNGTKLKGLNEVINEYKQKNKSGELSKFKMLKKQILGEGEDRTLFEEIENEDELKDVLKDFFYNADPKITLFKTLLEDFFSNTEKYKDELDKIYFNTVAINGILHRWVDDSGVFQKYLFEVLKSNKLVKSNHYDKKEDSYKFPDFISFEHIKVALENCERDGLKDKFWKEKYYTKECLTENGLANLWQEFLEIYKCEFKKLYDYKTDDNDCYLQYRDNYKKYILDANFNPKEKSAKDIIKDYLDSVLSIYQLAKYFALEKKKVWTTDYETGDFYYEYIKFYEDTYEQIIKPYNLVRNYLTRKPINTAKKWKLNFDNAYLASGWDKDKEVSNLTVILRRDEQYYLAIMKKGKNKIFEKKFSCGEFEKMEYKQIAEASSDIHNLVLMNDGSCRRCIKMHDKRKYWPLDISIIKEKKSYAKENFVRRDFERFVNYMKKCSLLYWKEYDLKFSDTSTYKNINDFTNEIASQGYKLSFSAIPESYINEKNNNGELYLFQIYNKDFGIKTEGNKNLHTMYWESIFSEENRFRNFIVKLNGKAEIFYRPKSEQVEKEQRNFTREIIKNRRYTENKIYFHCPITLNRISRENVKKFNNGINNYIATNPNINILGVDRGEKHLVYYAIVDQDGKLIDAEDATGSFNTIGSTDYHRLLEEKAKDREKERKDWDLIRGIKDLKKGYISLVVRKIADLAIKYNAIIIFEDLNTRFKQIRGGMEKSVYQQLEKALINKLSFLVNKGEKDPEQAGHLLKAYQLAAPFQTFDKMGRQTGIIFYTQASYTSKIDPITGWRPNLYLKYRNIDDSKESIKKFKSILFNKEKNRFEFTYDLKDFVDFEEDKIPEKTEWTLCSSVERHKWNRHMNNNKGGYEVYKDLTENFYKLFDENNISMNKDIVDQVESISNGNFFRQFIYLFNLVCQIRNTDEKAEDVDKRDFILSPVEPFFDSRRAKDFKAYGDNLPKNGDENGAYNIARKGVLIIKKIKEYYNQNGSCDKLGWGDLSISHKEWDDFATNN

SEQ ID NO: 4 - protein, 1467 aa, synthetic construct
  REGION 1..1467: note = CasM.21530
MLIKDFTNLYSLNKTLRFELVPQGKTNDYIDKWIKELELENKHEVLKDKVDETIAKYIENIKDKLNESNYEPETKSKIIQCIENSKSKINYTDLKENSVREVVSIWFKEINNELNEVDLRKNIKNKINNRIKKLSEIVEDELKNIILEDKNRAKKYEKVKKILDEYHKDFIERVLKNLDQTDFNKLLKEYLDLYTKKKDKKETKEFVKIKKNLRKKISDTLTKNPDYKILDKDKLFKRGKKSEKKDSSHEENDDTNVGGILEKFLEENSTLKHKLEESLRNNEDETIDLFELLRSFEGFTTYFRNFHKNRENLYSDEDKFSTIAHRLIHENLPKFIDNIKIYQKAKEKGLPIEEIQKQLGITESLDDVFSIEYFTKCLTQSGIDRYNYILGGKSVENGQNIRGVNSFINNEINQSVSEKKDRVPVMKMLYKLPLFDRTSSSFRYDPIENDQDLIERIVSFYERNLTQYIDTTTSDDTPVNILEMIKELLQNIHNYREGLYVNGGTTLTQISQKIFGDWRYIHDVLSYYYDTIISPLKKDKKGNLKSRLGTEEKQKEKWMKQKQFSVVLIEKALNEYKKVETNEELKNKITDTTLCDFFARCGTDKDGKDLFKRIDEKLTEKNSHSESLKNLMNFNFGNERKLMQNESRISLIKNFLDAILGDKEDITAGLLHFIKPLIPREEVADKNELFYSEFERYYNLLSEIIPLYNKVRNYLTQKPFSIEKIKLNFENPMLLAGWDVNKEADNSCVLFRKNGFYYLGIMNKYYRDVFKNYETANDGEDFYEKMVYKLLPGPNKMLPKVFFSEKNIGYFNPSDEILRIRNTASYSKNGKPREGYKKADFSVNDCQQLIDFFKESILKHEDWSKFNFQFKPTSRYNDISEFYKDVKNQGYKINFVKIPSTYINQLVQEGKLYLFKIHNKDFSEKKKPGGKDNLHTLYWKMLFSEENLKDVVFKLDGEAEIFFRPKSIIYDENIWKNGHHNEKLKDKFNYPIIKDRRFALDKIYFHVPITINFKAGDVPDNEDEEKNNQQNEMKNESFNQKILSFLKDRDDVHIIGIDRGERHLLYVVVINSDGKIVEQFSLNKIPREKSDVPIDYLDRLNNKEGQLDEARKNWQTIQNIKELKEGYLSHVIHIITRLIVKYNAIVVMEDLNSGFKRGRQKVEKQVYQKFEKMLINKLNYLVFKDKKAGEPGGVLKGLQLTDKFESFKNLGKQSGIIFYVPPAYTSAIDPVTGYVQYLYPLKRADSIEKAKNFYSKFDSTKYNAEKDRFEFTFDYEKIPNTRYEGKSQWTICTSDEERYYWDKSLNNGKGGQKEYKTTSEMKQLFKEAAIPYHTGEELKQYIAGISGNPEKKIKDKEFFNRLNLLLHVTLKLRHNNGKDGDKEQDYILSPVEPFFDSRKAKGDLPENADANGAFNIARKGLVLLKRLKKMDIDEFEKTKKSKDGKSQWLPHKEWLEFSQEKHAAVLN

SEQ ID NO: 5 - protein, 18 aa, synthetic construct
  REGION 1..18: note = nuclear localization signal
KRPAATKKAGQAKKKKEF

SEQ ID NO: 6 - protein, 20 aa, synthetic construct
  REGION 1..20: note = endosomal escape peptide
  VARIANT 4: note = X is lysine, histidine, or arginine
  VARIANT 8: note = X is lysine, histidine, or arginine
  VARIANT 11: note = X is lysine, histidine, or arginine
  VARIANT 15: note = X is lysine, histidine, or arginine
  VARIANT 19: note = X is lysine, histidine, or arginine
GLFXALLXLLXSLWXLLLXA

SEQ ID NO: 7 - protein, 20 aa, synthetic construct
  REGION 1..20: note = endosomal escape peptide
GLFHALLHLLHSLWHLLLHA

SEQ ID NO: 8 - protein, 10 aa, synthetic construct
  REGION 1..10: note = linker
  VARIANT 2..10: note = individual amino acid residues may be present or absent
GGGGGGGGGG

SEQ ID NO: 9 - protein, 20 aa, synthetic construct
  REGION 1..20: note = linker
  VARIANT 3..4: note = grouped amino acid residues may be present or absent
  VARIANT 5..6: note = grouped amino acid residues may be present or absent
  VARIANT 7..8: note = grouped amino acid residues may be present or absent
  VARIANT 9..10: note = grouped amino acid residues may be present or absent
  VARIANT 11..12: note = grouped amino acid residues may be present or absent
  VARIANT 13..14: note = grouped amino acid residues may be present or absent
  VARIANT 15..16: note = grouped amino acid residues may be present or absent
  VARIANT 17..18: note = grouped amino acid residues may be present or absent
  VARIANT 19..20: note = grouped amino acid residues may be present or absent
GSGSGSGSGSGSGSGSGSGS

SEQ ID NO: 10 - protein, 50 aa, synthetic construct
  REGION 1..50: note = linker
  VARIANT 6..10: note = grouped amino acid residues may be present or absent
  VARIANT 11..15: note = grouped amino acid residues may be present or absent
  VARIANT 16..20: note = grouped amino acid residues may be present or absent
  VARIANT 21..25: note = grouped amino acid residues may be present or absent
  VARIANT 26..30: note = grouped amino acid residues may be present or absent
  VARIANT 31..35: note = grouped amino acid residues may be present or absent
  VARIANT 36..40: note = grouped amino acid residues may be present or absent
  VARIANT 41..45: note = grouped amino acid residues may be present or absent
  VARIANT 46..50: note = grouped amino acid residues may be present or absent
GSGGSGSGGSGSGGSGSGGSGSGGSGSGGSGSGGSGSGGSGSGGSGSGGS

SEQ ID NO: 11 - protein, 60 aa, synthetic construct
  REGION 1..60: note = linker
  VARIANT 7..12: note = grouped amino acid residues may be present or absent
  VARIANT 13..18: note = grouped amino acid residues may be present or absent
  VARIANT 19..24: note = grouped amino acid residues may be present or absent
  VARIANT 25..30: note = grouped amino acid residues may be present or absent
  VARIANT 31..36: note = grouped amino acid residues may be present or absent
  VARIANT 37..42: note = grouped amino acid residues may be present or absent
  VARIANT 43..48: note = grouped amino acid residues may be present or absent
  VARIANT 49..54: note = grouped amino acid residues may be present or absent
  VARIANT 55..60: note = grouped amino acid residues may be present or absent
GGSGGSGGSGGSGGSGGSGGSGGSGGSGGSGGSGGSGGSGGSGGSGGSGGSGGSGGSGGS

SEQ ID NO: 12 - protein, 40 aa, synthetic construct
  REGION 1..40: note = linker
  VARIANT 5..8: note = grouped amino acid residues may be present or absent
  VARIANT 9..12: note = grouped amino acid residues may be present or absent
  VARIANT 13..16: note = grouped amino acid residues may be present or absent
  VARIANT 17..20: note = grouped amino acid residues may be present or absent
  VARIANT 21..24: note = grouped amino acid residues may be present or absent
  VARIANT 25..28: note = grouped amino acid residues may be present or absent
  VARIANT 29..32: note = grouped amino acid residues may be present or absent
  VARIANT 33..36: note = grouped amino acid residues may be present or absent
  VARIANT 37..40: note = grouped amino acid residues may be present or absent
GGGSGGGSGGGSGGGSGGGSGGGSGGGSGGGSGGGSGGGS

SEQ ID NO: 13 - protein, 4 aa, synthetic construct
  REGION 1..4: note = linker
GGSG

SEQ ID NO: 14 - protein, 5 aa, synthetic construct
  REGION 1..5: note = linker
GGSGG

SEQ ID NO: 15 - protein, 5 aa, synthetic construct
  REGION 1..5: note = linker
GSGSG

SEQ ID NO: 16 - protein, 5 aa, synthetic construct
  REGION 1..5: note = linker
GSGGG

SEQ ID NO: 17 - protein, 5 aa, synthetic construct
  REGION 1..5: note = linker
GGGSG

SEQ ID NO: 18 - protein, 5 aa, synthetic construct
  REGION 1..5: note = linker
GSSSG

SEQ ID NO: 19 - RNA, 40 nt, synthetic construct
  misc_feature 1..40: note = crRNA
  source 1..40: mol_type = other RNA
agatttctacttttgtagattattaaatactcgtattgct

SEQ ID NO: 20 - RNA, 40 nt, synthetic construct
  misc_feature 1..40: note = crRNA
  source 1..40: mol_type = other RNA
gaatttctactattgtagattattaaatactcgtattgct

SEQ ID NO: 21 - RNA, 40 nt, synthetic construct
  misc_feature 1..40: note = crRNA
  source 1..40: mol_type = other RNA
agatttctactattgtagattattaaatactcgtattgct

SEQ ID NO: 22-23 - skipped (2 entries)

SEQ ID NO: 24 - RNA, 10 nt, synthetic construct
  misc_feature 1..10: note = ss nucleic acid in a reporter
  source 1..10: mol_type = other RNA
tttttttttt

SEQ ID NO: 25 - DNA, 10 bp, synthetic construct
  misc_feature 1..10: note = ss nucleic acid in a reporter
  misc_feature 1..4: note = DNA fragment
  misc_feature 5..6: note = RNA fragment
  misc_feature 7..10: note = DNA fragment
  source 1..10: mol_type = other DNA
tttttttttt

SEQ ID NO: 26-32 - skipped (7 entries)

SEQ ID NO: 33 - DNA, 10 bp, synthetic construct
  misc_feature 1..10: note = single stranded nucleic acid in a reporter
  source 1..10: mol_type = other DNA
tttttttttt

SEQ ID NO: 34 - DNA, 12 bp, synthetic construct
  misc_feature 1..12: note = single stranded nucleic acid in a reporter
  source 1..12: mol_type = other DNA
tttttttttttt

SEQ ID NO: 35 - DNA, 14 bp, synthetic construct
  misc_feature 1..14: note = single stranded nucleic acid in a reporter
  source 1..14: mol_type = other DNA
tttttttttttttt

SEQ ID NO: 36-39 - skipped (4 entries)

SEQ ID NO: 40 - protein, 1224 aa, synthetic construct
  REGION 1..1224: note = Cas12 variant
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 41 - protein, 529 aa, synthetic construct
  REGION 1..529: note = Cas14a.1
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 42-46 - skipped (5 entries)

SEQ ID NO: 47 - protein, 4 aa, unidentified
  REGION 1..4: note = N-terminal sequence
MEEK

SEQ ID NO: 48 - protein, 1159 aa, synthetic construct
  REGION 1..1159: note = CasM26
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 49 - DNA, 20 bp, synthetic construct
  misc_feature 1..20: note = ss nucleic acid in a reporter
  source 1..20: mol_type = other DNA
tttttttttttttttttttt

SEQ ID NO: 50 - DNA, 12 bp, synthetic construct
  misc_feature 1..12: note = ss nucleic acid in a reporter
  source 1..12: mol_type = other DNA
cccccccccccc

SEQ ID NO: 51 - RNA, 181 nt, synthetic construct
  misc_feature 1..181: note = sgRNA
  source 1..181: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttgaaagaatgaaggaatgcaaccccccagcgcttcagcgttc

SEQ ID NO: 52 - RNA, 40 nt, synthetic construct
  misc_feature 1..40: note = crRNA
  source 1..40: mol_type = other RNA
gaatttctactattgtagatgccgataatgatgtagggat

SEQ ID NO: 53 - RNA, 41 nt, synthetic construct
  misc_feature 1..41: note = crRNA
  source 1..41: mol_type = other RNA
taatttctactaagtgtagatgccgataatgatgtagggat

SEQ ID NO: 54 - RNA, 41 nt, synthetic construct
  misc_feature 1..41: note = crRNA
  source 1..41: mol_type = other RNA
taatttctactaagtgtagatcccccagcgcttcagcgttc

SEQ ID NO: 55 - RNA, 20 nt, synthetic construct
  misc_feature 1..20: note = guide nucleic acid
  source 1..20: mol_type = other RNA
gaatttctactattgtagat

SEQ ID NO: 56 - RNA, 21 nt, synthetic construct
  misc_feature 1..21: note = guide nucleic acid
  source 1..21: mol_type = other RNA
taatttctactaagtgtagat

SEQ ID NO: 57 - protein, 4 aa, unidentified
  REGION 1..4: note = N-terminal sequence
MLKS

(19) United States
(12) Patent Application Publication
(10) Pub. No.: US 2024/0327810 A1
(43) Pub. Date: Oct. 3, 2024

(54) EFFECTOR PROTEINS AND METHODS OF USE

(71) Applicant: Mammoth Biosciences, Inc., Brisbane, CA (US)

(72) Inventors: James Paul BROUGHTON, South San Francisco, CA (US); Janice Sha Chen, San Francisco, CA (US); Jesus Ching, Saratoga, CA (US); Clare Louise Fasching, Redwood City, CA (US); Lucas Benjamin Harrington, San Francisco, CA (US); Lior Kreindler, New York, NY (US); Bridget Ann Paine Mckay, Portola Valley, CA (US); David Paez-Espino, Concord, CA (US); Benjamin Julius Rauch, Oakland, CA (US); Matthew Verosloff, South San Francisco, CA (US); William Douglass Wright, Fairfield, CA (US)

(21) Appl. No.: 18/501,102

(22) Filed: Nov. 3, 2023

Related U.S. Application Data
(63) Continuation of application No. PCT/US22/28865, filed on May 11, 2022.
(60) Provisional application No. 63/187,298, filed on May 11, 2021.
(60) Provisional application No. 63/229,951, filed on Aug. 5, 2021.

(51) Int. Cl. C12N 9/22; C12N 15/11; C12Q 1/6876

(52) U.S. Cl. CPC C12N 9/22; C12N 15/111; C12Q 1/6876; C12N 2310/20

(57) ABSTRACT

The present disclosure provides compositions of effector proteins. The compositions may comprise engineered guide nucleic acids. Also disclosed are the methods and systems for detecting and modifying target nucleic acids using the same. The compositions may provide nucleic acid modification and/or detection at relatively high temperatures.

### CROSS-REFERENCE TO RELATED APPLICATIONS

This application is a continuation of International Application No. PCT/US2022/028865, filed May 11, 2022, which claims priority to U.S. Provisional Patent Application No. 63/229,951, filed Aug. 5, 2021, and U.S. Provisional Patent Application No. 63/187,298, filed May 11, 2021, each of which is incorporated by reference herein in its entirety for all purposes.

### REFERENCE TO AN ELECTRONIC SEQUENCE LISTING

The contents of the electronic sequence listing (MABI_017_01US_SeqList_ST26.xml; Size: 80,087 bytes; and Date of Creation: Nov. 2, 2023) are herein incorporated by reference in their entirety.

### TECHNICAL FIELD

The present disclosure relates to programmable nucleases and uses of such nucleases for detecting and/or modifying target nucleic acids.

### BACKGROUND

Programmable nucleases are proteins that bind and cleave nucleic acids in a sequence-specific manner. A programmable nuclease may bind a target region of a nucleic acid and cleave the nucleic acid within the target region or at a position adjacent to the target region. In some instances, a programmable nuclease is activated when it binds a target region of a nucleic acid to cleave regions of the nucleic acid that are near, but not adjacent to the target region. A programmable nuclease, such as a CRISPR-associated (Cas) protein, may be coupled to an engineered guide nucleic acid that imparts activity or sequence selectivity to the programmable nuclease. In general, guide nucleic acids comprise a CRISPR RNA (crRNA) that is at least partially complementary to a target nucleic acid. In some cases, guide nucleic acids comprise a trans-activating crRNA (tracrRNA), at least a portion of which interacts with the programmable nuclease. In some cases, a tracrRNA or intermediary RNA is provided separately from the engineered guide nucleic acid. The tracrRNA may hybridize to a portion of the engineered guide nucleic acid that does not hybridize to the target nucleic acid.

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid that is hybridized to a guide RNA (crRNA or single guide RNA (sgRNA)), wherein cleavage occurs within or directly adjacent to the region of the target nucleic acid that is hybridized to guide RNA. Trans cleavage activity (also referred to as transcollateral cleavage) is cleavage of ssDNA or ssRNA that is near, but not hybridized to the guide RNA. Trans cleavage activity is triggered by the hybridization of guide RNA to the target nucleic acid. Nickase activity is the selective cleavage of one strand of a dsDNA molecule. While certain programmable nucleases may be used to edit and detect nucleic acid molecules in a sequence specific manner, challenging biological sample conditions (e.g., high viscosity, metal chelating) may limit their accuracy and effectiveness. There is thus a need for systems and methods that employ programmable nucleases having specificity and efficiency across a wide range of sample conditions.

### SUMMARY

The present disclosure provides for compositions and systems comprising a CRISPR/Cas protein, and uses thereof. Compositions, systems, and methods of the present disclosure are useful for the modification and detection of nucleic acids. In some instances, compositions are capable of cleaving nucleic acids at high temperatures, e.g., 45° C., 50° C., 55° C., 60° C., or 65° C., making them especially suitable for diagnostic devices and applications.

Disclosed herein, are compositions. In an aspect, a composition comprises an effector protein and an engineered guide nucleic acid, wherein the effector protein comprises an amino acid sequence that is at least 75%, at least 80%, at least 85%, at least 90%, at least 95%, at least 98%, at least 99%, or 100% identical to any one of SEQ IDs NO: 1-4.

Disclosed herein, are compositions. In an aspect, a composition comprises an effector protein that comprises an amino acid sequence that is at least 75%, at least 80%, at least 85%, at least 90%, at least 95%, at least 98%, at least 99%, or 100% identical to SEQ ID NO: 1.

Disclosed herein, are compositions. In an aspect, a composition comprises an effector protein that comprises an amino acid sequence that is at least 75%, at least 80%, at least 85%, at least 90%, at least 95%, at least 98%, at least 99%, or 100% identical to SEQ ID NO: 2.

Disclosed herein, are compositions. In an aspect, a composition comprises an effector protein that comprises an amino acid sequence that is at least 75%, at least 80%, at least 85%, at least 90%, at least 95%, at least 98%, at least 99%, or 100% identical to SEQ ID NO: 3.

Disclosed herein, are compositions. In an aspect, a composition comprises an effector protein that comprises an amino acid sequence that is at least 75%, at least 80%, at least 85%, at least 90%, at least 95%, at least 98%, at least 99%, or 100% identical to SEQ ID NO: 4.

Disclosed herein, are compositions. In an aspect, a composition comprises an engineered guide nucleic acid, wherein the engineered guide nucleic acid comprises a clustered regularly interspaced short palindromic repeats RNA (crRNA) comprising a nucleobase sequence that is at least 75%, at least 80%, at least 85%, at least 90%, at least 95%, at least 98%, at least 99%, or 100% identical to any one of SEQ ID NOs: 19-21.

Disclosed herein, are compositions. In an aspect, a composition comprises an engineered guide nucleic acid, wherein the engineered guide nucleic acid comprises a clustered regularly interspaced short palindromic repeats RNA (crRNA) comprising a nucleobase sequence that is at least 75%, at least 80%, at least 85%, at least 90%, at least 95%, at least 98%, at least 99%, or 100% identical to SEQ ID NO: 19.

Disclosed herein, are compositions. In an aspect, a composition comprises an engineered guide nucleic acid, wherein the engineered guide nucleic acid comprises a clustered regularly interspaced short palindromic repeats RNA (crRNA) comprising a nucleobase sequence that is at least 75%, at least 80%, at least 85%, at least 90%, at least 95%, at least 98%, at least 99%, or 100% identical to SEQ ID NO: 20.

Disclosed herein, are compositions. In an aspect, a composition comprises an engineered guide nucleic acid, wherein the engineered guide nucleic acid comprises a clustered regularly interspaced short palindromic repeats RNA (crRNA) comprising a nucleobase sequence that is at least 75%, at least 80%, at least 85%, at least 90%, at least 95%, at least 98%, at least 99%, or 100% identical to SEQ ID NO: 21.

Disclosed herein, are compositions. In an aspect, a composition comprises an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 1, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 19.

Disclosed herein, are compositions. In an aspect, a composition comprises an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 2, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 20.

Disclosed herein, are compositions. In an aspect, a composition comprises an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 3, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 19.

Disclosed herein, are compositions. In an aspect, a composition comprises an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 4, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 21.

In some embodiments, the engineered guide nucleic acid further comprises a trans-activating crRNA (tracrRNA). In some embodiments, the engineered guide nucleic acid is a single guide RNA (sgRNA).

Disclosed herein, are systems for detecting a target nucleic acid. In an aspect, a system for detecting a target nucleic acid comprises any compositions described thereof in a solution, wherein the solution comprises at least one of a buffering agent, a salt, a crowding agent, a detergent, a reducing agent, a competitor, and a detection agent.

In some embodiments, the pH of the solution is selected from at least about 6.0, at least about 6.5, at least about 7.0, at least about 7.5, at least about 8.0, at least about 8.5, or at least about 9. In some embodiments, the pH of the solution ranges from at least about 6.0, at least about 6.5, at least about 7.0, at least about 7.5, at least about 8.0, at least about 8.5, or at least about 9 to about 10, about 11, or about 12. In some embodiments, the pH of the solution is from about 6 to about 12. In some embodiments, the salt is selected from the group consisting of a magnesium salt, a potassium salt, a sodium salt, and a calcium salt. In some embodiments, the concentration of the salt in the solution is selected from at least about 1 millimolar (mM), at least about 3 mM, at least about 5 mM, at least about 7 mM, at least about 9 mM, at least about 11 mM, at least about 13 mM, or at least about 15 mM.

In some embodiments, the detection reagent is selected from a reporter nucleic acid, a detection moiety, an additional effector protein, or a combination thereof. In some embodiments, the detection reagent is the reporter nucleic acid. In some embodiments, the reporter nucleic acid comprises a fluorophore, a quencher, or a combination thereof. In some embodiments, the reporter nucleic acid comprises the fluorophore. In some embodiments, the reporter nucleic acid comprises the quencher. In some embodiments, the reporter nucleic acid is in the form of single stranded deoxyribonucleic acid (DNA). In some embodiments, the system comprises at least one amplification reagent for amplifying the target nucleic acid. In some embodiments, the at least one amplification reagent is selected from the group consisting of a primer, an activator, a deoxynucleoside triphosphate (dNTP), a ribonucleoside triphosphate (rNTP), and combinations thereof.

Disclosed herein, are methods of detecting a target nucleic acid in a sample. In an aspect, a method of detecting a target nucleic acid in a sample comprises: a. contacting the sample with: i. an effector protein, wherein the effector protein comprises an amino acid sequence that is at least 70%, at least 75%, at least 80%, at least 85%, at least 90%, at least 95%, or 100% identical to any one of SEQ ID NOs: 1-4, ii. an engineered guide nucleic acid, and iii. a detection reagent that is cleaved in the presence of the effector protein, the engineered guide nucleic acid, and the target nucleic acid; and b. detecting a signal produced by cleavage of the detection reagent, thereby detecting the target nucleic acid in the sample.

In some embodiments, the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21.

Disclosed herein, are methods of detecting a target nucleic acid in a sample. In an aspect, a method of detecting a target nucleic acid in a sample comprises: a. contacting the sample with: i. an effector protein, wherein the effector protein comprises an amino acid sequence selected from any one of SEQ ID NOS: 1-4, ii. an engineered guide nucleic acid, and iii a detection reagent that is cleaved in the presence of the effector protein, the engineered guide nucleic acid, and a target nucleic acid, wherein the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T; and b. detecting a signal produced by cleavage of the detection reagent, thereby detecting the target nucleic acid in the sample.

In some embodiments, the method comprises amplifying the target nucleic acid. In some embodiments, amplifying is performed before contacting. In some embodiments, amplifying is performed during contacting. In some embodiments, detecting is performed at a temperature of at least about 40° C. In some embodiments, detecting is performed at the temperature of at least about 45° C. In some embodiments, detecting is performed at the temperature of at least about 50° C. In some embodiments, detecting is performed at the temperature of at least about 55° C. In some embodiments, detecting is performed at the temperature of at least about 60° C. In some embodiments, detecting is performed at the temperature of at least about 65° C. In some embodiments, detecting is performed at about 40° C. In some embodiments, detecting is performed at about 45° C. In some embodiments, detecting is performed at about 50° C. In some embodiments, detecting is performed at about 55° C. In some embodiments, detecting is performed at about 60° C. In some embodiments, detecting is performed at about 65° C. In some embodiments, detecting is performed at a range from about 40° C. to about 75° C. In some embodiments, detecting is performed at a range from about 55° C. to about 65° C. In some embodiments, amplifying is performed at a temperature of at least about 40° C. In some embodiments, amplifying is performed at the temperature of at least about 45° C. In some embodiments, amplifying is performed at the temperature of at least about 50° C. In some embodiments, amplifying is performed at the temperature of at least about 55° C. In some embodiments, amplifying is performed at the temperature of at least about 60° C. In some embodiments, amplifying is performed at the temperature of at least about 65° C. In some embodiments, amplifying is performed at about 40° C. In some embodiments, amplifying is performed at about 45° C. In some embodiments, amplifying is performed at about 50° C. In some embodiments, amplifying is performed at about 55° C. In some embodiments, amplifying is performed at about 60° C. In some embodiments, amplifying is performed at about 65° C. In some embodiments, amplifying is performed at a range from about 40° C. to about 75° C. In some embodiments, amplifying is performed at a range from about 55° C. to about 65° C.

In some embodiments, the engineered guide nucleic acid comprises a crRNA, a tracrRNA, or a combination thereof. In some embodiments, the engineered guide nucleic acid is a single guide RNA (sgRNA). In some embodiments, the effector protein provides cis-cleavage activity on the target nucleic acid. In some embodiments, the effector protein provides transcollateral cleavage activity on the target nucleic acid. In some embodiments, the transcollateral cleavage activity cleaves a single strand of the target nucleic acid. In some embodiments, the transcollateral cleavage activity cleaves the single strand of the target nucleic acid in a sequence non-specific manner.

In some embodiments, the effector protein, the engineered guide nucleic acid, and reporter nucleic acid are formulated in a solution, wherein the solution comprises at least one of a buffering agent, a salt, a crowding agent, a detergent, a reducing agent, a competitor, and a detection agent. In some embodiments, the pH of the solution is selected from at least about 6.0, at least about 6.5, at least about 7.0, at least about 7.5, at least about 8.0, at least about 8.5, or at least about 9. In some embodiments, the pH of the solution ranges from at least about 6.0, at least about 6.5, at least about 7.0, at least about 7.5, at least about 8.0, at least about 8.5, or at least about 9 to about 10, about 11, or about 12. In some embodiments, the pH of the solution is from about 6 to about 12. In some embodiments, the salt is selected from the group consisting a magnesium salt, a potassium salt, a sodium salt, and a calcium salt. In some embodiments, the concentration of the salt in the solution is selected from at least about 1 millimolar (mM), at least about 3 mM, at least about 5 mM, at least about 7 mM, at least about 9 mM, at least about 11 mM, at least about 13 mM, or at least about 15 mM. In some embodiments, the concentration of the salt in the solution is from about 3 mM to about 200 mM. In some embodiments, the concentration of the salt in the solution is from about 15 mM to about 20 mM. In some embodiments, the concentration of the salt in the solution is from about 5 mM to about 10 mM. In some embodiments, the concentration of the salt in the solution is below about 200 mM, wherein the salt is a monovalent salt (e.g., NaCl, KCl, or KOAc). In some embodiments, the concentration of the salt in the solution is from about 15 mM to about 20 mM, wherein the salt is a divalent salt (e.g., Mg²⁺).

In some embodiments, the detection reagent is selected from a reporter nucleic acid, a detection moiety, an additional effector protein, or a combination thereof. In some embodiments, the detection reagent is the reporter nucleic acid. In some embodiments, the reporter nucleic acid comprises a fluorophore, a quencher, or a combination thereof. In some embodiments, the reporter nucleic acid comprises the fluorophore. In some embodiments, the reporter nucleic acid comprises the quencher. In some embodiments, the reporter nucleic acid is in the form of single stranded deoxyribonucleic acid (DNA).

In some embodiments, the method further comprises reverse transcribing the target nucleic acid, amplifying the target nucleic acid, in vitro transcribing the target nucleic acid, or any combination thereof. In some embodiments, the method comprises reverse transcribing the target nucleic acid and/or amplifying the target nucleic acid before contacting the sample with the effector protein, the engineered guide nucleic acid, and the detection reagent. In some embodiments, the method comprises comprising reverse transcribing the target nucleic acid and/or amplifying the target nucleic acid after contacting the sample with the effector protein, the engineered guide nucleic acid, and the detection reagent.

In some embodiments, the contacting and the reverse transcribing are carried out at a same temperature. In some embodiments, the detecting and the reverse transcribing are carried out at a same temperature. In some embodiments, the contacting, the detecting, and the reverse transcribing are carried out at the same temperature. In some embodiments, the contacting and the amplifying are carried out at the same temperature. In some embodiments, the detecting and the amplifying are carried out at the same temperature. In some embodiments, the contacting, the detecting, and the amplifying are carried out at the same temperature. In some embodiments, the contacting, the detecting, the reverse transcribing, and the amplifying are carried out at the same temperature.

In some embodiments, the contacting and the reverse transcribing are carried out in a single reaction chamber. In some embodiments, the detecting and the reverse transcribing are carried out in a single reaction chamber. In some embodiments, the contacting, the detecting, and the reverse transcribing are carried out in a single reaction chamber. In some embodiments, the contacting and the amplifying are carried out in a single reaction chamber. In some embodiments, the detecting and the amplifying are carried out in a single reaction chamber. In some embodiments, the contacting, the detecting, and the amplifying are carried out at the single reaction chamber. In some embodiments, the contacting, the detecting, the reverse transcribing, and the amplifying are carried out at the single reaction chamber.

In some embodiments, amplifying the target nucleic acid comprises at least one amplification reagent. In some embodiments, the at least one amplification reagent is selected from the group consisting of a primer, an activator, a dNTP, an rNTP, and combinations thereof. In some embodiments, amplifying the target nucleic acid comprises isothermal amplification.

In some embodiments, the target nucleic acid is from a pathogen. In some embodiments, the pathogen is a virus or a bacterium. In some embodiments, the pathogen is a virus. In some embodiments, the virus is a SARS-CoV-2 virus or a variant thereof, an influenza A virus, an influenza B virus, a human papillomavirus, a herpes simplex virus, or a combination thereof. In some embodiments, the pathogen is a bacterium. In some embodiments, the bacterium is a Chlamydia trachomatis. In some embodiments, the target nucleic acid is a ribonucleic acid (RNA). In some embodiments, the target nucleic acid is a deoxyribonucleic acid (DNA).

In some embodiments, the target nucleic acid is from a sample. In some embodiments, the sample comprises blood, serum, plasma, saliva, urine, a mucosal sample, a peritoneal sample, cerebrospinal fluid, gastric secretions, nasal secretions, sputum, pharyngeal exudates, urethral or vaginal secretions, an exudate, an effusion, a tissue sample, or any combination thereof.

Disclosed herein, are methods of modifying a target nucleic acid. In an aspect, a method of modifying a target nucleic acid method comprises contacting an effector protein and an engineered guide nucleic acid with a target nucleic acid, wherein the effector protein comprises an amino acid sequence that is at least 70%, at least 75%, at least 80%, at least 85%, at least 90%, at least 95%, or 100% identical to any one of SEQ ID NOs: 1-4, thereby modifying the target nucleic acid.

In some embodiments, the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21. In some embodiments, the target nucleic acid comprises a protospacer adjacent motif (PAM) sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T, thereby modifying the target nucleic acid.

In some embodiments, modifying the target nucleic acid comprises cleaving the target nucleic acid, deleting a nucleotide of the target nucleic acid, inserting a nucleotide into the target nucleic acid, substituting a nucleotide of the target nucleic acid with a donor nucleotide or an additional nucleotide, or any combination thereof. In some embodiments, modifying the target nucleic acid comprises substituting the nucleotide of the target nucleic acid with the donor nucleotide or the additional nucleotide. In some embodiments, the method comprises contacting the target nucleic acid with the donor nucleic acid.

In some embodiments, the target nucleic acid comprises a mutation associated with a disease. In some embodiments, the mutation is suspected to cause the disease. In some embodiments, the disease comprises, at least in part, a cancer, an inherited disorder, an ophthalmological disorder, a neurological disorder, a blood disorder, a metabolic disorder, or a combination thereof. In some embodiments, the disease comprises, at least in part, a neurological disorder. In some embodiments, the neurological disorder comprises Duchenne muscular dystrophy, myotonic dystrophy Type 1, or cystic fibrosis. In some embodiments, the neurological disorder is a neurodegenerative disease.

In some embodiments, the target nucleic acid is encoded by a gene selected from TABLE 4. In some embodiments, the target nucleic acid is PCSK9, or a portion thereof. In some embodiments, the target nucleic acid is TRAC, B2M, PD1, or a portion thereof. In some embodiments, contacting the effector protein and the engineered guide nucleic acid with the target nucleic acid occurs in vitro, in vivo, or ex vivo. In some embodiments, contacting the effector protein and the engineered guide nucleic acid with the target nucleic acid occurs in vitro. In some embodiments, contacting the effector protein and the engineered guide nucleic acid with the target nucleic acid occurs in vivo. In some embodiments, contacting the effector protein and the engineered guide nucleic acid with the target nucleic acid occurs ex vivo.

Disclosed herein, are methods of generating a recombinant cell. In an aspect, a method of generating a recombinant cell the method comprises providing an effector protein and an engineered guide nucleic acid to a target cell, wherein the effector protein comprises an amino acid sequence that is at least 70%, at least 75%, at least 80%, at least 85%, at least 90%, at least 95%, or 100% identical to any one of SEQ ID NOs: 1-4, thereby generating the recombinant cell from the target cell.

In some embodiments, the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21. In some embodiments, the target nucleic acid comprises a protospacer adjacent motif (PAM) sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T, thereby generating the recombinant cell from the target cell.

In some embodiments, the method comprises providing a nucleic acid encoding the effector protein. In some embodiments, the nucleic acid encodes the engineered guide nucleic acid. In some embodiments, providing the effector protein and the engineered guide nucleic acid to the target cell comprises electroporation, acoustic poration, optoporation, viral vector-based delivery, induced transduction by osmocytosis and propanebetaine (iTOP), nanoparticle delivery, cell-penetrating peptide (CPP) delivery, DNA nanostructure delivery, or any combination thereof. In some embodiments, the nanoparticle delivery comprises lipid nanoparticle delivery or gold nanoparticle delivery.

In some embodiments, providing the effector protein and the engineered guide nucleic acid to the target cell generates a double-stranded break in the genome of the target cell, and optionally, the method comprises detecting the double-stranded break. In some embodiments, the method comprises repairing the double-stranded break, and wherein the repairing results in an insertion-deletion (indel) in the genome of the target cell. In some embodiments, the method further comprises delivering a donor nucleic acid to the target cell. In some embodiments, the donor nucleic acid is incorporated into the genome of the target cell, and optionally wherein the method comprises detecting the incorporation of the donor nucleic acid in the genome of the target cell.

In some embodiments, the target cell is a eukaryotic cell. In some embodiments, the target cell is a mammalian cell. In some embodiments, the target cell is a cancer cell, an animal cell, an HEK293 cell, or an immune cell. In some embodiments, the target cell is a Chinese hamster ovary cell. In some embodiments, the target cell is a prokaryotic cell. In some embodiments, a recombinant cell is generated by any method described thereof. In some embodiments, the recombinant cell is a T cell. In some embodiments, the T cell is a natural killer T (NKT) cell. In some embodiments, the recombinant cell is an induced pluripotent stem cell (iPS). In some embodiments, a population of recombinant cells is generated by any method described thereof. In some embodiments, the population of recombinant cells comprises a T cell. In some embodiments, the T cell is an NKT cell. In some embodiments, the population of recombinant cells comprises an iPS. In some embodiments, a progeny cell comprises the progeny cell of any recombinant cell described thereof. In some embodiments, the progeny cell is a T cell. In some embodiments, the T cell is a natural killer T (NKT) cell. In some embodiments, the progeny cell is an induced pluripotent stem cell (iPS).

### BRIEF DESCRIPTION OF THE DRAWINGS

FIG. 1 shows performance of CasM.21526 (SEQ ID NO: 2) in various buffers.

FIG. 2 shows performance of CasM.21526 (SEQ ID NO: 2) with various additives.

FIG. 3 shows performance of CasM.21526 (SEQ ID NO: 2) at 40° C. to 65° C. at various concentrations of target dsDNA.

FIG. 4 shows performance of CasM.21526 (SEQ ID NO: 2) at 40° C. to 65° C. at various concentrations of target dsDNA.

FIG. 5 shows performance of CasM.21526 (SEQ ID NO: 2) at 80° C. to 90° C. at various concentrations of target dsDNA.

FIG. 6 shows performance of CasM.21526 (SEQ ID NO: 2) after thermocycling between various temperatures.

FIG. 7 shows threshold of detection results for CasM.21526 (SEQ ID NO: 2) and Cas12 Variant (SEQ ID NO: 40).

FIG. 8 shows catalytic efficiency results of CasM.21526 (SEQ ID NO: 2), Cas12 Variant (SEQ ID NO: 40), and CasM26 (SEQ ID NO: 48).

FIG. 9 shows performance of CasM.21526 (SEQ ID NO: 2) for SNP discrimination at either 70° C. or 37° C.

FIG. 10 shows performance of CasM.21526 (SEQ ID NO: 2) in a one-pot DETECTR reaction at 62° C. “NTC”=no template control.

FIG. 11 shows performance of CasM.21526 (SEQ ID NO: 2) and Cas14a.1 (SEQ ID NO: 41) in a one-pot DETECTR reaction at 55° C. “NTC”=no template control.

FIG. 12 shows lateral flow assay results after a DETECTR reaction using CasM.21526 (SEQ ID NO: 2) and hydrogel-immobilized reporters.

FIG. 13 shows performance of CasM.21526 (SEQ ID NO: 2) in a digital droplet DETECTR reaction at 60° C.

### DETAILED DESCRIPTION

It is to be understood that both the foregoing general description and the following detailed description are exemplary, and explanatory only and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “about” in reference to a number or range of numbers is understood to mean the stated number and numbers +/−10% thereof, or 10% below the lower listed limit and 10% above the higher listed limit for the values listed for a range.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof.

As used herein, the terms “percent identity,” “% identity,” and “% identical” refer to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs may be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95). For the purposes of calculating identity to the sequence, extensions, such as tags, are not included.

As used herein, a “one-pot” reaction refers to a reaction in which more than one reaction occurs in a single volume alongside a programmable nuclease-based detection (e.g., DETECTR) assay. For example, in a one-pot assay, sample preparation, reverse transcription, amplification, in vitro transcription, or any combination thereof, and programmable nuclease-based detection (e.g., DETECTR) assays are carried out in a single volume. In some embodiments, a) sample preparation, amplification, and detection, b) sample preparation and detection, or c) amplification and detection are carried out within a same volume or region of a device. Readout of the detection (e.g., DETECTR) assay may occur in the single volume or in a second volume. For example, the product of the one-pot DETECTR reaction may be transferred to another volume, applied to a lateral flow strip, etc. for signal generation and indirect detection of reporter cleavage by a sensor or detector (or by eye in the case of a colorimetric signal).

The terms, “amplification” and “amplifying,” as used herein, refer to a process by which a nucleic acid molecule is enzymatically copied to generate a plurality of nucleic acid molecules containing the same sequence as the original nucleic acid molecule or a distinguishable portion thereof.

The term, “base editing enzyme,” as used herein refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term, “base editor,” as used herein refers to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term, “catalytically inactive effector protein,” as used herein refers to an effector protein that is modified relative to a naturally-occurring effector protein to have a reduced or eliminated catalytic activity relative to that of the naturally-occurring effector protein but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

The term, “cis cleavage,” as used herein refers to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The term, “complementary,” as used herein with reference to a nucleic acid refers to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term, “cleavage assay,” as used herein refers to an assay designed to visualize, quantitate or identify cleavage of a nucleic acid. In some cases, the cleavage activity may be cis-cleavage activity. In some cases, the cleavage activity may be trans-cleavage activity.

The term, “clustered regularly interspaced short palindromic repeats (CRISPR),” as used herein refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The term, “CRISPR RNA (crRNA),” as used herein refers to a nucleic acid comprising a first sequence, often referred to as a “spacer sequence,” that hybridizes to a target sequence of a target nucleic acid, and a second sequence that either a) hybridizes to a portion of a tracrRNA or b) is capable of being non-covalently bound by an effector protein. In some embodiments, the crRNA is covalently linked to an additional nucleic acid (e.g., a tracrRNA), wherein the additional nucleic acid interacts with the effector protein.

The term, “detectable signal,” as used herein refers to a signal that can be detected using optical, fluorescent, chemiluminescent, electrochemical and other detection methods known in the art.

The term, “detecting a nucleic acid” and its grammatical equivalents, as used herein refers to detecting the presence or absence of the target nucleic acid in a sample that potentially contains the nucleic acid being detected.

The term, “donor nucleic acid,” as used herein refers to nucleic acid that is incorporated into a target nucleic acid.

The term, “donor nucleotide,” as used herein refers to a single nucleotide that is incorporated into a target nucleic acid. A nucleotide is typically inserted at a site of cleavage by an effector protein.

The term, “effector protein,” as used herein refers to a protein, polypeptide, or peptide that non-covalently binds to a guide nucleic acid to form a complex that contacts a target nucleic acid, wherein at least a portion of the guide nucleic acid hybridizes to a target sequence of the target nucleic acid. In some embodiments, the complex comprises multiple effector proteins. In some embodiments, the effector protein modifies the target nucleic acid when the complex contacts the target nucleic acid. In some embodiments, the effector protein does not modify the target nucleic acid, but it is fused to a fusion partner protein that modifies the target nucleic acid. A non-limiting example of modifying a target nucleic acid is cleaving (hydrolysis) of a phosphodiester bond. Additional examples of modifying target nucleic acids are described herein and throughout. In some embodiments, the term, “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). The effector protein may be a Cas protein (i.e., an effector protein of a CRISPR-Cas system).

The term, “functional fragment,” as used herein refers to a fragment of a protein that retains some function relative to the entire protein. Non-limiting examples of functions are nucleic acid binding, protein binding, nuclease activity, nickase activity, deaminase activity, demethylase activity, or acetylation activity.

The terms, “fusion effector protein,” “fusion protein,” and “fusion polypeptide,” as used herein refer to a protein comprising at least two heterologous polypeptides. Often a fusion effector protein comprises an effector protein and a fusion partner protein. In general, the fusion partner protein is not an effector protein. Examples of fusion partner proteins are provided herein.

The terms, “fusion partner protein” or “fusion partner,” as used herein refer to a protein, polypeptide or peptide that is fused to an effector protein. The fusion partner generally imparts some function to the fusion protein that is not provided by the effector protein. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate or increase expression of a target nucleic acid via additional proteins or nucleic acid modifications to the target sequence.

The term, “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some instances, the heterologous protein exhibits an activity (e.g., enzymatic activity) when it is fused to the effector protein. In some instances, the heterologous protein exhibits increased or reduced activity (e.g., enzymatic activity) when it is fused to the effector protein, relative to when it is not fused to the effector protein. In some instances, the heterologous protein exhibits an activity (e.g., enzymatic activity) that it does not exhibit when it is fused to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The term, “in vitro,” as used herein is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed. The term “in vivo” is used to describe an event that takes place in a subject's body. The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term, “functional domain,” as used herein refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “guide nucleic acid,” as used herein refers to a nucleic acid comprising: a first nucleotide sequence that hybridizes to a target nucleic acid; and a second nucleotide sequence that capable of being non-covalently bound by an effector protein. The first sequence may be referred to herein as a spacer sequence. The second sequence may be referred to herein as a repeat sequence. In some embodiments, the first sequence is located 5′ of the second nucleotide sequence. In some embodiments, the first sequence is located 3′ of the second nucleotide sequence.

The term, “linked amino acids,” as used herein refers to at least two amino acids linked by an amide bond.

The term, “linker,” as used herein refers to a bond or molecule that links a first polypeptide or polynucleotide to a second polypeptide or polynucleotide. A “peptide linker” comprises at least two amino acids linked by an amide bond.

The term, “mutation associated with a disease,” as used herein refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms, “non-naturally occurring” and “engineered,” as used herein are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term, “nuclear localization signal,” as used herein refers to an entity (e.g., peptide) that facilitates localization of a nucleic acid, protein, or small molecule to the nucleus, when present in a cell that contains a nuclear compartment.

The term, “nuclease activity,” as used herein refers to the enzymatic activity of an enzyme which allows the enzyme to cleave the phosphodiester bonds between the nucleotide subunits of nucleic acids; the term “endonuclease activity” refers to the enzymatic activity of an enzyme which allows the enzyme to cleave the phosphodiester bond within a polynucleotide chain. An enzyme with nuclease activity may be referred to as a “nuclease.”

The term, “protospacer adjacent motif (PAM),” as used herein refers to a nucleotide sequence found in a target nucleic acid that directs an effector protein to modify the target nucleic acid at a specific location. A PAM sequence may be required for a complex having an effector protein and a guide nucleic acid to hybridize to and modify the target nucleic acid. However, a given effector protein may not require a PAM sequence being present in a target nucleic acid for the effector protein to modify the target nucleic acid.

The term, “recombinant,” as used herein as applied to proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms.

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. The term, “recombinant polypeptide,” refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

The terms, “reporter” and “reporter nucleic acid,” are used interchangeably herein to refer to a non-target nucleic acid molecule that can provide a detectable signal upon cleavage by an effector protein. Examples of detectable signals and detectable moieties that generate detectable signals are provided herein.

The term, “sample,” as used herein generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term, “subject,” as used herein can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a member of the animal kingdom. The subject can be a mammal. The mammal can be a human. The subject may be diagnosed or suspected of being at high risk for a disease. In some instances, the subject is not necessarily diagnosed or suspected of being at high risk for the disease. As used herein, the terms “individual,” “subject,” and “patient” are used interchangeably.

The term, “target nucleic acid,” as used herein refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein when used in reference to a target nucleic acid refers to a sequence of nucleotides that hybridizes to an equal length portion of a guide nucleic acid. Hybridization of the guide nucleic acid to the target sequence may bring an effector protein into contact with the target nucleic acid.

As used herein, the term, “thermostability,” refers to the stability of a composition disclosed herein at one or more temperatures. Stability may be assessed by the ability of the composition to perform an activity, e.g., cleaving, nicking, or modifying a target nucleic acid. Improving thermostability means improving the quantity or quality of the activity at one or more temperatures.

The term, “trans cleavage,” is used herein in reference to cleavage (hydrolysis of a phosphodiester bond) of one or more nucleic acids by an effector protein that is complexed with a guide nucleic acid and a target nucleic acid. The one or more nucleic acids may include the target nucleic acid as well as non-target nucleic acids.

The term, “trans-activating RNA (tracrRNA),” as used herein refers to a nucleic acid that comprises a first sequence that is capable of being non-covalently bound by an effector protein. TracrRNAs may comprise a second sequence that hybridizes to a portion of a crRNA, which may be referred to as a repeat hybridization sequence. In some embodiments, tracrRNAs are covalently linked to a crRNA.

The term, “transcriptional activator,” as used herein refers to a polypeptide or a fragment thereof that can activate or increase transcription of a target nucleic acid molecule.

The term, “transcriptional repressor,” as used herein refers to a polypeptide or a fragment thereof that is capable of arresting, preventing, or reducing transcription of a target nucleic acid.

The terms, “treatment” or “treating,” as used herein are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “viral vector,” as used herein refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viral vectors include retroviral vectors (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector may be replication competent, replication deficient or replication defective.

A “genetic disease”, as used herein, refers to a disease, disorder, condition, or syndrome caused by one or more mutations in the DNA of an organism. Mutations can be due to several different cellular mechanisms, including, but not limited to, an error in DNA replication, recombination, or repair, or due to environmental factors. A genetic disease comprises, in some embodiments, a single gene disorder, a chromosome disorder, or a multifactorial disorder.

A “syndrome”, as used herein, refers to a group of symptoms which, taken together, characterize a condition.

In some instances, a DNA sequence disclosed herein is interchangeable with a similar RNA sequence. In some cases, an RNA sequence disclosed herein is interchangeable with a similar DNA sequence. In some cases, nucleic acids of the disclosure refer to an RNA sequence. In some cases, nucleic acids of the disclosure refer to a DNA sequence. In some instances, uracils and thymines of a nucleic acid may be interchanged in a sequence provided herein. In some instances, ribose sugars and deoxyribose sugars of a nucleic acid may be interchanged in a sequence provided herein.

Disclosed herein are non-naturally occurring compositions and systems comprising at least one of an effector protein and an engineered guide nucleic acid. In some embodiments, an effector protein and an engineered guide nucleic acid refer to an effector protein and an engineered guide nucleic acid, respectively, that are not found in nature. In some instances, systems and compositions herein comprise at least one non-naturally occurring component. For example, compositions and systems may comprise an engineered guide nucleic acid, wherein the sequence of the engineered guide nucleic acid is different or modified from that of a naturally-occurring guide nucleic acid. In some instances, compositions and systems comprise at least two components that do not naturally occur together. For example, compositions and systems may comprise an engineered guide nucleic acid comprising a repeat region and a spacer region which do not naturally occur together. Also, by way of example, compositions and systems may comprise an engineered guide nucleic acid and an effector protein that do not naturally occur together.

In some instances, the engineered guide nucleic acid comprises a non-natural nucleobase sequence. The non-natural sequence may comprise a portion of a naturally-occurring sequence, wherein the portion of the naturally-occurring sequence is not present in nature absent the remainder of the naturally-occurring sequence. In some instances, the engineered guide nucleic acid comprises two naturally-occurring sequences arranged in an order or proximity that is not observed in nature. In some instances, compositions and systems comprise a ribonucleotide complex comprising an effector protein and an engineered guide nucleic acid that do not occur together in nature. Engineered guide nucleic acids may comprise a first sequence and a second sequence that do not occur naturally together. For example, an engineered guide nucleic acid may comprise a sequence of a naturally-occurring repeat region and a spacer region that is complementary to a naturally-occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The engineered guide nucleic acid may comprise a third sequence disposed at a 3′ or 5′ end of the engineered guide nucleic acid, or between the first and second sequences of the engineered guide nucleic acid. For example, an engineered guide nucleic acid may comprise a naturally occurring crRNA and tracrRNA coupled by a linker sequence.

In some instances, compositions and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

In some instances, compositions and systems provided herein comprise a multi-vector system encoding an effector protein and an engineered guide nucleic acid described herein, wherein the engineered guide nucleic acid and the effector protein are encoded by different vectors. In some embodiments, the engineered guide and the effector protein are encoded by different vectors of the system.

### I. Effector Proteins

Disclosed herein are effector proteins (e.g., programmable nucleases) and uses thereof, e.g., detection and editing of target nucleic acids. In some instances, effector proteins provided herein comprise nucleic acid cleavage activity. In some instances, effector proteins cleave or nick single-stranded nucleic acids, double, stranded nucleic acids, or a combination thereof. In some cases, effector proteins cleave single-stranded nucleic acids. In some cases, effector proteins cleave double-stranded nucleic acids. In some cases, effector proteins nick double-stranded nucleic acids. In many cases, the guide RNAs of effector proteins hybridize to ssDNA or dsDNA. However, the trans cleavage activity of the effector proteins disclosed herein is typically directed towards ssDNA.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein. An effector protein may modify a nucleic acid by cis cleavage or trans cleavage. The modification of the target nucleic acid generated by an effector protein may, as a non-limiting example, result in modulation of the expression of the nucleic acid (e.g., increasing or decreasing expression of the nucleic acid) or modulation of the activity of a translation product of the target nucleic acid (e.g., inactivation of a protein binding to an RNA molecule or hybridization). An effector protein may be a CRISPR-associated (“Cas”) protein. An effector protein may function as a single protein, including a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). An effector protein may be a modified effector protein having reduced modification activity (e.g., a catalytically defective effector protein) or no modification activity (e.g., a catalytically inactive effector protein). Accordingly, an effector protein as used herein encompasses a modified or programmable nuclease that does not have nuclease activity.

In some instances, an effector protein comprises an amino acid sequence that is at least 75%, at least 80%, at least 85%, at least 90%, at least 95%, at least 98%, at least 99% or 100% identical to any one of SEQ ID NOs: 1-4, as provided in TABLE 1 below.

TABLE 1
Effector Protein Sequences

| Name | |
|---|---|
| (SEQ ID | |
| NO) | Effector Protein Amino Acid Sequence |
| CasM.21544 | MKNFFQLFTNQYELSKTLRFELRPIENTAELLKKNKIFEKDKKVYEYYQKTKKYFDKLH |
| (SEQ ID | CEFIDEALVNISLPNKDYSIYDKLFFEYKKDKENKKIYKALNEHAKKLRTNIVKDFEKT |
| NO: 1) | AEKWRDEYILSIKDEKSQKKLKQLEGLDLLFKTEVFDFLKKKYPEAQIEGNSIFDSENK |
| | FFTYFDNFHQSRKNFYKDDGTASAIPTRIIDDNLPKFLENKKIFEEEYKVLNLSREEKD |
| | LFRLDFYNKCFTQTGIENYNEIVSKINSKVNKYRQKNKKSKIHFMKELFKQILSKRSKQ |
| | ATEQDTSISIENDSQVFEVLQDFIMETKNCNRMAKNILETFIQHQEEFDLQKVELKGSN |
| | VTRISDMWFTSWFAFGDLLPKNSTGKKLANFISFQDIKNALEKTEIQDVFKKKYAKYNK |
| | ESFYDQFLQIFDYEFHKALNDCEKNIVEAEKMMKVDKVYSNKKGILRNGKNGEKQKEII |
| | KDFADSALSIYQMMKYFALEKGKKPVTDMDEDPRFYNDYKDWCEKSRILLYYNEFRNYL |
| | TKKPYAEDKIKLNFEKSTLLMGWSANEEGNLQYYSSILRKDNKYYLGLMSSASTENINK |
| | KEAFETRDGFYELMNYRQLKSTTIYGSLYKGEYKKEYRKDKETLINQELIERTKKILEK |
| | QKFSYPQLEVILDKKYKAAEDLAKDLGNIVLYNIGFVKISKRYVNNLINENFYLFEISN |
| | KDFLNKKKKGNVNLHTKYFQLLEDKKNLESKKGAILKLSGGGEVFYRKATKNLPKKKDK |
| | KGKEIIDRKRYAEDKILFHLPIVLNIGFKDKQINSKVNQVLATQRKARIIGIDRGEKHL |
| | LYISVIDENANILKIKSLNTIDVPNKKEPDDYHKLLDEKEKERDDERKSWHTIENIKEL |
| | KHGYISQVVNEIDKIIFECLDERILPIIVFEDLNIGEKRGRFKIEKQVYQKFELALAKK |
| | LNYLVSKEKGNYLNAFQFTPPVNNFQDIHKKQVGIIFYIPASFTSAICPVCGERKRLYG |
| | FTFKNINQVKKLLAEHEFNIFYDGKRENFECLASKENEKKENKNGLYTEIFLNKKLFEN |
| | SDIERLENKKSKDNKKWETISFIATEELKKLFEGKIDLKRNIMDQILAGDFRAEFYQQL |
| | IFIINRILNLRNCHAVEHRDFIACPSCGEHSEKNYEKLKSRYAGKEKFEFNGDANGAYN |
| | IARKGILLIKKIRQFAKSNNIEKLMPYDHLQIDMQEWDKFCAKNKS |
| CasM.21526 | MLKSYDYFTKLYSLQKTLRFELKPIGKILEHIKNSGIIESDETLEEQYAIVKNIIDKLH |
| (SEQ ID | RKHIDEALSLVDFTKHLDTLKTFQELYLKRGKTDKEKEELEKLSADLRKLIVSYLKGNV |
| NO: 2) | KEKTQHNLNPIKERFEILFGKELFTNEEFFLLAENEKEKKAIQAFKGFTTYFKGFQENR |
| | KNMYSEEGNSTSIAYRIINENLPLFIENIARFQKVMSTIEKTTIKKLEQNLKTELKKHN |
| | LPGIFTIEYFNNVLIQEGISRYNTIIGGKTTHEGVKIQGLNEIINLYNQQSKDVKLPIL |
| | KPLHKQILSEEYSTSFKIKAFENDNEVLKAIDTFWNEHIEKSIHPVIGNKENILSKIEN |
| | LCDQLQKYKDKDLEKLFIERKNLSTVSHQVYGQWNIIRDALRMHLEMNNKNIKEKDIDK |
| | YLDNDAFSWKEIKDSIKIYKEHVEDAKELNENGIIKYFSAMSINEEDDEKEYSISLIKN |
| | INEKYNNVKSILQEDRTGKSDLHQDKEKVGIIKEFLDSLKQLQWFLRLLYVTVPLDEKD |
| | YEFYNELEVYYEALLPLNSLYNKVRNYMTRKPYSVEKFKLNFNSPILLDGWDKNKETAN |
| | LSIILRKNGKYYLGIMNKENNTIFEYYPGTKSNDYYEKMIYKLLPGPNKMLPKVFFSKK |
| | GLEYYNPPKEILNIYEKGEFKKDKSGNFKKESLHTLIDFYKEAIAKNEDWEVENFKFKN |
| | TKEYEDISQFYRDVEEQGYLITFEKVDANYVDKLVKEGKLYLFQIYNKDESENKKSKGN |
| | PNLHTIYWKGLYDSENLKNVVYKLNGEAEVFYRKKSIDYPEEIYNHGHHKEELLGKENY |
| | PIIKDRRYTQDKFLFHVPITMNFISKEEKRVNQLACEYLSATKEDVHIIGIDRGERHLL |
| | YLSLIDKEGNIKKQLSLNTIKNENYDKEIDYRVKLDEKEKKRDEARKNWDVIENIKELK |
| | EGYMSQVIHIIAKMMVEEKAILIMEDLNIGFKRGRFKVEKQVYQKFEKMLIDKLNYLVE |
| | KNKNPLEPGGSLNAYQLTSKFDSFKKLGKQSGFIFYVPSAYTSKIDPTTGFYNFIQVDV |
| | PNLEKGKEFFSKFEKIIYNTKEDYFEFHCKYGKFVSEPKNKDNDRKTKESLTYYNAIKD |
| | TVWVVCSTNHERYKIVRNKAGYYESHPVDVTKNLKDIFSQANINYNEGKDIKPIIIESN |
| | NAKLLKSIAEQLKLILAMRYNNGKHGDDEKDYILSPVKNKQGKFFCTLDGNQTLPINAD |
| | ANGAYNIALKGLLLIEKIKKQQGKIKDLYISNLEWEMEMMSR |
| CasM.21550 | MQDKTGWSSFTNKYSLSKILRFELKPVGNTQKMLEDDGVFQKDRERQENYKKVKPFMDK |
| (SEQ ID | LHREFIKEALNNLKLEGLTEYFEIFKKFRKDKNNKELKNAEKKLRQIIGRCYTETAQIW |
| NO: 3) | VEKYKEFGFKKKNIGFLFEEGVFELMKLKYGNDEASQIEKNGEVLSIFDGWKGFLGYFK |
| | KFFETRNNFYKDDGTSTAVSTRIINENLKIYLDNLIKYNKIKDKVDFKEADILQENKLN |
| | LSDFFNVESYAKYSLQKGIDYYNEILGGKTLKNGTKLKGLNEVINEYKQKNKSGELSKF |
| | KMLKKQILGEGEDRILFEEIENEDELKDVLKDFFYNADPKITLFKILLEDFFSNTEKYK |
| | DELDKIYENTVAINGILHRWVDDSGVFQKYLFEVLKSNKLVKSNHYDKKEDSYKEPDFI |
| | SFEHIKVALENCERDGLKDKFWKEKYYTKECLTENGLANLWQEFLEIYKCEFKKLYDYK |
| | TDDNDCYLQYRDNYKKYILDANENPKEKSAKDIIKDYLDSVLSIYQLAKYFALEKKKVW |
| | TTDYETGDFYYEYIKFYEDTYEQIIKPYNLVRNYLTRKPINTAKKWKLNEDNAYLASGW |
| | DKDKEVSNLTVILRRDEQYYLAIMKKGKNKIFEKKFSCGEFEKMEYKQIAEASSDIHNL |
| | VLMNDGSCRRCIKMHDKRKYWPLDISIIKEKKSYAKENFVRRDFERFVNYMKKCSLLYW |
| | KEYDLKFSDTSTYKNINDETNEIASQGYKLSESAIPESYINEKNNNGELYLFQIYNKDE |
| | GIKTEGNKNLHTMYWESIFSEENRFRNFIVKLNGKAEIFYRPKSEQVEKEQRNETREII |
| | KNRRYTENKIYFHCPITLNRISRENVKKENNGINNYIATNPNINILGVDRGEKHLVYYA |
| | IVDQDGKLIDAEDATGSENTIGSTDYHRLLEEKAKDREKERKDWDLIRGIKDLKKGYIS |
| | LVVRKIADLAIKYNAIIIFEDLNTRFKQIRGGMEKSVYQQLEKALINKLSELVNKGEKD |
| | PEQAGHLLKAYQLAAPFQTFDKMGRQTGIIFYTQASYTSKIDPITGWRPNLYLKYRNID |
| | DSKESIKKEKSILENKEKNRFEFTYDLKDFVDFEEDKIPEKTEWTLCSSVERHKWNRHM |
| | NNNKGGYEVYKDLTENFYKLFDENNISMNKDIVDQVESISNGNFFRQFIYLENLVCQIR |
| | NTDEKAEDVDKRDFILSPVEPFFDSRRAKDFKAYGDNLPKNGDENGAYNIARKGVLIIK |
| | KIKEYYNQNGSCDKLGWGDLSISHKEWDDFAINN |
| CasM.21530 | MLIKDFTNLYSLNKILRFELVPQGKINDYIDKWIKELELENKHEVLKDKVDETIAKYIE |
| (SEQ ID | NIKDKLNESNYEPETKSKIIQCIENSKSKINYTDLKENSVREVVSIWFKEINNELNEVD |
| NO: 4) | LRKNIKNKINNRIKKLSEIVEDELKNIILEDKNRAKKYEKVKKILDEYHKDFIERVLKN |
| | LDQTDFNKLLKEYLDLYTKKKDKKETKEFVKIKKNLRKKISDILIKNPDYKILDKDKLF |
| | KRGKKSEKKDSSHEENDDTNVGGILEKFLEENSTLKHKLEESLRNNEDETIDLFELLRS |
| | FEGFTTYFRNFHKNRENLYSDEDKFSTIAHRLIHENLPKFIDNIKIYQKAKEKGLPIEE |
| | IQKQLGITESLDDVFSIEYFTKCLTQSGIDRYNYILGGKSVENGQNIRGVNSFINNEIN |
| | QSVSEKKDRVPVMKMLYKLPLFDRISSSFRYDPIENDQDLIERIVSFYERNLTQYIDTT |
| | TSDDTPVNILEMIKELLQNIHNYREGLYVNGGITLIQISQKIFGDWRYIHDVLSYYYDT |
| | IISPLKKDKKGNLKSRLGTEEKQKEKWMKQKQFSVVLIEKALNEYKKVETNEELKNKIT |
| | DTTLCDFFARCGTDKDGKDLFKRIDEKLTEKNSHSESLKNLMNFNFGNERKLMQNESRI |
| | SLIKNFLDAILGDKEDITAGLLHFIKPLIPREEVADKNELFYSEFERYYNLLSEIIPLY |
| | NKVRNYLTQKPFSIEKIKLNFENPMLLAGWDVNKEADNSCVLERKNGFYYLGIMNKYYR |
| | DVFKNYETANDGEDFYEKMVYKLLPGPNKMLPKVFFSEKNIGYENPSDEILRIRNTASY |
| | SKNGKPREGYKKADFSVNDCQQLIDFFKESILKHEDWSKENFQFKPTSRYNDISEFYKD |
| | VKNQGYKINFVKIPSTYINQLVQEGKLYLFKIHNKDFSEKKKPGGKDNLHTLYWKMLES |
| | EENLKDVVEKLDGEAEIFFRPKSIIYDENIWKNGHHNEKLKDKFNYPIIKDRRFALDKI |
| | YFHVPITINFKAGDVPDNEDEEKNNQQNEMKNESFNQKILSFLKDRDDVHIIGIDRGER |
| | HLLYVVVINSDGKIVEQFSLNKIPREKSDVPIDYLDRLNNKEGQLDEARKNWQTIQNIK |
| | ELKEGYLSHVIHIITRLIVKYNAIVVMEDLNSGEKRGRQKVEKQVYQKFEKMLINKLNY |
| | LVFKDKKAGEPGGVLKGLQLTDKFESFKNLGKQSGIIFYVPPAYTSAIDPVTGYVQYLY |
| | PLKRADSIEKAKNFYSKFDSTKYNAEKDRFEFTEDYEKIPNTRYEGKSQWTICTSDEER |
| | YYWDKSLNNGKGGQKEYKTTSEMKQLFKEAAIPYHTGEELKQYIAGISGNPEKKIKDKE |
| | FFNRLNLLLHVILKLRHNNGKDGDKEQDYILSPVEPFFDSRKAKGDLPENADANGAFNI |
| | ARKGLVLLKRLKKMDIDEFEKTKKSKDGKSQWLPHKEWLEFSQEKHAAVLN |

In some cases, an effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to any one of SEQ ID NOs: 1-4. In some instances, an effector protein comprises an amino acid sequence that is at least 75% identical to any one of SEQ ID NOs: 1-4. In some instances, an effector protein comprises an amino acid sequence that is at least 80% identical to any one of SEQ ID NOs: 1-4. In some instances, an effector protein comprises an amino acid sequence that is at least 85% identical to any one of SEQ ID NOs: 1-4. In some instances, an effector protein comprises an amino acid sequence that is at least 90% identical to any one of SEQ ID NOs: 1-4. In some instances, an effector protein comprises an amino acid sequence that is at least 91% identical to any one of SEQ ID NOs: 1-4. In some instances, an effector protein comprises an amino acid sequence that is at least 92% identical to any one of SEQ ID NOs: 1-4. In some instances, an effector protein comprises an amino acid sequence that is at least 93% identical to any one of SEQ ID NOs: 1-4. In some instances, an effector protein comprises an amino acid sequence that is at least 94% identical to any one of SEQ ID NOs: 1-4. In some instances, an effector protein comprises an amino acid sequence that is at least 95% identical to any one of SEQ ID NOs: 1-4. In some instances, an effector protein comprises an amino acid sequence that is at least 96% identical to any one of SEQ ID NOs: 1-4. In some instances, an effector protein comprises an amino acid sequence that is at least 97% identical to any one of SEQ ID NOs: 1-4. In some instances, an effector protein comprises an amino acid sequence that is at least 98% identical to any one of SEQ ID NOs: 1-4. In some instances, an effector protein comprises an amino acid sequence that is at least 99% identical to any one of SEQ ID NOs: 1-4. In some instances, an effector protein comprises an amino acid sequence that is identical to any one of SEQ ID NOs: 1-4. In some instances, an effector protein comprises an amino acid sequence that is at least 50%, at least 55%, at least 60%, at least 65%, or at least 70% identical to any one of SEQ ID NOs: 1-4.

In some instances, an effector protein comprises an amino acid sequence that is at least 50%, at least 55%, at least 60%, at least 65%, or at least 70% identical to SEQ ID NO: 1. In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 1. In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 1.

In some instances, an effector protein comprises an amino acid sequence that is at least 50%, at least 55%, at least 60%, at least 65%, or at least 70% identical to SEQ ID NO: 2. In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2. In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2.

In some instances, an effector protein comprises an amino acid sequence that is at least 50%, at least 55%, at least 60%, at least 65%, or at least 70% identical to SEQ ID NO: 3. In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 3. In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 3.

In some instances, an effector protein comprises an amino acid sequence that is at least 50%, at least 55%, at least 60%, at least 65%, or at least 70% identical to SEQ ID NO: 4. In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 4. In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 4.

In some instances, an effector protein does not comprise the amino acid sequence MEEK (SEQ ID NO: 47) at the N terminus.

In some instances, an effector protein does not comprise the amino acid sequence MEEK (SEQ ID NO: 47).

In some instances, an effector protein comprises an amino acid sequence that is at least 50%, at least 55%, at least 60%, at least 65%, or at least 70% identical to SEQ ID NO: 2 and does not comprise the amino acid sequence MEEK (SEQ ID NO: 47) at the N terminus. In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2 and does not comprise the amino acid sequence MEEK (SEQ ID NO: 47) at the N terminus. In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2 and does not comprise the amino acid sequence MEEK (SEQ ID NO: 47) at the N terminus.

In some instances, an effector protein comprises an amino acid sequence that is at least 50%, at least 55%, at least 60%, at least 65%, or at least 70% identical to SEQ ID NO: 2 and does not comprise the amino acid sequence MEEK (SEQ ID NO: 47). In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2 and does not comprise the amino acid sequence MEEK (SEQ ID NO: 47). In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2 and does not comprise the amino acid sequence MEEK (SEQ ID NO: 47).

In some instances, an effector protein comprises an amino acid sequence that is at least 50%, at least 55%, at least 60%, at least 65%, or at least 70% identical to SEQ ID NO: 2 and does not comprise the amino acid sequence MEEK (SEQ ID NO: 47) upstream of the amino acid residue corresponding to amino acid residue at the first position of SEQ ID NO: 2. In some instances, the effector protein comprises an amino acid sequence that is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2 and does not comprise the amino acid sequence MEEK (SEQ ID NO: 47) upstream of the amino acid residue corresponding to amino acid residue at the first position of SEQ ID NO: 2. In some cases, the amino acid sequence of the effector protein is at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO: 2 and does not comprise the amino acid sequence MEEK (SEQ ID NO: 47) upstream of the amino acid residue corresponding to amino acid residue at the first position of SEQ ID NO: 2.

In some instances, an effector protein may function as an endonuclease that catalyzes cleavage at a specific position within a target nucleic acid. In some instances, an effector protein is capable of catalyzing non-sequence-specific cleavage of a single stranded nucleic acid. In some cases, an effector protein is activated to perform trans cleavage activity after binding of an engineered guide nucleic acid with a target nucleic acid. This trans cleavage activity is also referred to as “collateral” or “transcollateral” cleavage. Trans cleavage may occur near, but not within or directly adjacent to, the region of the target nucleic acid that is hybridized to the guide nucleic acid. Trans cleavage activity may be triggered by the hybridization of the guide nucleic acid to the target nucleic acid. Trans cleavage activity may be non-specific cleavage of nearby single-stranded nucleic acid by the activated effector, such as trans cleavage of detector nucleic acids with a detection moiety. In some instances, the trans cleavage activity of an effector protein is measured by a trans cleavage assay. An example trans cleavage assay may be a DETECTR assay. An example trans cleavage assay may be described in Examples 2-4. An example trans cleavage assay may also be described elsewhere in this disclosure.

In some cases, the effector protein comprises a catalytically inactive nuclease domain. A catalytically inactive domain of an effector protein may comprise at least 1, at least 2, at least 3, at least 4, or at least 5 mutations relative to a nuclease domain of the effector protein. Said mutations may be present within a cleaving or active site of the nuclease.

In some instances, the effector protein has been modified (also referred to as an engineered protein). In some instances, an engineered protein comprises an amino acid sequence that is at 70%, at least 75%, at least 80%, at least 85%, at least 90%, at least 92%, at least 95%, at least 97%, or at least 99% identical to any one of SEQ ID NOS: 1-4. In some instances, an engineered protein comprises the amino acid sequence of any one of SEQ ID NOS:1-4 fused to a heterologous amino acid sequence or a heterologous molecule. An effector protein disclosed herein or a variant thereof may comprise a nuclear localization signal (NLS). In some cases, the NLS may comprise a sequence of KRPAATKKAGQAKKKKEF (SEQ ID NO: 5). A nucleotide sequence encoding an effector protein may be codon optimized for expression in a specific cell, for example, a bacterial cell, a plant cell, a eukaryotic cell, an animal cell, a mammalian cell, or a human cell. In some embodiments, the nucleotide sequence encoding an effector protein is codon optimized for a human cell.

In some cases, the effector protein is fused or linked to one or more NLS. In some cases, the one or more NLS are fused or linked to the N-terminus of the effector protein. In some cases, the one or more NLS are fused or linked to the C-terminus of the effector protein. In some cases, the NLS are fused or linked to the N-terminus and the C-terminus of the effector protein. In cases where more than one NLS is fused or linked to the effector protein, the more than one NLS can be the same or different.

In some cases, the effector protein comprises an amino acid sequence that is at least 75%, at least 80%, at least 85%, at least 90%, at least 95%, at least 98%, at least 99%, or 100% identical to SEQ ID NO: 2, the N-terminal 25 amino acids includes an NLS and the amino acid sequence LKS.

In some cases, where the effector protein comprises an amino acid sequence that is at least 75%, at least 80%, at least 85%, at least 90%, at least 95%, at least 98%, at least 99%, or 100% identical to SEQ ID NO: 2, the N-terminal 25 amino acids includes an NLS and N-terminal 25 amino acids do not include the amino acid sequence EEK.

In some cases, the effector protein comprises an amino acid sequence that is at least 75%, at least 80%, at least 85%, at least 90%, at least 95%, at least 98%, at least 99%, or 100% identical to SEQ ID NO: 2, the N-terminal 25 amino acids includes a signal peptide and the amino acid sequence LKS.

In some cases, the effector protein comprises an amino acid sequence that is at least 75%, at least 80%, at least 85%, at least 90%, at least 95%, at least 98%, at least 99%, or 100% identical to SEQ ID NO: 2, the N-terminal 25 amino acids includes a signal peptide and N-terminal 25 amino acids do not include the amino acid sequence EEK.

In some cases, the effector protein comprises an amino acid sequence that is at least 75%, at least 80%, at least 85%, at least 90%, at least 95%, at least 98%, at least 99% or 100% identical to any one of SEQ ID NOs: 1-4. In some cases, the effector protein comprises an amino acid sequence that is at least 75%, at least 80%, at least 85%, at least 90%, at least 95%, at least 98%, at least 99% or 100% identical to any one of SEQ ID NO: 1. In some cases, the effector protein comprises an amino acid sequence that is at least 75%, at least 80%, at least 85%, at least 90%, at least 95%, at least 98%, at least 99% or 100% identical to any one of SEQ ID NO: 2. In some cases, the effector protein comprises an amino acid sequence that is at least 75%, at least 80%, at least 85%, at least 90%, at least 95%, at least 98%, at least 99% or 100% identical to any one of SEQ ID NO: 3. In some cases, the effector protein comprises an amino acid sequence that is at least 75%, at least 80%, at least 85%, at least 90%, at least 95%, at least 98%, at least 99% or 100% identical to any one of SEQ ID NO: 4. The effector protein may recognize PAM sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine, wherein N is adenine A, G, C, or T, and wherein Y is a C or T. In some cases, the effector protein may comprise any effector proteins described herein and thereof. In some cases, the compositions may also comprise an engineered guide nucleic acid described herein

In some cases, the effector protein comprises formyl-methionine. In some cases, the effector protein is expressed in bacteria and comprises formyl-methionine at the N-terminus.

In some cases, an effector protein comprises an amino acid sequence that is at least 75%, at least 80%, at least 85%, at least 90%, at least 95%, at least 98%, at least 99%, or 100% identical to SEQ ID NO: 2 comprises formyl-methionine at the N-terminus and the N-terminal amino acids are MLKS (SEQ ID NO: 57).

In some cases, an effector protein comprises an amino acid sequence that is at least 75%, at least 80%, at least 85%, at least 90%, at least 95%, at least 98%, at least 99%, or 100% identical to SEQ ID NO: 2 comprises formyl-methionine at the N-terminus and the N-terminal amino acids are not MEEK (SEQ ID NO: 47).

In some cases, an effector protein comprises an amino acid sequence that is at least 75%, at least 80%, at least 85%, at least 90%, at least 95%, at least 98%, at least 99%, or 100% identical to SEQ ID NO: 2 comprises formyl-methionine at the N-terminus and the effector protein does not include the sequence MEEK (SEQ ID NO: 47).

In some preferred cases, the effector protein does not comprise formyl-methionine.

The invention also provides methods of purifying an effector protein disclosed herein. In some cases, the effector protein is purified following expression in bacteria. In some cases, the effector protein is purified following expression in a eukaryotic cell.

In some cases, compositions comprise an effector protein and a cell. In some embodiments, compositions comprise a cell that expresses an effector protein. In some cases, compositions comprise a nucleic acid encoding an effector protein and a cell. In some embodiments, compositions comprise a cell expressing a nucleic acid encoding an effector protein. In some instances, the cell is a prokaryotic cell. In some instances, the cell is a eukaryotic cell. In some instances, the cell is a mammalian cell.

In some cases, a vector encodes an effector protein comprising an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to any one of SEQ ID NOs: 1-4. In some cases, the effector protein comprises an amino acid sequence that is at least 75%, at least 80%, at least 85%, at least 90%, at least 95%, at least 98%, at least 99%, or 100% identical to SEQ ID NO: 1. In some cases, the effector protein comprises an amino acid sequence that is at least 75%, at least 80%, at least 85%, at least 90%, at least 95%, at least 98%, at least 99%, or 100% identical to SEQ ID NO: 2. In some cases, the effector protein comprises an amino acid sequence that is at least 75%, at least 80%, at least 85%, at least 90%, at least 95%, at least 98%, at least 99%, or 100% identical to SEQ ID NO: 3. In some cases, the effector protein comprises an amino acid sequence that is at least 75%, at least 80%, at least 85%, at least 90%, at least 95%, at least 98%, at least 99%, or 100% identical to SEQ ID NO: 4.

In some instances, an effector protein disclosed herein is encoded by a nucleic acid. In some cases, the nucleic acid is comprised in a vector. In some cases, the nucleic acid encoding the effector protein is DNA or RNA. When the nucleic acid is encoded by RNA, the RNA preferably includes a Shine Dalgarno sequence 5′ from the start codon. Shine Dalgarno sequences are well known to the skilled person. A Shine Dalgarno sequence is typically a purine-rich tract of 3 to 10 nucleotides that centers approximately 10 nucleotides 5′ of the start codon. When the nucleic acid is a DNA, the DNA sequence preferably encodes an RNA that includes a Shine Dalgarno sequence 5′ of the start codon.

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a Shine Dalgarno sequence and the start codon encodes the methionine of the sequence MLKS (SEQ ID NO: 57).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a Shine Dalgarno sequence and the N-terminal amino acids are not MEEK (SEQ ID NO: 47).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a Shine Dalgarno sequence, wherein the center of the Shine Dalgarno sequence is between 6 and 12 nucleotides 5′ of the first nucleotide in the start codon and the start codon encodes the methionine of the sequence MLKS (SEQ ID NO: 57).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a Shine Dalgarno sequence, wherein the center of the Shine Dalgarno sequence is between 6 and 12 nucleotides 5′ of the first nucleotide in the start codon and the start codon does not encode the methionine in the sequence MEEK (SEQ ID NO: 47).

In some cases, the nucleic acid encoding the effector protein includes a TATA box. TATA boxes are cis-regulatory elements in promoters and include repeated T and A base pairs. TATA boxes are well known to the skilled person. A TATA box is typically 20 to 100 base pairs upstream of the transcription start site.

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a TATA box and the start codon encodes the methionine of the sequence MLKS (SEQ ID NO: 57).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a TATA box and the N-terminal amino acids are not MEEK (SEQ ID NO: 47).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a TATA box, wherein the TATA box is between 20 and 100 nucleotides 5′ of the transcription start site and the start codon encodes the methionine of the sequence MLKS (SEQ ID NO: 57).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a TATA box, wherein the TATA box is between 20 and 100 bases 5′ of the transcription start site and the start codon the start codon does not encode the methionine in the sequence MEEK (SEQ ID NO: 47).

In some cases, the nucleic acid encoding the effector protein includes a Kozak sequence. Kozak sequences are well known to the skilled person. For example, when the nucleic acid is RNA, a Kozak sequence may comprise the sequence CRCCAUGG (SEQ ID NO: 46), wherein R is purine and AUG is the start codon.

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a Kozak sequence and the start codon encodes the methionine of the sequence MLKS (SEQ ID NO: 57).

In some cases, a nucleic acid encoding an effector protein comprising an amino acid sequence that is at least 75%, at least 80%, at least 85%, at least 90%, at least 95%, at least 98%, at least 99%, or 100% identical to SEQ ID NO: 2 includes a Kozak sequence and the N-terminal amino acids are not MEEK (SEQ ID NO: 47).

In some preferred cases, the nucleic acid sequence encoding the effector protein is codon optimized.

In some preferred cases, a vector encoding an effector protein disclosed herein comprises a codon optimized nucleic acid sequence encoding the effector protein.

In some embodiments, effector proteins described herein can be isolated and purified for use in compositions, systems, and/or methods described herein. Methods described here can include the step of isolating effector proteins described herein. Compositions and/or systems described herein can further comprise a purification tag that can be attached to an effector protein or a nucleic acid encoding for a purification tag that can be attached to a nucleic acid encoding for an effector protein as described herein. A purification tag, as used herein, can be an amino acid sequence which can attach or bind with high affinity to a separation substrate and assist in isolating the protein of interest from its environment, which can be its biological source, such as a cell lysate. Attachment of the purification tag can be at the N or C terminus of the effector protein. In some instances when a purification tag located at the N terminus of the programmable nuclease, a start codon for the purification tag serves as a start codon for the effector protein as well. Thus, the natural start codon of the effector protein may be removed or absent. Furthermore, an amino acid sequence recognized by a protease or a nucleic acid encoding for an amino acid sequence recognized by a protease, such as TEV protease or the HRV3C protease can be inserted between the purification tag and the effector protein, such that biochemical cleavage of the sequence with the protease after initial purification liberates the purification tag. Purification and/or isolation can be through high performance liquid chromatography (HPLC), exclusion chromatography, gel electrophoresis, affinity chromatography, or other purification technique. Non-limiting examples of purification tags include a histidine tag, e.g., a 6×His tag; a hemagglutinin (HA) tag; a FLAG tag; a Myc tag; and maltose binding protein (MBP). In some embodiments, a programmable nuclease is fused or linked (e.g., via an amide bond) to a fluorescent protein. Non-limiting examples of fluorescent proteins include green fluorescent protein (GFP), yellow fluorescent protein (YFP), red fluorescent protein (RFP), cyan fluorescent protein (CFP), mCherry, and tdTomato.

### A. Engineered Proteins

In some instances, an effector protein disclosed herein is an engineered protein. The engineered protein is not identical to a naturally-occurring protein. The engineered protein may provide enhanced nuclease or nickase activity as compared to a naturally occurring nuclease or nickase. By way of non-limiting example, some engineered proteins exhibit optimal activity at lower salinity and viscosity than the protoplasm of their bacterial cell of origin. Also by way of non-limiting example, bacteria often comprise protoplasmic salt concentrations greater than 250 mM and room temperature intracellular viscosities above 2 centipoise, whereas engineered proteins exhibit optimal activity (e.g., cis-cleavage activity) at salt concentrations below 150 mM and viscosities below 1.5 centipoise. The present disclosure leverages these dependencies by providing engineered proteins in solutions optimized for their activity and stability.

Compositions and systems described herein may comprise an engineered protein in a solution comprising a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise. Compositions and systems may comprise an engineered protein in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. Compositions and systems may comprise an engineered protein and an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

An engineered protein may comprise a modified form of a wildtype counterpart protein. The modified form of the wildtype counterpart may comprise an amino acid change (e.g., deletion, insertion, or substitution) that reduces the nucleic acid-cleaving activity of the effector protein. In some embodiments, the nucleic-acid cleaving activity is target-specific (e.g., cis) cleavage. In some embodiments, the nucleic-acid cleaving activity is transcollateral cleavage. For example, a nuclease domain (e.g., RuvC domain) of a Type V CRISPR/Cas protein may be deleted or mutated so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. Engineered proteins may have no substantial nucleic acid-cleaving activity. Engineered proteins may be enzymatically inactive or “dead,” that is it may bind to a nucleic acid but not cleave it. An enzymatically inactive protein may comprise an enzymatically inactive domain (e.g., inactive nuclease domain). Enzymatically inactive may refer to an activity less than 1%, less than 2%, less than 3%, less than 4%, less than 5%, less than 6%, less than 7%, less than 8%, less than 9%, or less than 10% activity compared to the wild-type counterpart. A dead protein may associate with an engineered guide nucleic acid to activate or repress transcription of a target nucleic acid sequence. In some embodiments, the enzymatically inactive protein is fused with a protein comprising recombinase activity.

### B. Fusion Proteins

In some instances, an effector protein is a fusion protein, wherein the fusion protein comprises a protein comprising the amino acid sequence of any one of SEQ ID NOs: 1-4. In some instances, the fusion protein comprises an effector protein and a fusion partner protein.

A fusion partner protein is also simply referred to herein as a fusion partner. In some cases, the fusion partner promotes the formation of a multimeric complex of the effector protein. In some cases, the fusion partner is an additional effector protein. In some cases, the multimeric complex comprising the effector protein and the additional effector protein binds a guide RNA. The effector proteins of the multimeric complex may bind the guide RNA in an asymmetric fashion. In some cases, one effector protein of the multimeric complex interacts more strongly with the guide RNA than the additional effector protein of the multimeric complex. In some cases, an effector protein interacts more strongly with a target nucleic acid when it is complexed with the guide RNA relative to when the effector protein or the multimeric complex is not complexed with the guide RNA.

In some instances, the fusion partner inhibits the formation of a multimeric complex of the effector protein. In some instances, the fusion partner inhibits the formation of a multimeric complex of the effector protein. By way of non-limiting example, the fusion protein may comprise an effector protein and a fusion partner comprising a Calcineurin A tag, wherein the fusion protein dimerizes in the presence of Tacrolimus (FK506). Also by way of non-limiting example, the fusion protein may comprise an effector protein and a SpyTag configured to dimerize or associate with another effector protein in a multimeric complex.

In some cases, the fusion partner modulates transcription (e.g., inhibits transcription, increases transcription) of a target nucleic acid. In some cases, the fusion partner is a protein (or a domain from a protein) that inhibits transcription, also referred to as a transcriptional repressor. Transcriptional repressors may inhibit transcription via recruitment of transcription inhibitor proteins, modification of target DNA such as methylation, recruitment of a DNA modifier, modulation of histones associated with target DNA, recruitment of a histone modifier such as those that modify acetylation and/or methylation of histones, or a combination thereof. In some cases, the fusion partner is a protein (or a domain from a protein) that increases transcription, also referred to as a transcription activator. Transcriptional activators may promote transcription via recruitment of transcription activator proteins, modification of target DNA such as demethylation, recruitment of a DNA modifier, modulation of histones associated with target DNA, recruitment of a histone modifier such as those that modify acetylation and/or methylation of histones, or a combination thereof. In some cases, the fusion partner is a reverse transcriptase. In some cases, the fusion partner is a base editor. In general, a base editor comprises a deaminase that when fused with an effector protein or a Cas protein changes a nucleobase to a different nucleobase, e.g., cytosine to thymine or guanine to adenine. In some instances, the base editor comprises a deaminase.

In some cases, fusion partners provide enzymatic activity that modifies a target nucleic acid. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity.

In some cases, fusion partners have enzymatic activity that modifies the target nucleic acid. The target nucleic acid may comprise or consist of a ssRNA, a double-stranded RNA (dsRNA), a ssDNA, or a dsDNA. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

In some cases, a fusion partner provides enzymatic activity that modifies a protein (e.g., a histone) associated with a target nucleic acid. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Non-limiting examples of fusion partners that promote or increase transcription include, but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof.

Non-limiting examples of fusion partners that decrease or inhibit transcription include, but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof.

In some cases, the fusion partner has enzymatic activity that modifies a protein associated with a target nucleic acid. The protein may be a histone, an RNA binding protein, or a DNA binding protein. Examples of such protein modification activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some cases, the fusion partner is a chloroplast transit peptide (CTP), also referred to as a plastid transit peptide. In some instances, this targets the fusion protein to a chloroplast. Chromosomal transgenes from bacterial sources must have a sequence encoding a CTP sequence fused to a sequence encoding an expressed protein if the expressed protein is to be compartmentalized in the plant plastid (e.g. chloroplast). The CTP is removed in a processing step during translocation into the plastid. Accordingly, localization of an exogenous protein to a chloroplast is often accomplished by means of operably linking a polynucleotide sequence encoding a CTP sequence to the 5′ region of a polynucleotide encoding the exogenous protein. In some cases, the CTP is located at the N-terminus of the fusion protein. Processing efficiency may, however, be affected by the amino acid sequence of the CTP and nearby sequences at the amino terminus (NH2 terminus) of the peptide.

In some cases, the fusion partner is an endosomal escape peptide. In some cases, an endosomal escape protein comprises the amino acid sequence GLFXALLXLLXSLWXLLLXA (SEQ ID NO: 6), wherein each X is independently selected from lysine, histidine, and arginine. In some cases, an endosomal escape protein comprises the amino acid sequence GLFHALLHLLHSLWHLLLHA (SEQ ID NO: 7). In some cases, the amino acid sequence of the endosomal escape protein is SEQ ID NO: 6 or SEQ ID NO: 7.

In some instances, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some instances, fusion partners that increase or decrease transcription include a transcription activator domain or a transcription repressor domain, respectively.

In some cases, fusion proteins are targeted by an engineered guide nucleic acid (e.g., a guide RNA) to a specific location in the target nucleic acid and exert locus-specific regulation such as blocking RNA polymerase binding to a promoter (which selectively inhibits transcription activator function), and/or modifying the local chromatin status (e.g., when a fusion sequence is used that modifies the target nucleic acid or modifies a protein associated with the target nucleic acid). In some cases, the modifications are transient (e.g., transcription repression or activation). In some cases, the modifications are inheritable. For instance, epigenetic modifications made to a target nucleic acid, or to proteins associated with the target nucleic acid, e.g., nucleosomal histones, in a cell, are observed in cells produced by proliferation of the cell.

Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins. It is understood that a fusion protein may include the entire protein or in some cases may include a fragment of the protein (e.g., a functional domain). In some instances, the functional domain interacts with or binds ssRNA, including intramolecular and/or intermolecular secondary structures thereof, e.g., hairpins, stem-loops, etc.). The functional domain may interact transiently or irreversibly, directly or indirectly. Fusion proteins may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRml60); protein domains responsible for stabilizing RNA (e.g., PABP); proteins and protein domains responsible for repressing translation (e.g., Ago2 and Ago4); proteins and protein domains responsible for stimulating translation (fe.g., Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); proteins and protein domains responsible for RNA localization (e.g., from IMP1, ZBP1, She2p, She3p, and Bicaudal-D); proteins and protein domains responsible for nuclear retention of RNA (e.g., Rrp6); proteins and protein domains responsible for nuclear export of RNA (e.g., TAP, NXF1, THO, TREX, REF, and Aly); proteins and protein domains responsible for repression of RNA splicing (e.g., PTB, Sam68, and hnRNP A1); proteins and protein domains responsible for stimulation of RNA splicing (e.g., Serine/Arginine-rich (SR) domains); proteins and protein domains responsible for reducing the efficiency of transcription (e.g., FUS (TLS)); and proteins and protein domains responsible for stimulating transcription (e.g., CDK7 and HIV Tat). Alternatively, the effector domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627, which is hereby incorporated by reference in its entirety.

In some instances, the fusion partner comprises an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cis-elements that are located in either the core exon region or the exon extension region (i.e., between the two alternative 5′ splice sites). For more examples, see WO2010075303, which is hereby incorporated by reference in its entirety.

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), protein docking elements (e.g., FKBP/FRB, Pill/Aby1, etc.).

In some cases, a terminus of the effector protein is linked to a terminus of the fusion partner through an amide bond. In some cases, an effector protein is coupled to a fusion partner via a linker protein. The linker protein may have any of a variety of amino acid sequences. A linker protein may comprise a region of rigidity (e.g., beta sheet, alpha helix), a region of flexibility, or any combination thereof. In some instances, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. These linkers may be produced by using synthetic, linker-encoding oligonucleotides to couple the proteins, or may be encoded by a nucleic acid sequence encoding a fusion protein (e.g., an effector protein coupled to a fusion partner). Examples of linker proteins include glycine polymers (G)n (SEQ ID NO: 8), glycine-serine polymers (including, for example, (GS)n (SEQ ID NO: 9), GSGGSn (SEQ ID NO: 10), GGSGGSn (SEQ ID NO: 11), and GGGSn (SEQ ID NO: 12), where n is an integer of 1-10), glycine-alanine polymers, and alanine-serine polymers. Exemplary linkers may comprise amino acid sequences including, but not limited to, GGSG (SEQ ID NO: 13), GGSGG (SEQ ID NO: 14), GSGSG (SEQ ID NO: 15), GSGGG (SEQ ID NO: 16), GGGSG (SEQ ID NO: 17), and GSSSG (SEQ ID NO: 18).
II. Guide Nucleic Acids

The compositions, systems, and methods of the present disclosure may comprise an engineered guide nucleic acid or a use thereof. In general, an engineered guide nucleic acid is a nucleic acid molecule that binds to an effector protein, thereby forming a ribonucleoprotein complex (RNP) a target nucleic acid, thereby targeting the RNP to the target nucleic acid. A guide RNA generally comprises a crispr RNA (crRNA), at least a portion of which is complementary to a target sequence of a target nucleic acid. In some instances, the guide RNA comprises a trans-activating crispr RNA (tracrRNA) that interacts with the effector protein. In some cases, the guide RNA is a single guide RNA (sgRNA) (e.g., a crRNA linked to a tracrRNA). In some instances, a crRNA and tracrRNA function as two separate, unlinked molecules. Guide nucleic acids are often referred to as “guide RNA.” However, an engineered guide nucleic acid may comprise deoxyribonucleotides, deoxynucleotides, chemically modified nucleosides, or any combination thereof.

In some cases, engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2—NH—O—CH2—, —CH2—N(CH3)—O—CH2—(known as a methylene (methylimino) or MMI backbone), —CH2—O—N(CH3)—CH2—, —CH2—N(CH3)—N(CH3)—CH2— and —O—N(CH3)—CH2—CH2—(wherein the native phosphodiester internucleotide linkage is represented as —O—P(═O)(OH)—O—CH2—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

The term “guide RNA,” as well as crRNA and tracrRNA, includes guide nucleic acids comprising DNA nucleosides/nucleotides or bases and RNA nucleosides/nucleotides or bases. The guide RNA may be chemically synthesized or recombinantly produced. The sequence of the engineered guide nucleic acid, or a portion thereof, may be different from the sequence of a naturally occurring nucleic acid. The engineered guide nucleic acid may bind an effector protein, wherein the amino acid sequence of the effector protein is at least 70%, at least 75%, at least 80%, at least 85%, at least 90%, at least 95%, at least 97%, at least 98%, or at least 99% identical to any one of SEQ ID NOs: 1-4.

In some cases, the guide RNA is a single guide RNA (sgRNA) comparing a crRNA, and in some instances, a tracrRNA. When used in the context of a sgRNA, the term “tracrRNA” is used for simplicity. However, a tracrRNA sequence linked to a crRNA in a sgRNA may not be functioning in trans and thus may not be considered to be a tracrRNA. For instance, the sgRNA often comprises only a portion of a tracrRNA sequence. In some embodiments, the sgRNA comprises only a portion of a naturally occurring tracrRNA sequence. For example, in some aspects, a sgRNA can include a portion of a tracrRNA that is capable of being non-covalently bound by an effector protein, but does not include all or a part of the portion of a tracrRNA that hybridizes to a portion of a crRNA as found in a dual nucleic acid system. In some aspects, a sgRNA can include a portion of a tracrRNA as well as a portion of a repeat sequence, which can optionally be connected by a linker.

Guide nucleic acids and portions thereof may be found in or identified from a CRISPR array present in the genome of a host organism. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpf1, from Type V systems). A crRNA may also be specifically generated outside of processing of a pre-crRNA and individually contacted to an effector protein in vivo or in vitro.

Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein. Guide nucleic acids may comprise DNA, RNA, or a combination thereof (e.g., RNA with a thymine base). Guide nucleic acids may include a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides. A guide nucleic acid may comprise a CRISPR RNA (crRNA), a short-complementarity untranslated RNA (scoutRNA), an associated trans-activating RNA (tracrRNA) or a combination thereof. The combination of a crRNA with a tracrRNA may be referred to herein as a single guide RNA (sgRNA), wherein the crRNA and the tracrRNA are covalently linked. In some embodiments, the crRNA and tracrRNA are linked by a phosphodiester bond. In some instances, the crRNA and tracrRNA are linked by one or more linked nucleotides. A guide nucleic acid may comprise a naturally occurring guide nucleic acid. A guide nucleic acid may comprise a non-naturally occurring guide nucleic acid, including a guide nucleic acid that is designed to contain a chemical or biochemical modification.

In general, the crRNA comprises a spacer region that hybridizes to a target sequence of a target nucleic acid, and a repeat region that interacts with the effector protein. The spacer region may comprise complementarity with (e.g., hybridize to) a target sequence of a target nucleic acid. In some cases, the spacer region is 15-28 linked nucleosides in length. In some cases, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some cases, the spacer region is 18-24 linked nucleosides in length. In some cases, the spacer region is at least 15 linked nucleosides in length. In some cases, the spacer region is at least 16, 18, 20, or 22 linked nucleosides in length. In some cases, the spacer region comprises at least 10, 11, 12, 13, 14, 15, 16, 17, 18, 19, 20, 21, 22, 23, 24, 25, 26, 27, 28, 29, or 30 nucleotides. In some cases, the spacer region is at least 17 linked nucleosides in length. In some cases, the spacer region is at least 18 linked nucleosides in length. In some cases, the spacer region is at least 20 linked nucleosides in length. In some cases, the spacer region is at least 80%, at least 85%, at least 90%, at least 95% or 100% complementary to a target sequence of the target nucleic acid. In some cases, the spacer region is 100% complementary to the target sequence of the target nucleic acid. In some cases, the spacer region comprises at least 15 contiguous nucleobases that are complementary to the target nucleic acid. The repeat region may also be referred to as a “protein-binding segment.” Typically, the repeat region is adjacent to the spacer region. For example, a guide RNA that interacts with an effector protein comprises a repeat region that is 5′ of the spacer region.

It is understood that the sequence of a spacer region need not be 100% complementary to that of a target sequence of a target nucleic acid to hybridize or hybridize specifically to the target sequence. The engineered guide nucleic acid may comprise at least one uracil between nucleic acid residues 5 to 20 of the spacer region that is not complementary to the corresponding nucleoside of the target sequence. The engineered guide nucleic acid may comprise at least one uracil between nucleic acid residues 5 to 9, 10 to 14, or 15 to 20 of the spacer region that is not complementary to the corresponding nucleoside of the target sequence. In some cases, the region of the target nucleic acid that is complementary to the spacer region comprises an epigenetic modification or a post-transcriptional modification. In some cases, the epigenetic modification comprises an acetylation, methylation, or thiol modification.

In some instances, the guide RNA comprises a tracrRNA. In some instances, the tracrRNA comprises a stem-loop structure comprising a stem region and a loop region. In some cases, the stem region is 4 to 8 linked nucleosides in length. In some cases, the stem region is 5 to 6 linked nucleosides in length. In some cases, the stem region is 4 to 5 linked nucleosides in length. In some cases, the tracrRNA comprises a pseudoknot (e.g., a secondary structure comprising a stem at least partially hybridized to a second stem or half-stem secondary structure). An effector protein may recognize a tracrRNA comprising multiple stem regions. In some instances, the amino acid sequences of the multiple stem regions are identical to one another. In some instances, the amino acid sequences of at least one of the multiple stem regions is not identical to those of the others. In some cases, the tracrRNA comprises at least 2, at least 3, at least 4, or at least 5 stem regions. A tracrRNA may include deoxyribonucleosides, ribonucleosides, chemically modified nucleosides, or any combination thereof. A tracrRNA may be separate from, but form a complex with, a guide nucleic acid and an effector protein. The tracrRNA may be attached (e.g., covalently) by an artificial linker to a guide nucleic acid. A tracrRNA may include a nucleotide sequence that hybridizes with a portion of a guide nucleic acid. A tracrRNA may also form a secondary structure (e.g., one or more hairpin loops) that facilitates the binding of an effector protein to a guide nucleic acid and/or modification activity of an effector protein on a target nucleic acid. A tracrRNA may include a repeat hybridization region and a hairpin region. The repeat hybridization region may hybridize to all or part of the repeat sequence of a guide nucleic acid. The repeat hybridization region may be positioned 3′ of the hairpin region. The hairpin region may include a first sequence, a second sequence that is reverse complementary to the first sequence, and a stem-loop linking the first sequence and the second sequence.

In some instances, the guide RNA does not comprise a tracrRNA. In some cases, an effector protein does not require a tracrRNA to locate and/or cleave a target nucleic acid. In some instances, the crRNA of the engineered guide nucleic acid comprises a repeat region and a spacer region, wherein the repeat region binds to the effector protein and the spacer region hybridizes to a target sequence of the target nucleic acid. The repeat sequence of the crRNA may interact with an effector protein, allowing for the engineered guide nucleic acid and the effector protein to form an RNP complex.

The guide RNA may bind to a target nucleic acid (e.g., a single strand of a target nucleic acid) or a portion thereof. The engineered guide nucleic acid may bind to a target nucleic acid such as a nucleic acid from a bacterium, a virus, a parasite, a protozoon, a fungus or other agents responsible for a disease, or an amplicon thereof. The target nucleic acid may comprise a mutation, such as a single nucleotide polymorphism (SNP), a chromosomal mutation, a copy number mutation, or any combination thereof. A point mutation optionally comprises a substitution, insertion, or deletion. In some embodiments, a mutation comprises a chromosomal mutation. A chromosomal mutation can comprise an inversion, a deletion, a duplication, or a translocation. In some embodiments, a mutation comprises a copy number variation. A copy number variation can comprise a gene amplification or an expanding trinucleotide repeat. A mutation may confer for example, resistance to a treatment, such as antibiotic treatment. The engineered guide nucleic acid may bind to a target nucleic acid, such as DNA or RNA, from a cancer gene or gene associated with a genetic disorder, or an amplicon thereof, as described herein. The target nucleic acid may be from any organism, including, but not limited to, a bacterium, a virus, a parasite, a protozoon, a fungus, a mammal, a plant, and an insect. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

In some cases, an effector protein cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some cases, a repeat region of a guide RNA comprises mutations or truncations relative to respective regions in a corresponding pre-crRNA.

The engineered guide nucleic acid may comprise a first region complementary to a target nucleic acid (FR1) and a second region that is not complementary to the target nucleic acid (FR2). In some cases, FRI is located 5′ to FR2 (FR1-FR2). In some cases, FR2 is located 5′ to FRI (FR2-FR1).

In some cases, the engineered guide nucleic acid comprises 10, 11, 12, 13, 14, 15, 16, 17, 18, 19, 20, 21, 22, 23, 24, 25, 26, 27, 28, 29, or 30 linked nucleosides. In general, an engineered guide nucleic acid comprises at least linked nucleosides. In some instances, an engineered guide nucleic acid comprises at least 25 linked nucleosides. An engineered guide nucleic acid may comprise 10 to 50 linked nucleosides. In some cases, the engineered guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the engineered guide nucleic acid has about 10 to about 60, about 20 to about 50, or about 30 to about 40 linked nucleosides.

### Certain Engineered Guide Nucleic Acids

An engineered guide nucleic acid of the compositions and systems described herein may comprise a crRNA, wherein the crRNA comprises a nucleobase sequence that is at least 70%, at least 75%, at least 80%, at least 85%, at least 90%, at least 95%, at least 97%, or 100% identical to any one of SEQ ID NOs: 19-21, as provided in TABLE 2. In some instances, the nucleobase sequence of the crRNA is at least 75%, at least 80%, at least 85%, at least 90%, at least 95%, at least 97%, or 100% identical to any one of SEQ ID NOs: 19-21. In some instances, the nucleobase sequence of the crRNA is at least 75%, at least 80%, at least 85%, at least 90%, at least 95%, at least 97%, or 100% identical to any one of SEQ ID NOs: 52-54.

TABLE 2
Nucleobase Sequences for crRNA

| SEQ ID NO | Sequence |
|---|---|
| SEQ ID NO: 19 | AGAUUUCUACUUUUGUAGAU |
| | UAUUAAAUACUCGUAUUGCU |
| SEQ ID NO: 20 | GAAUUUCUACUAUUGUAGAU |
| | UAUUAAAUACUCGUAUUGCU |
| SEQ ID NO: 21 | AGAUUUCUACUAUUGUAGAU |
| | UAUUAAAUACUCGUAUUGCU |
| SEQ ID NO: 52 | GAAUUUCUACUAUUGUAGAU |
| | GCCGAUAAUGAUGUAGGGAU |
| SEQ ID NO: 53 | UAAUUUCUACUAAGUGUAGA |
| | UGCCGAUAAUGAUGUAGGGA |
| | U |
| SEQ ID NO: 54 | UAAUUUCUACUAAGUGUAGA |
| | UCCCCCAGCGCUUCAGCGUU |
| | C |

In some cases, an engineered guide nucleic acid comprises a nucleobase sequence that is at least 75%, at least 80%, at least 85%, at least 90%, at least 95%, at least 98%, at least 99%, or 100% identical to GAAUUUCUACUAUUGUAGAU (SEQ ID NO: 55) or UAAUUUCUACUAAGUGUAGAU (SEQ ID NO: 56). In some cases, an engineered guide nucleic acid comprises the nucleobase sequence of GAAUUUCUACUAUUGUAGAU (SEQ ID NO: 55) or UAAUUUCUACUAAGUGUAGAU (SEQ ID NO: 56).

In some cases, an engineered guide nucleic acid comprises a repeat sequence comprising a nucleobase sequence that is at least 75%, at least 80%, at least 85%, at least 90%, at least 95%, at least 98%, at least 99%, or 100% identical to the underlined portion of SEQ ID NO: 19, 20, 21, 52, 53, or 54 shown in Table 2.

Provided herein are compositions comprising an engineered guide nucleic acid and an effector protein. The engineered guide nucleic acid and the effector protein may be capable of binding and/or forming a complex. The effector protein may comprise an amino acid sequence that is at least 75%, at least 80%, at least 85%, at least 90%, at least 95%, at least 99% or 100% identical to SEQ ID NO: 1, and an engineered guide nucleic acid comprising a nucleobase sequence that is at least 75%, at least 80%, at least 85%, at least 90%, at least 95%, at least 97%, or 100% identical to SEQ ID NO: 19. The amino acid sequence of the effector protein may be at least 80%, at least 85%, at least 90%, at least 95%, at least 99% or 100% identical to SEQ ID NO: 1, and the nucleobase sequence of the engineered guide nucleic acid may be at least 75%, at least 80%, at least 85%, at least 90%, at least 95%, at least 97%, or 100% identical to SEQ ID NO: 19. The amino acid sequence of the effector protein may be at least 80%, at least 85%, at least 90%, at least 95%, at least 99% or 100% identical to SEQ ID NO: 2, and the nucleobase sequence of the engineered guide nucleic acid may be at least 75%, at least 80%, at least 85%, at least 90%, at least 95%, at least 97%, or 100% identical to SEQ ID NO: 20. The amino acid sequence of the effector protein may be at least 80%, at least 85%, at least 90%, at least 95%, at least 99% or 100% identical to SEQ ID NO: 3, and the nucleobase sequence of the engineered guide nucleic acid may be at least 75%, at least 80%, at least 85%, at least 90%, at least 95%, at least 97%, or 100% identical to SEQ ID NO: 19. The amino acid sequence of the effector protein may be at least 80%, at least 85%, at least 90%, at least 95%, at least 99% or 100% identical to SEQ ID NO: 4, and the nucleobase sequence of the engineered guide nucleic acid may be at least 75%, at least 80%, at least 85%, at least 90%, at least 95%, at least 97%, or 100% identical to SEQ ID NO: 21.

Provided herein are compositions comprising an effector protein and an engineered guide nucleic acid, wherein the effector protein provides transcollateral cleavage activity upon binding of the engineered guide nucleic acid to a target nucleic acid at a temperature within a range of about 45° C. to 80° C. In some cases, the effector protein comprises an amino acid sequence that is at least 75%, at least 80%, at least 85%, at least 90%, at least 95%, at least 98%, at least 99%, or 100% identical to SEQ ID NO: 2. In some cases, the composition further comprises Mg²⁺. In some cases, the composition has a pH within a range of about 8.0 to 9.0. In some cases, the composition further comprises a reporter nucleic acid. In some cases, the composition has a temperature of at least 45° C. In some cases, the effector protein provides higher transcollateral cleavage activity at 40, 45, 50, 55, 60, 65, or 70° C. than at 37° C. when the target nucleic acid comprises a single nucleotide polymorphism (SNP). In some cases, the effector protein provides higher transcollateral cleavage activity at 70° C. than at 37° C. when the target nucleic acid comprises a single nucleotide polymorphism (SNP). In some cases, the effector protein has a threshold of detection of less than 250 pM, less than 25 pM, less than 2.5 pM, or less than 250 fM of the target nucleic acid at a temperature within a range of about 45° C. to 80° C. In some cases, the target nucleic acid is present in a sample at a concentration of less than 250 pM, less than 25 pM, less than 2.5 pM, or less than 250 fM. In some cases, the effector protein has catalytic efficiency of at least about 1×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2×10⁷, 3×10⁷, 4×10⁷, or 5×10 M⁻¹s⁻¹ at a temperature of about 45, 50, 55, 60, 65, 70, 75, or 80° C. In some cases, the effector protein has catalytic efficiency of at least about 1.7×10⁷ M⁻¹s⁻¹ at a temperature within a range of about 45° C. to 80° C.

Provided herein are complexes comprising an engineered guide nucleic acid and an effector protein described herein. Complexes comprising an engineered guide nucleic acid and an effector protein described herein are capable of binding to a target sequence.

A complex may comprise an effector protein comprising an amino acid sequence that is at least 75%, at least 80%, at least 85%, at least 90%, at least 95%, at least 99% or 100% identical to SEQ ID NO: 1, and an engineered guide nucleic acid comprising a nucleobase sequence that is at least 75%, at least 80%, at least 85%, at least 90%, at least 95%, at least 97%, or 100% identical to SEQ ID NO: 19. The amino acid sequence of the effector protein may be at least 80%, at least 85%, at least 90%, at least 95%, at least 99% or 100% identical to SEQ ID NO: 1, and the nucleobase sequence of the engineered guide nucleic acid may be at least 75%, at least 80%, at least 85%, at least 90%, at least 95%, at least 97%, or 100% identical to SEQ ID NO: 19.

A complex may comprise an effector protein comprising an amino acid sequence that is at least 75%, at least 80%, at least 85%, at least 90%, at least 95%, at least 99% or 100% identical to SEQ ID NO: 2, and an engineered guide nucleic acid comprising a nucleobase sequence that is at least 75%, at least 80%, at least 85%, at least 90%, at least 95%, at least 97%, or 100% identical to SEQ ID NO: 20.

A complex may comprise an effector protein comprising an amino acid sequence that is at least 75%, at least 80%, at least 85%, at least 90%, at least 95%, at least 99% or 100% identical to SEQ ID NO: 3, and an engineered guide nucleic acid comprising a nucleobase sequence that is at least 75%, at least 80%, at least 85%, at least 90%, at least 95%, at least 97%, or 100% identical to SEQ ID NO: 19.

A complex may comprise an effector protein comprising an amino acid sequence that is at least 75%, at least 80%, at least 85%, at least 90%, at least 95%, at least 99% or 100% identical to SEQ ID NO: 4, and an engineered guide nucleic acid comprising a nucleobase sequence that is at least 75%, at least 80%, at least 85%, at least 90%, at least 95%, at least 97%, or 100% identical to SEQ ID NO: 21

Provided herein are vectors encoding an engineered guide nucleic acid disclosed herein.

In some cases, a vector encodes an engineered guide comprises a nucleobase sequence that is at least 75%, at least 80%, at least 85%, at least 90%, at least 95%, at least 97%, or 100% identical to SEQ ID NO: 19, SEQ ID NO: 20 or SEQ ID NO: 21.

In some cases, a vector encodes an effector protein described herein and an engineered guide comprises a nucleobase sequence that is at least 75%, at least 80%, at least 85%, at least 90%, at least 95%, at least 97%, or 100% identical to SEQ ID NO: 19, SEQ ID NO: 20 or SEQ ID NO: 21.

In some cases, a vector encodes an effector protein comprising at least 75%, at least 80%, at least 85%, at least 90%, at least 95%, at least 99% or 100% identical to SEQ ID NO: 1, and an engineered guide nucleic acid comprising a nucleobase sequence that is at least 75%, at least 80%, at least 85%, at least 90%, at least 95%, at least 97%, or 100% identical to SEQ ID NO: 19.

In some cases, a vector system comprises a first vector that encodes an effector protein comprising at least 75%, at least 80%, at least 85%, at least 90%, at least 95%, at least 99% or 100% identical to SEQ ID NO: 1, and a second vector that encodes an engineered guide nucleic acid comprising a nucleobase sequence that is at least 75%, at least 80%, at least 85%, at least 90%, at least 95%, at least 97%, or 100% identical to SEQ ID NO: 19.

In some cases, a vector encodes an effector protein comprising at least 75%, at least 80%, at least 85%, at least 90%, at least 95%, at least 99% or 100% identical to SEQ ID NO: 2, and an engineered guide nucleic acid comprising a nucleobase sequence that is at least 75%, at least 80%, at least 85%, at least 90%, at least 95%, at least 97%, or 100% identical to SEQ ID NO: 20.

In some cases, a vector system comprises a first vector that encodes an effector protein comprising at least 75%, at least 80%, at least 85%, at least 90%, at least 95%, at least 99% or 100% identical to SEQ ID NO: 2, and a second vector that encodes an engineered guide nucleic acid comprising a nucleobase sequence that is at least 75%, at least 80%, at least 85%, at least 90%, at least 95%, at least 97%, or 100% identical to SEQ ID NO: 20.

In some cases, a vector encodes an effector protein comprising an amino acid sequence that is at least 75%, at least 80%, at least 85%, at least 90%, at least 95%, at least 99% or 100% identical to SEQ ID NO: 3, and an engineered guide nucleic acid comprising a nucleobase sequence that is at least 75%, at least 80%, at least 85%, at least 90%, at least 95%, at least 97%, or 100% identical to SEQ ID NO: 19.

In some cases, a vector system comprises a first vector that encodes an effector protein comprising at least 75%, at least 80%, at least 85%, at least 90%, at least 95%, at least 99% or 100% identical to SEQ ID NO: 3, and a second vector that encodes an engineered guide nucleic acid comprising a nucleobase sequence that is at least 75%, at least 80%, at least 85%, at least 90%, at least 95%, at least 97%, or 100% identical to SEQ ID NO: 19.

In some cases, a vector encodes an effector protein comprising an amino acid sequence that is at least 75%, at least 80%, at least 85%, at least 90%, at least 95%, at least 99% or 100% identical to SEQ ID NO: 4, and an engineered guide nucleic acid comprising a nucleobase sequence that is at least 75%, at least 80%, at least 85%, at least 90%, at least 95%, at least 97%, or 100% identical to SEQ ID NO: 21.

In some cases, a vector system comprises a first vector that encodes an effector protein comprising at least 75%, at least 80%, at least 85%, at least 90%, at least 95%, at least 99% or 100% identical to SEQ ID NO: 4, and a second vector that encodes an engineered guide nucleic acid comprising a nucleobase sequence that is at least 75%, at least 80%, at least 85%, at least 90%, at least 95%, at least 97%, or 100% identical to SEQ ID NO: 21.

In some cases, the vectors disclosed herein comprise codon optimized sequences that encode an effector protein disclosed herein.

### A. Pooling Guide Nucleic Acids

In some instances, compositions, systems or methods provided herein comprise a pool of guide nucleic acids. In some instances, the pool of guide nucleic acids were tiled against a target nucleic acid, e.g., the genomic locus of interest or uses thereof. In some instances, an engineered guide nucleic acid is selected from a group of guide nucleic acids that have been tiled against a nucleic acid sequence of a genomic locus of interest. The genomic locus of interest may belong to a viral genome, a bacterial genome, or a mammalian genome. Non-limiting examples of viral genomes are an HPV genome, an HIV genome, an influenza genome, or a coronavirus genome. Often, these guide nucleic acids are pooled for detecting a target nucleic acid in a single assay. Pooling of guide nucleic acids may ensure broad spectrum identification, or broad coverage, of a target species within a single reaction. This may be particularly helpful in diseases or indications, like sepsis, that may be caused by multiple organisms. The pool of guide nucleic acids may enhance the detection of a target nucleic using systems of methods described herein relative to detection with a single guide nucleic acid. The pool of guide nucleic acids may ensure broad coverage of the target nucleic acid within a single reaction using the methods described herein. In some instances, the pool of guide nucleic acids are collectively complementary to at least 50%, at least 55%, at least 60%, at least 65%, at least 70%, at least 75%, at least 80%, at least 85%, at least 90%, at least 95%, at least 97%, or 100% of the target nucleic acid. In some instances, at least a portion of the engineered guide nucleic acids of the pool overlap in sequence. In some instances, at least a portion of the engineered guide nucleic acids of the pool do not overlap in sequence. In some cases, the pool of guide nucleic acids comprises at least 2, at least 3, at least 4, at least 5, or at least 6 guide nucleic acids targeting different sequences of a target nucleic acid.

### B. Intermediary Nucleic Acids

An engineered guide nucleic acid may comprise or be coupled to an intermediary nucleic acid. The intermediary nucleic acid may also be referred to as an intermediary RNA, although it may comprise deoxyribonucleosides in addition to ribonucleosides. The intermediary RNA may be separate from, but form a complex with a crRNA to form a discrete gRNA system. The intermediary RNA may be linked to a crRNA to form a composite gRNA. An effector protein may bind a crRNA and an intermediary RNA. In some cases, the crRNA and the intermediary RNA are provided as a single nucleic acid (e.g., covalently linked). In some embodiments, the crRNA and the intermediary RNA are separate polynucleotides (e.g., a discrete gRNA system). An intermediary RNA may comprise a repeat hybridization region and a hairpin region. The repeat hybridization region may hybridize to all or part of the sequence of the repeat of a crRNA. The repeat hybridization region may be positioned 3′ of the hairpin region. The hairpin region may comprise a first sequence, a second sequence that is reverse complementary to the first sequence, and a stem-loop linking the first sequence and the second sequence.

The CRISPR/Cas ribonucleoprotein (RNP) complex may comprise a Cas protein complexed with an engineered guide nucleic acid (e.g., a crRNA) and an intermediary RNA. Sometimes, an engineered guide nucleic acid comprises a crRNA and an intermediary RNA (e.g., the crRNA and intermediary RNA are provided as a single nucleic acid molecule). A composition comprises a crRNA, an intermediary RNA, a Cas protein, and a detector nucleic acid.

In some instances, the length of intermediary RNAs is not greater than 50, 56, 68, 71, 73, 95, or 105 linked nucleosides. In some embodiments, the length of an intermediary RNA is about 30 to about 120 linked nucleosides. In some embodiments, the length of an intermediary RNA is about 50 to about 105, about 50 to about 95, about 50 to about 73, about 50 to about 71, about 50 to about 68, or about 50 to about 56 linked nucleosides. In some embodiments, the length of an intermediary RNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some embodiments, the length of an intermediary RNA is 40 to 60 nucleotides. In some embodiments, the length of the intermediary RNA is 50, 56, 68, 71, 73, 95, or 105 linked nucleosides. In some embodiments, the length of the intermediary RNA is 50 nucleotides.

An exemplary intermediary RNA may comprise, from 5′ to 3′, a 5′ region, a hairpin region, a repeat hybridization region, and a 3′ region. In some cases, the 5′ region may hybridize to the 3′ region. In some embodiments, the 5′ region does not hybridize to the 3′ region. In some cases, the 3′ region is covalently linked to the crRNA (e.g., through a phosphodiester bond). In some embodiments, an intermediary RNA may comprise an un-hybridized region at the 3′ end of the intermediary RNA. The un-hybridized region may have a length of about 1, about 2, about 3, about 4, about 5, about 6, about 7, about 8, about 9, about 10, about 12, about 14, about 16, about 18, or about 20 linked nucleosides. In some embodiments, the length of the un-hybridized region is 0 to 20 linked nucleosides.

### III. Modifications

Polypeptides (e.g., effector proteins) and nucleic acids (e.g., engineered guide nucleic acids) described herein can be further modified as described throughout and as further described herein.

Examples are modifications of interest that do not alter primary sequence, including chemical derivatization of polypeptides, e.g., acylation, acetylation, carboxylation, amidation, etc. Also included are modifications of glycosylation, e.g., those made by modifying the glycosylation patterns of a polypeptide during its synthesis and processing or in further processing steps; e.g., by exposing the polypeptide to enzymes which affect glycosylation, such as mammalian glycosylating or deglycosylating enzymes. Also embraced are sequences that have phosphorylated amino acid residues, e.g., phosphotyrosine, phosphoserine, or phosphothreonine.

Modifications disclosed herein can also include modification of described polypeptides and/or engineered guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g., D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or engineered guide nucleic acids described herein. For example, groups can be introduced during synthesis or during expression of a polypeptide (e.g., a programmable nuclease), which allow for linking to other molecules or to a surface. Thus, e.g., cysteines can be used to make thioethers, histidines for linking to a metal ion complex, carboxyl groups for forming amides or esters, amino groups for forming amides, and the like.

Modifications can further include modification of nucleic acids described herein (e.g., engineered guide nucleic acids) to provide the nucleic acid with a new or enhanced feature, such as improved stability. Such modifications of a nucleic acid include a base modification, a backbone modification, a sugar modification, or combinations thereof, of one or more nucleotides, nucleosides, or nucleobases in a nucleic acid.

In some embodiments, nucleic acids (e.g., engineered guide nucleic acids) described herein comprise one or more modifications comprising: 2′O-methyl modified nucleotides, 2′ Fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2—NH—O—CH2—, —CH2—N(CH3)—O—CH2— (known as a methylene (methylimino) or MMI backbone), —CH2—O—N(CH3)—CH2—, —CH2—N(CH3)—N(CH3)—CH2— and —O—N(CH3)—CH2—CH2—(wherein the native phosphodiester internucleotide linkage is represented as —O—P(═O)(OH)—O—CH2—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

### IV. Systems

Disclosed herein, in some aspects, are systems for modifying a nucleic acid, comprising any one of the effector proteins described herein, a fusion protein thereof, or a multimeric complex thereof. Systems may be used to detect, modify, or edit a target nucleic acid. Systems may be used to modify the activity or expression of a target nucleic acid. In some embodiments, systems comprise an effector protein described herein, an engineered guide nucleic acid, a reagent, a support medium, or a combination thereof. In some instances, the effector protein comprises an amino acid sequence that is at least 70%, at least 75%, at least 80%, at least 85%, at least 90%, at least 95%, or 100% identical to any one of SEQ ID NOs: 1-4. In some instances, the amino acid sequence of the effector protein is at least 70%, at least 75%, at least 80%, at least 85%, at least 90%, at least 95%, or 100% identical to any one of SEQ ID NOs: 1-4.

Systems may be used for detecting the presence of a target nucleic acid associated with or causative of a disease, such as cancer, a genetic disorder, or an infection. In some instances, systems are useful for phenotyping, genotyping, or determining ancestry. Unless specified otherwise, systems include kits and may be referred to as kits. Unless specified otherwise, systems include devices and may also be referred to as devices. Systems may be provided in the form of a companion diagnostic assay or device, a point-of-care assay or device, or an over-the-counter diagnostic assay/device.

Reagents and effector proteins of various systems may be provided in a reagent chamber or on the support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

In some embodiments, the reagent chamber and/or support medium may be provided in or on a device.

Often, systems comprise a temperature modulator. The temperature modulator may increase, decrease or maintain the temperature of system components, system reagents, samples, and compositions disclosed herein. Non-limiting examples of temperature modulators are wires, electrodes, and heating plates. The temperature modulator may be connected to the system. The temperature modulator may be separate from the system. The temperature modulator may be capable of heating system components, system reagents, samples, compositions, or combinations thereof to at least about 40° C., at least about 45° C., at least about 50° C., at least about 55° C., at least about 60° C., at least about 65° C., at least about 70° C., at least about 75° C., or at least about 80° C. The temperature modulator may be capable of heating system components, system reagents, samples, compositions, or combinations thereof to about 40° C., about 45° C., about 50° C., about 55° C., about 60° C., about 65° C., about 70° C., about 75° C., or about 80° C.

### A. Certain System Components and Reagents

### System Solutions

In general, systems comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. The solution or buffer may comprise a buffering agent. The solution or buffer may comprise a salt. The solution or buffer may comprise a crowding agent. The solution or buffer may comprise a detergent. The solution or buffer may comprise a reducing agent. The solution or buffer may comprise a competitor. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some instances, a buffer is required for cell lysis activity or viral lysis activity.

In some cases, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some instances, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, 7 to 9, 7 to 9.5, 6.5 to 8, 6.5 to 9, 6.5 to 9.5, 7.5 to 8.5, 7.5 to 9, 7.5 to 9.5, or 9.5 to 10.5. The pH of the solution may also be at least about 6.0, at least about 6.5, at least about 7.0, at least about 7.5, at least about 8.0, at least about 8.5, or at least about 9. In some cases, the pH is at least about 6. In some cases, the pH is at least about 6.5. In some cases, the pH is at least about 7. In some cases, the pH is at least about 7.5. In some cases, the pH is at least about 8. In some cases, the pH is at least about 8.5. In some cases, the pH is at least about 9. In some cases, the solution is basic. The pH of a basic solution is more than 7. In some cases, the pH of a basic solution is more than 7.5.

In some cases, a buffer may comprise a catalytic reagent for signal improvement or enhancement. In some cases, the catalytic reagent may enhance signal generation via hydrolysis of inorganic pyrophosphates. In some cases, the catalytic reagent may enhance signal generation via enhancement of DNA replication. In some cases, the catalytic reagent may enhance signal amplification via revival of Mg²⁺ ions in the buffer solution which may otherwise be taken up by the phosphates produced from usage of dNTPs during the LAMP or NEAR reaction (or other isothermal or thermocycling replication reaction described herein). In some cases, the catalytic reagent may enhance signal generation by reviving the concentration of Mg²⁺ ions in the buffer thereby enhancing the function of the Cas nuclease effector enzyme. In some cases, the catalytic reagent for signal improvement may be an enzyme. In some cases, the catalytic reagent for signal improvement may be a Thermostable Inorganic Pyrophosphatase (TIPP).

In some cases, a buffer used in the compositions and methods disclosed herein does not comprise TIPP.

In some cases, systems comprise a solution, wherein the solution comprises at least one salt. In some instances, the at least one salt is selected from potassium acetate, magnesium acetate, sodium chloride, potassium chloride, magnesium chloride, calcium chloride, and any combination thereof. In some instances, the concentration of the at least one salt in the solution is 5 millimolar (mM) to 100 mM, 5 mM to 10 mM, 1 mM to 60 mM, or 1 mM to 10 mM. In some instances, the concentration of the at least one salt is about 105 mM. In some instances, the concentration of the at least one salt is about 55 mM. In some instances, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some instances, the salt is a magnesium salt and the concentration of magnesium in the solution is at least 5 mM, at least 7 mM, at least 9 mM, at least 11 mM, at least 13 mM, or at least 15 mM. In some instances, the concentration of magnesium is less than 20 mM, less than 18 mM or less than 16 mM.

In some instances, the salt is a magnesium salt, a potassium salt, a sodium salt, or a calcium salt. In some instances, the salt is a magnesium salt. In some instances, the salt is a potassium salt. In some instances, the salt is a sodium salt. In some instances, the salt is a calcium salt. In some instances, the salt in the solution is at least about 1 mM, at least about 3 mM, at least about 5 mM, at least about 7 mM, at least about 9 mM, at least about 11 mM, at least about 13 mM, or at least about 15 mM. In some instances, the salt in the solution is at least about 1 mM. In some instances, the salt in the solution is at least about 2 mM. In some instances, the salt in the solution is at least about 3 mM. In some instances, the salt in the solution is at least about 4 mM. In some instances, the salt in the solution is at least about 5 mM. In some instances, the salt in the solution is at least about 6 mM. In some instances, the salt in the solution is at least about 7 mM. In some instances, the salt in the solution is at least about 8 mM. In some instances, the salt in the solution is at least about 9 mM. In some instances, the salt in the solution is at least about 10 mM. In some instances, the salt in the solution is at least about 11 mM. In some instances, the salt in the solution is at least about 12 mM. In some instances, the salt in the solution is at least about 13 mM. In some instances, the salt in the solution is at least about 14 mM. In some instances, the salt in the solution is at least about 15 mM. In some instances, the salt in the solution is at least about 16 mM. In some instances, the salt in the solution is at least about 17 mM. In some instances, the salt in the solution is at least about 18 mM. In some instances, the salt in the solution is at least about 19 mM. In some instances, the salt in the solution is at least about 20 mM. In some instances, the salt in the solution is at least about 0.00001 mM, 0.00005 mM, 0.0001 mM, 0.0005 mM, 0.001 mM, 0.005 mM, 0.01 mM, 0.05 mM, 0.1 mM, or 0.5 mM. In some instances, the salt in the solution is from 0.00001 mM to 0.0001 mM, from 0.00005 mM to 0.0005 mM, from 0.0001 mM to 0.001 mM, from 0.0005 mM to 0.005 mM, from 0.001 mM to 0.01 mM, from 0.005 mM to 0.05 mM, from 0.01 mM to 0.1 mM, from 0.05 mM to 0.5 mM, from 0.1 mM to 1 mM, from 0.5 mM to 1.5 mM, from 1 mM to 2 mM, from 1.5 mM to 2.5 mM, from 2 mM to 3 mM, from 2.5 mM to 3.5 mM, from 3 mM to 4 mM, from 3.5 mM to 4.5 mM, from 4 mM to 5 mM, from 4.5 mM to 5.5 mM, from 5 mM to 6 mM, from 5.5 mM to 6.5 mM, from 6 mM to 7 mM, from 6.5 mM to 7.5 mM, from 7 mM to 8 mM, from 7.5 mM to 8.5 mM, from 8 mM to 9 mM, from 8.5 mM to 9.5 mM, from 9 mM to 10 mM, from 9.5 mM to 10.5 mM, from 10 mM to 11 mM, from 10.5 mM to 11.5 mM, from 11 mM to 12 mM, from 11.5 mM to 12.5 mM, from 12 mM to 13 mM, from 12.5 mM to 13.5 mM, from 13 mM to 14 mM, from 13.5 mM to 14.5 mM, from 14 mM to 15 mM, from 14.5 mM to 15.5 mM, from 15 mM to 16 mM, from 15.5 mM to 16.5 mM, from 16 mM to 17 mM, from 16.5 mM to 17.5 mM, from 17 mM to 18 mM, from 17.5 mM to 18.5 mM, from 18 mM to 19 mM, from 18.5 mM to 19.5 mM, or from 19 mM to 20 mM.

In some embodiments, the solution comprises a range from about 15 mM to about 20 mM of a divalent salt (e.g., Mg²⁺) at a pH range from about 6 to about 9.

In some cases, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some instances, the crowding agent is glycerol. In some instances, the concentration of the crowding agent in the solution is 0.01% (v/v) to 10% (v/v). In some instances, the concentration of the crowding agent in the solution is 0.5% (v/v) to 10% (v/v).

In some cases, systems comprise a solution, wherein the solution comprises at least one simple sugar or sugar alcohol. The presence of simple sugars or sugar alcohols advantageously increases trans cleavage by effector proteins. Exemplary simple sugars include sucrose, and trehalose. An exemplary sugar alcohol is xylitol. In some instances, the sugar alcohol is xylitol. In some instances, the simple sugar is sucrose. In some instances, the simple sugar is trehalose.

In some cases, systems comprise a solution, wherein the solution comprises bovine serum albumin (BSA). In some cases, the BSA is recombinant BSA. The presence of BSA advantageously increases trans cleavage by effector proteins. In some cases, the concentration of the BSA in the solution is 10 μg/ml to 1000 μg/ml, 10 μg/ml to 500 μg/ml, 50 μg/ml to 1000 μg/ml, 50 μg/ml to 500 μg/ml, or 50 μg/ml to 250 μg/ml. In preferred cases, the concentration of the BSA in the solution is 50 μg/ml to 250 μg/ml.

In some cases, systems comprise a solution, wherein the solution comprises at least one detergent (e.g. a non-ionic detergent). Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some cases, systems comprise a solution, wherein the solution comprises at least one reducing agent. Exemplary reducing agents comprise dithiothreitol (DTT), β-mercaptoethanol (BME), or tris(2-carboxyethyl)phosphine (TCEP). In some instances, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some cases, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some cases, the concentration of the competitor in the solution is 1 μg/mL to 100 μg/mL. In some cases, the concentration of the competitor in the solution is 40 μg/mL to 60 μg/mL.

In some cases, systems comprise a solution, wherein the solution comprises a co-factor. In some cases, the co-factor allows an effector protein to perform a function, including pre-crRNA processing and/or target nucleic acid cleavage. For example, as discussed in Jiang F. and Doudna J. A. (Annu. Rev. Biophys. 2017. 46:505-29), Cas9 may use divalent metal ions as co-factors. The suitability of a cofactor for an effector protein may be assessed, such as by methods based on those described by Sundaresan et al. (Cell Rep. 2017 Dec. 26; 21(13): 3728-3739). In some cases, a programmable or a multimeric complex thereof forms a complex with a co-factor. In some cases, the co-factor is a divalent metal ion. In some embodiments, the divalent metal ion is selected from Mg²⁺, Mn²⁺, Zn²⁺, Ca²⁺, Cu²⁺. In some cases, the divalent metal ion is Mg²⁺. In some cases, the effector protein is an effector protein and the co-factor is Mg²⁺+

### Reporters

In some instances, systems disclosed herein comprise a detection agent. The detection reagent may comprise a reporter nucleic acid, a detection moiety, an additional effector protein, or a combination thereof. The detection reagent may comprise a reporter nucleic acid. The detection reagent may comprise a detection moiety. The detection reagent may comprise an additional effector protein.

In some instances, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein, releasing the detection moiety, and, generating a detectable signal. As used herein, “reporter” is used interchangeably with “reporter nucleic acid” or “reporter molecule”. The effector proteins disclosed herein, activated upon hybridization of a guide RNA to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded. Reporters may comprise dsDNA. Reporters may comprise dsRNA. Reporters may comprise ssDNA. Reporters may comprise ssRNA.

In some instances, reporters comprise a protein capable of generating a signal. A signal may be a calorimetric, potentiometric, amperometric, optical (e.g., fluorescent, colorimetric, etc.), or piezo-electric signal. In some cases, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like. In some instances, reporters comprise a fluorophore, a quencher, or a combination thereof. In some instances, reporters comprise a fluorophore. In some instances, reporters comprise a quencher.

In some cases, the reporter comprises a detection moiety. In some instances, the reporter comprises a cleavage site, wherein the detection moiety is located at a first site on the reporter, wherein the first site is separated from the remainder of reporter upon cleavage at the cleavage site. In some cases, the detection moiety is 3′ to the cleavage site. In some cases, the detection moiety is 5′ to the cleavage site. Sometimes the detection moiety is at the 3′ terminus of the nucleic acid of a reporter. In some cases, the detection moiety is at the 5′ terminus of the nucleic acid of a reporter.

In some cases, the reporter comprises a detection moiety and a quenching moiety. In some instances, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some cases, the detection moiety is at the 5′ terminus of the nucleic acid of a reporter. In some cases, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some instances, the detection moiety comprises an invertase. The substrate of the invertase may be sucrose. A DNS reagent may be included in the system to produce a colorimetric change when the invertase converts sucrose to glucose. In some cases, the reporter nucleic acid and invertase are conjugated using a heterobifunctional linker via sulfo-SMCC chemistry.

Suitable fluorophores may provide a detectable fluorescence signal in the same range as 6-fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some cases, the fluorophore emits fluorescence at a wavelength of 700 nm or higher. In other cases, the fluorophore emits fluorescence at about 665 nm. In some cases, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some cases, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range 450 nm to 750 nm, 500 nm to 650 nm, or 550 to 650 nm. A quenching moiety may quench fluorescein amidite, 6-fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety indicates that cleavage by the effector proteins has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter. Other methods of detection can also be used, such as optical imaging, surface plasmon resonance (SPR), and/or interferometric sensing.

The detectable signal may be a colorimetric signal or a signal visible by eye. In some instances, the detectable signal may be fluorescent, electrical, chemical, electrochemical, or magnetic. In some cases, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some cases, the detectable signal may be generated directly by the cleavage event. Alternatively, or in combination, the detectable signal may be generated indirectly by the signal event. Sometimes the detectable signal is not a fluorescent signal. In some instances, the detectable signal may be a colorimetric or color-based signal. In some cases, the detected target nucleic acid may be identified based on its spatial location on the detection region of the support medium. In some cases, the second detectable signal may be generated in a spatially distinct location than the first detectable signal when two or more detectable signals are generated. Examples of reporter nucleic acids are provided in TABLE 3.

TABLE 3
Examples of Single Stranded Nucleic Acids in a Reporter

| 5′ Detection | | |
|---|---|---|
| Moiety* | Sequence (SEQ ID NO:) | 3′ Quencher* |
| /56-FAM/ | rUrUrUrUrU (SEQ ID NO: 22) | /3IABKFQ/ |
| /5IRD700/ | rUrUrUrUrU (SEQ ID NO: 22) | /3IRQCIN/ |
| /5TYE665/ | rUrUrUrUrU (SEQ ID NO: 22) | /3IAbRQSp/ |
| /5Alex594N/ | rUrUrUrUrU (SEQ ID NO: 22) | /3IAbRQSp/ |
| /5ATTO633N/ | rUrUrUrUrU (SEQ ID NO: 22) | /3IAbRQSp/ |
| /56-FAM/ | rUrUrUrUrUrUrUrU (SEQ ID NO: 23) | /3IABKFQ/ |
| /5IRD700/ | rUrUrUrUrUrUrUrU (SEQ ID NO: 23) | /3IRQCIN/ |
| /5TYE665/ | rUrUrUrUrUrUrUrU (SEQ ID NO: 23) | /3IAbRQSp/ |
| /5Alex594N/ | rUrUrUrUrUrUrUrU (SEQ ID NO: 23) | /3IAbRQSp/ |
| /5ATTO633N/ | rUrUrUrUrUrUrUrU (SEQ ID NO: 23) | /3IAbRQSp/ |
| /56-FAM/ | rUrUrUrUrUrUrUrUrUrU (SEQ ID NO: 24) | /3IABKFQ/ |
| /5IRD700/ | rUrUrUrUrUrUrUrUrUrU (SEQ ID NO: 24) | /3IRQC1N/ |
| /5TYE665/ | rUrUrUrUrUrUrUrUrUrU (SEQ ID NO: 24) | /3IAbRQSp/ |
| /5Alex594N/ | rUrUrUrUrUrUrUrUrUrU (SEQ ID NO: 24) | /3IAbRQSp/ |
| /5ATTO633N/ | rUrUrUrUrUrUrUrUrUrU (SEQ ID NO: 24) | /3IAbRQSp/ |
| /56-FAM/ | TTTTrUrUTTTT (SEQ ID NO: 25) | /3IABKFQ/ |
| /5IRD700/ | TTTTrUrUTTTT (SEQ ID NO: 25) | /3IRQC1N/ |
| /5TYE665/ | TTTTrUrUTTTT (SEQ ID NO: 25) | /3IAbRQSp/ |
| /5Alex594N/ | TTTTrUrUTTTT (SEQ ID NO: 25) | /3IAbRQSp/ |
| /5ATTO633N/ | TTTTrUrUTTTT (SEQ ID NO: 25) | /3IAbRQSp/ |
| /56-FAM/ | TTrUrUTT (SEQ ID NO: 26) | /3IABKFQ/ |
| /5IRD700/ | TTrUrUTT (SEQ ID NO: 26) | /3IRQC1N/ |
| /5TYE665/ | TTrUrUTT (SEQ ID NO: 26) | /3IAbRQSp/ |
| /5Alex594N/ | TTrUrUTT (SEQ ID NO: 26) | /3IAbRQSp/ |
| /5ATTO633N/ | TTrUrUTT (SEQ ID NO: 26) | /3IAbRQSp/ |
| /56-FAM/ | TArArUGC (SEQ ID NO: 27) | /3IABKFQ/ |
| /5IRD700/ | TArArUGC (SEQ ID NO: 27) | /3IRQCIN/ |
| /5TYE665/ | TArArUGC (SEQ ID NO: 27) | /3IAbRQSp/ |
| /5Alex594N/ | TArArUGC (SEQ ID NO: 27) | /3IAbRQSp/ |
| /5ATTO633N/ | TArArUGC (SEQ ID NO: 27) | /3IAbRQSp/ |
| /56-FAM/ | TArUrGGC (SEQ ID NO: 28) | /3IABKFQ/ |
| /5IRD700/ | TArUrGGC (SEQ ID NO: 28) | /3IRQC1N/ |
| /5TYE665/ | TArUrGGC (SEQ ID NO: 28) | /3IAbRQSp/ |
| /5Alex594N/ | TArUrGGC (SEQ ID NO: 28) | /3IAbRQSp/ |
| /5ATTO633N/ | TArUrGGC (SEQ ID NO: 28) | /3IAbRQSp/ |
| /56-FAM/ | rUrUrUrUrU (SEQ ID NO: 29) | /3IABKFQ/ |
| /5IRD700/ | rUrUrUrUrU (SEQ ID NO: 29) | /3IRQCIN/ |
| /5TYE665/ | rUrUrUrUrU (SEQ ID NO: 29) | /3IAbRQSp/ |
| /5Alex594N/ | rUrUrUrUrU (SEQ ID NO: 29) | /3IAbRQSp/ |
| /5ATTO633N/ | rUrUrUrUrU (SEQ ID NO: 29) | /3IAbRQSp/ |
| /56-FAM/ | TTATTATT (SEQ ID NO: 30) | /3IABKFQ/ |
| /56-FAM/ | TTATTATT (SEQ ID NO: 30) | /3IABKFQ/ |
| /5IRD700/ | TTATTATT (SEQ ID NO: 30) | /3IRQC1N/ |
| /5TYE665/ | TTATTATT (SEQ ID NO: 30) | /3IAbRQSp/ |
| /5Alex594N/ | TTATTATT (SEQ ID NO: 30) | /3IAbRQSp/ |
| /5ATTO633N/ | TTATTATT (SEQ ID NO: 30) | /3IAbRQSp/ |
| /56-FAM/ | TTTTTT (SEQ ID NO: 31) | /3IABKFQ/ |
| /56-FAM/ | TTTTTTTT (SEQ ID NO: 32) | /3IABKFQ/ |
| /56-FAM/ | TTTTTTTTTT (SEQ ID NO: 33) | /3IABKFQ/ |
| /56-FAM/ | TTTTTTTTTTTT (SEQ ID NO: 34) | /3IABKFQ/ |
| /56-FAM/ | TTTTTTTTTTTTTT (SEQ ID NO: 35) | /3IABKFQ/ |
| /56-FAM/ | AAAAAA (SEQ ID NO: 36) | /3IABKFQ/ |
| /56-FAM/ | CCCCCC (SEQ ID NO: 37) | /3IABKFQ/ |
| /56-FAM/ | GGGGGG (SEQ ID NO: 38) | /3IABKFQ/ |
| /56-FAM/ | TTATTATT (SEQ ID NO: 39) | /3IABKFQ/ |
| /56-FAM/ | CCCCCCCCCCCC (SEQ ID NO: 50) | /3IABKFQ/ |
| /5Alex594N/ | CCCCCCCCCCCC (SEQ ID NO: 50) | /3IAbRQSp/ |

/56-FAM/: 5′ 6-Fluorescein (Integrated DNA Technologies)
/3IABKFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQCIN/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

Often, the reporter is an enzyme-nucleic acid. The enzyme may be sterically hindered when present as in the enzyme-nucleic acid, but then functional upon cleavage from the nucleic acid by the programmable nuclease. Often, the enzyme is an enzyme that produces a reaction with an enzyme substrate. An enzyme can be invertase. Often, the substrate of invertase is sucrose and DNS reagent.

Sometimes the reporter is a substrate-nucleic acid. Often the substrate is a substrate that produces a reaction with an enzyme. Release of the substrate upon cleavage by the programmable nuclease may free the substrate to react with the enzyme.

A reporter may be attached to a solid support. The solid support, for example, is a surface. A surface can be an electrode. Sometimes the solid support is a bead. Often the bead is a magnetic bead. Upon cleavage, the detection moiety is liberated from the solid support and interacts with other mixtures. For example, the detection moiety is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the detection moiety is an enzyme substrate, and upon cleavage of the nucleic acid of the enzyme substrate-nucleic acid, the enzyme substrate flows through a chamber into a mixture comprising the enzyme. When the enzyme substrate meets the enzyme, a reaction occurs, such as a calorimetric reaction, which is then detected.

In some embodiments, the reporter comprises a nucleic acid conjugated to an affinity molecule which is in turn conjugated to the fluorophore (e.g., nucleic acid—affinity molecule—fluorophore) or the nucleic acid conjugated to the fluorophore which is in turn conjugated to the affinity molecule (e.g., nucleic acid—fluorophore—affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicates that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule.

In some cases, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

The reporter can be lyophilized or vitrified. The reporter can be suspended in solution or immobilized on a surface. For example, the reporter can be immobilized on the surface of a chamber in a device as disclosed herein. In some cases, the reporter is immobilized on beads, such as magnetic beads, in a chamber of a device as disclosed herein where they can be held in position by a magnet placed below the chamber.

In some cases, the reporter is a single-stranded nucleic acid comprising deoxyribonucleotides.

In some cases, the reporter nucleic acid is a single-stranded nucleic acid sequence comprising ribonucleotides. The nucleic acid of a reporter may be a single-stranded nucleic acid sequence comprising at least one ribonucleotide. In some cases, the nucleic acid of a reporter is a single-stranded nucleic acid comprising at least one ribonucleotide residue at an internal position that functions as a cleavage site. In some cases, the nucleic acid of a reporter comprises at least 2, at least 3, at least 4, at least 5, at least 6, at least 7, at least 8, at least 9, or at least 10 ribonucleotide residues at an internal position. In some cases, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the nucleic acid of a reporter has only ribonucleotide residues. In some cases, the nucleic acid of a reporter comprises at least one ribonucleotide residue and at least one non-ribonucleotide residue.

In some cases, the nucleic acid of a reporter comprises at least one uracil ribonucleotide. In some cases, the nucleic acid of a reporter comprises at least two uracil ribonucleotides. Sometimes the nucleic acid of a reporter has only uracil ribonucleotides. In some cases, the nucleic acid of a reporter comprises at least one adenine ribonucleotide. In some cases, the nucleic acid of a reporter comprises at least two adenine ribonucleotides. In some cases, the nucleic acid of a reporter has only adenine ribonucleotides. In some cases, the nucleic acid of a reporter comprises at least one cytosine ribonucleotide. In some cases, the nucleic acid of a reporter comprises at least two cytosine ribonucleotides. In some cases, the nucleic acid of a reporter comprises at least one guanine ribonucleotide. In some cases, the nucleic acid of a reporter comprises at least two guanine ribonucleotides. In some instances, a nucleic acid of a reporter comprises a single unmodified ribonucleotide. In some instances, a nucleic acid of a reporter comprises only unmodified ribonucleotides.

In some cases, the reporter nucleic acid is a single-stranded nucleic acid sequence comprising deoxyribonucleotides. The nucleic acid of a reporter may be a single-stranded nucleic acid sequence comprising at least one deoxyribonucleotide. In some cases, the nucleic acid of a reporter is a single-stranded nucleic acid comprising at least one deoxyribonucleotide residue at an internal position that functions as a cleavage site. In some cases, the nucleic acid of a reporter comprises at least 2, at least 3, at least 4, at least 5, at least 6, at least 7, at least 8, at least 9, or at least 10 deoxyribonucleotide residues at an internal position. In some cases, the nucleic acid of a reporter comprises from 2 to 10, from 3 to 9, from 4 to 8, or from 5 to 7 deoxyribonucleotide residues at an internal position. Sometimes the deoxyribonucleotide residues are continuous. Alternatively, the deoxyribonucleotide residues are interspersed in between non-deoxyribonucleotide residues. In some cases, the nucleic acid of a reporter has only deoxyribonucleotide residues. In some cases, the nucleic acid of a reporter comprises at least one deoxyribonucleotide residue and at least one non-deoxyribonucleotide residue.

In some cases, the nucleic acid of a reporter comprises at least one thymine deoxyribonucleotides. In some cases, the nucleic acid of a reporter comprises at least two thymine deoxyribonucleotides. Sometimes the nucleic acid of a reporter has only thymine deoxyribonucleotides. In some cases, the nucleic acid of a reporter comprises at least one adenine deoxyribonucleotides. In some cases, the nucleic acid of a reporter comprises at least two adenine deoxyribonucleotides. In some cases, the nucleic acid of a reporter has only adenine deoxyribonucleotides. In some cases, the nucleic acid of a reporter comprises at least one cytosine deoxyribonucleotides. In some cases, the nucleic acid of a reporter comprises at least two cytosine deoxyribonucleotides. In some cases, the nucleic acid of a reporter comprises at least one guanine deoxyribonucleotides. In some cases, the nucleic acid of a reporter comprises at least two guanine deoxyribonucleotides. In some instances, a nucleic acid of a reporter comprises a single unmodified deoxyribonucleotide. In some instances, a nucleic acid of a reporter comprises only unmodified deoxyribonucleotides.

In some cases, the reporter nucleic acid is a single-stranded nucleic acid sequence comprising ribonucleotides and deoxyribonucleotides. The nucleic acid of a reporter may be a single-stranded nucleic acid sequence comprising at least one ribonucleotide and at least one deoxyribonucleotide. In some cases, the nucleic acid of a reporter has only ribonucleotide residues and deoxyribonucleotide residues.

In some cases, the nucleic acid comprises nucleotides resistant to cleavage by the effector protein described herein. In some cases, the nucleic acid of a reporter comprises synthetic nucleotides.

In some cases, the nucleic acid of a reporter is 5 to 20, 5 to 15, 5 to 10, 7 to 20, 7 to 15, or 7 to 10 nucleotides in length. In some cases, the nucleic acid of a reporter is 3 to 20, 4 to 10, 5 to 10, or 5 to 8 nucleotides in length. In some cases, the nucleic acid of a reporter is 5 to 12 nucleotides in length. In some cases, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the reporter nucleic acid is 2, 3, 4, 5,6,7, 8,9, 10, 11, 12, 13, 14, 15, 16, 17, 18, 19, 20, 21, 22, 23, 24, 25, 26, 27, 28, 29, or 30 nucleotides in length.

In some cases, systems comprise a plurality of reporters. The plurality of reporters may comprise a plurality of signals. In some cases, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some cases, there are 2 to 50, 3 to 40, 4 to 30, 5 to 20, or 6 to 10 different reporters.

In some instances, systems comprise an effector protein and a reporter nucleic acid configured to undergo transcollateral cleavage by the effector protein. Transcollateral cleavage of the reporter may generate a signal from reporter or alter a signal from the reporter. In some cases, the signal is an optical signal, such as a fluorescence signal or absorbance band. Transcollateral cleavage of the reporter may alter the wavelength, intensity, or polarization of the optical signal. For example, the reporter may comprise a fluorophore and a quencher, such that transcollateral cleavage of the reporter separates the fluorophore and the quencher thereby increasing a fluorescence signal from the fluorophore. Herein, detection of reporter cleavage (directly or indirectly) to determine the presence of a target nucleic acid sequence may be referred to as “DETECTR”. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein may be inhibited. This is because the activated effector proteins collaterally cleaves any nucleic acids. If total nucleic acids are present in large amounts, they may outcompete reporters for the effector proteins. In some instances, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some instances, the sample comprises amplified target nucleic acid. In some instances, the sample comprises an unamplified target nucleic acid. In some instances, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some instances, systems comprise a reporter wherein the concentration of the reporter in a solution 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. In some instances, systems comprise a reporter wherein the concentration of the reporter in a solution is 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents/Components

In some embodiments, systems comprise a reagent or component for amplifying a nucleic acid. Systems may also comprise at least one amplification reagent or component for amplifying a target nucleic acid. Non-limiting examples of reagents for amplifying a nucleic acid include polymerases, primers, and nucleotides. In some instances, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some instances, nucleic acid amplification is isothermal nucleic acid amplification, providing for the use of the system or system in remote regions or low resource settings without specialized equipment for amplification. In some cases, amplification of the target nucleic acid increases the concentration of the target nucleic acid in the sample relative to the concentration of nucleic acids that do not correspond to the target nucleic acid.

An amplification reagent, in some instances, comprises a primer, an activator, a deoxynucleoside triphosphate (dNTP), a ribonucleoside triphosphate (rNTP), or combinations thereof. An amplification reagent may comprise a primer. An amplification reagent may comprise an activator. An amplification reagent may comprise a dNTP. An amplification reagent may comprise an rNTP.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some instances, systems comprise a PCR tube, a PCR well or a PCR plate. The wells of the PCR plate may be pre-aliquoted with the reagent for amplifying a nucleic acid, as well as an engineered guide nucleic acid, an effector protein, a multimeric complex, or any combination thereof. The wells of the PCR plate may be pre-aliquoted with an engineered guide nucleic acid targeting a target sequence, an effector protein capable of being activated when complexed with the engineered guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; an engineered guide nucleic acid targeting a target sequence; an effector protein capable of being activated when complexed with the engineered guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; an engineered guide nucleic acid targeting a target sequence; and an effector protein capable of being activated when complexed with the engineered guide nucleic acid and the target sequence. In some cases, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; an engineered guide nucleic acid targeting a target sequence; and an effector protein capable of being activated when complexed with the engineered guide nucleic acid and the target sequence. The wells of the PCR plate may be pre-aliquoted with the engineered guide nucleic acid targeting a target sequence, and an effector protein capable of being activated when complexed with the engineered guide nucleic acid and the target sequence. A user may thus add the biological sample of interest to a well of the pre-aliquoted PCR plate.

In some embodiments, systems comprise a support medium; a guide nucleic acid targeting a target sequence; and a programmable nuclease capable of being activated when complexed with the guide nucleic acid and the target sequence. In some cases, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for detecting and/or modifying a target nucleic acid comprises a PCR plate; a guide nucleic acid targeting a target sequence; and a programmable nuclease capable of being activated when complexed with the guide nucleic acid and the target sequence. The wells of the PCR plate may be pre-aliquoted with the guide nucleic acid targeting a target sequence, and a programmable nuclease capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20° C. to 80° C., for example around 20° C. to 45° C. or 45° C. to 80° C. In some cases, the nucleic acid amplification reaction is performed at a temperature no greater than 20° C., 25° C., 30° C., 35° C., 37° C., 40° C., 45° C., 50° C., 55° C., 60° C., 65° C., 70° C., 75° C., 80° C. or any value 20° C. to 80° C. In some cases, the nucleic acid amplification reaction is performed at a temperature of at least 20° C., 25° C., 30° C., 35° C., 37° C., 40° C., 45° C., 50° C., 55° C., 60° C., 65° C., 70° C., 75° C., or 80° C., or any value 20° C. to 80° C. In some cases, the nucleic acid amplification reaction is performed at a temperature of 20° C. to 45° C., 25° C. to 40° C., 30° C. to 40° C., or 35° C. to 40° C. In some cases, the nucleic acid amplification reaction is performed at a temperature of 45° C. to 80° C., 50° C. to 75° C., 55° C. to 70° C., or 60° C. to 65° C. In some cases, the nucleic acid amplification reaction is performed using thermocycling between any two or more temperatures described herein.

Often, systems comprise primers for amplifying a target nucleic acid to produce an amplification product comprising the target nucleic acid and a PAM. For instance, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence.

### Additional System Components

In some instances, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A system may include labels listing contents and/or instructions for use, or package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some instances, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### B. Certain Conditions of Systems

In some instances, systems and methods are employed under certain conditions that enhance an activity of the effector protein, a dimer thereof, or a multimeric complex thereof, relative to alternative conditions, as measured by a detectable signal released from cleavage of a reporter in the presence of the target nucleic acid. The detectable signal may be generated at about the rate of transcollateral cleavage of a reporter nucleic acid. In some instances, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some instances, the reporter is an RNA-FQ reporter. In some instances, the reporter is a DNA-FQ reporter.

In some instances, effector proteins, dimers, multimeric complexes, or combinations thereof recognize, bind, or are activated by, different target nucleic acids having different sequences, but are active toward the same reporter nucleic acid, allowing for facile multiplexing in a single assay having a single ssRNA-FQ reporter or a single ssDNA-FQ reporter.

In some instances, systems are employed under certain conditions that enhance transcollateral cleavage activity of the effector protein. In some instances, under certain conditions, transcolatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some instances, systems and methods are employed under certain conditions that enhance cis-cleavage activity of the effector protein.

In some instances, under certain conditions, transcollateral cleavage occurs at an enzyme turnover rate (Kcat) of at least 0.5 per second, at least 0.6 per second, at least 0.7 per second, at least 0.8 per second, at least 0.9 per second, at least 1 per second, at least 2 per second, at least 3 per second, at least 4 per second, at least 5 per second, at least 6 per second, at least 7 per second, at least 8 per second, at least 9 per second, at least 10 per second, at least 20 per second, at least 30 per second, at least 40 per second, at least 50 per second, at least 60 per second, at least 70 per second, at least 80 per second, at least 90 per second, at least 100 per second, at least 150 per second, at least 200 per second, at least 250 per second, at least 300 per second, at least 350 per second, at least 400 per second, at least 450 per second, at least 500 per second, at least 550 per second, at least 600 per second, at least 650 per second, at least 700 per second, at least 750 per second, at least 800 per second, at least 850 per second, at least 900 per second, at least 950 per second, at least 1000 per second.

In some instances, under certain conditions, transcollateral cleavage occurs with a Michaelis-Menten constant (K_{M}) of at least 500 nM, 750 nM, 1000 nM, 1250 nM, 1500 nM, 2000 nM, 3000 nM, 4000 nM, 5000 nM, 6000 nM, 7000 nM, 8000 nM, 9000 nM, 10000 nM, 11000 nM, 12000 nM, 13000 nM, 14000 nM, 15000 nM, 16000 nM, 17000 nM, 18000 nM, 19000 nM, 20000 nM, 25000 nM, 30000 nM. In some instances, under certain conditions, transcollateral cleavage occurs with a Michaelis-Menten constant (K_{M}) of at most 500 nM, 750 nM, 1000 nM, 1250 nM, 1500 nM, 2000 nM, 3000 nM, 4000 nM, 5000 nM, 6000 nM, 7000 nM, 8000 nM, 9000 nM, 10000 nM, 11000 nM, 12000 nM, 13000 nM, 14000 nM, 15000 nM, 16000 nM, 17000 nM, 18000 nM, 19000 nM, 20000 nM, 25000 nM, 30000 nM.

In some instances, under certain conditions, transcollateral cleavage occurs with a kinetic efficiency (Kcat/Km) of at least 1×10⁵ M⁻¹s⁻¹, at least 2×10⁵ M⁻¹s⁻¹, at least 3×10⁵ M⁻¹s⁻¹, at least 4×10⁵ M⁻¹s⁻¹, at least 5×10⁵ M⁻¹s⁻¹, at least 6×10⁵ M⁻¹s⁻¹, at least 7×10⁵ M⁻¹s⁻¹, at least 8×10⁵ M⁻¹s⁻¹, at least 9×10¹ M⁻¹s⁻¹, at least 1×10⁶ M⁻¹s⁻¹, at least 2×10⁶ M⁻¹s⁻¹, at least 3×10⁶ M⁻¹s⁻¹, at least 4×10⁶ M⁻¹ s⁻¹ at least 5×10⁶ M⁻¹s⁻¹, at least 6×10⁶ M⁻¹s⁻¹ at least 7×10⁶ M⁻¹s⁻¹, at least 8×10⁶ M⁻¹s⁻¹, at least 9×10⁶ M⁻¹s⁻¹, at least 1×10⁷ M⁻¹s⁻¹, at least 2×10⁷ M⁻¹s⁻¹, at least 3×10⁷ M⁻¹s⁻¹ at least 4×10⁷ M⁻¹s⁻¹ at least 5×10⁷ M⁻¹s⁻¹, at least 6×10⁷ M⁻¹s⁻¹, at least 7×10⁷ M⁻¹s⁻¹, at least 8×10⁷ M⁻¹s⁻¹, at least 9×10⁷ M⁻¹s⁻¹, or at least 1×10⁸ M⁻¹s⁻¹.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some instances, the salt is NaCl. In some instances, the salt is KNO₃. In some instances, the salt concentration is less than 150 mM, less than 125 mM, less than 100 mM, less than 75 mM, less than 50 mM, or less than 25 mM.

Certain conditions that may enhance the activity of an effector protein includes the pH of a solution in which the activity. For example, increasing pH may enhance transcollateral activity. For example, the rate of transcollateral activity may increase with increase in pH up to pH 9. In some instances, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some instances, the pH is 7 to 7.5, 7.5 to 8, 8 to 8.5, 8.5 to 9, or 7 to 8.5. In some cases, the pH is less than 7. In some cases, the pH is greater than 7.

Certain conditions that may enhance the activity of an effector protein includes the temperature at which the activity is performed. In some instances, the temperature is about 25° C. to about 80° C. In some instances, the temperature is about 20° C. to about 40° C., about 30° C. to about 50° C., about 40° C. to about 60° C., about 50° C. to about 70° C., or about 60° C. to about 80° C. In some instances, the temperature is about 25° C., about 30° C., about 35° C., about 40° C., about 45° C., about 50° C., about 55° C., about 60° C., about 65° C., about 70° C., about 75° C., or about 80° C.

In some instances, a final concentration of an effector protein or multimeric complex thereof in a buffer of a system is 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM. The final concentration of the sgRNA complementary to the target nucleic acid may be 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM. The concentration of the ssDNA-FQ reporter may be 1 pM to 1 nM, 1 pM to 10 pM, 10 pM to 100 pM, 100 pM to 1 nM, 1 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1000 nM.

In some instances, systems comprise an excess volume of solution comprising the engineered guide nucleic acid, the effector protein, and the reporter, which contacts a smaller volume comprising a sample with a target nucleic acid. The smaller volume comprising the sample may be unlysed sample, lysed sample, or lysed sample which has undergone any combination of reverse transcription, amplification, and in vitro transcription. The presence of various reagents, (such as buffer, magnesium sulfate, salts, the pH, a reducing agent, primers, dNTPs, NTPs, cellular lysates, non-target nucleic acids, primers, or other components), in a crude, non-lysed sample, a lysed sample, or a lysed and amplified sample, may inhibit the ability of the effector protein to become activated or to find and cleave the nucleic acid of the reporter. This may be due to nucleic acids that are not the reporter outcompeting the nucleic acid of the reporter, for the effector protein. Alternatively, various reagents in the sample may simply inhibit the activity of the effector protein. Thus, the compositions and methods provided herein for contacting an excess volume comprising the engineered guide nucleic acid, the effector protein, and the reporter to a smaller volume comprising the sample with the target nucleic acid of interest provides for superior detection of the target nucleic acid by ensuring that the effector protein is able to find and cleaves the nucleic acid of the reporter. In some embodiments, the volume comprising the engineered guide nucleic acid, the effector protein, and the reporter (may be referred to as “a second volume”) is 4-fold greater than a volume comprising the sample (may be referred to as “a first volume”). In some embodiments, the volume comprising the engineered guide nucleic acid, the effector protein, and the reporter (may be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold greater than a volume comprising the sample (may be referred to as “a first volume”). In some embodiments, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, 0.5 μL to 5 μL μL, 5 μL to 10 μL, 10 μL to 15 μL, 15 μL to 20 μL, 20 μL to 25 μL, 25 μL to 30 μL, 30 μL to 35 μL, 35 μL to 40 μL, 40 μL to 45 μL, 45 μL to 50 μL, 10 μL to 20 μL, 5 μL to 20 μL, 1 μL to 40 μL, 2 μL to 10 μL, or 1 μL to 10 μL. In some embodiments, the volume comprising the effector protein, the engineered guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, 10 μL to 15 μL μL, 15 μL to 20 μL, 20 μL to 25 μL, 25 μL to 30 μL, 30 μL to 35 μL, 35 μL to 40 μL, 40 μL to 45 μL, 45 μL to 50 μL, 50 μL to 55 μL, 55 μL to 60 μL, 60 μL to 65 μL, 65 μL to 70 μL, 70 μL to 75 μL, 75 μL to 80 μL, 80 μL to 85 μL, 85 μL to 90 μL, 90 μL to 95 μL, 95 μL to 100 μL, 100 μL to 150 μL, 150 μL to 200 μL, 200 μL to 250 μL, 250 μL to 300 μL, 300 μL to 350 μL, 350 μL to 400 μL, 400 μL to 450 μL, 450 μL to 500 μL, 10 μL to 20 μL, 10 μL to 30 μL, 25 μL to 35 μL, 10 μL to 40 μL, 20 μL to 50 μL, 18 μL to 28 μL, or 17 μL to 22 μL.

In some instances, systems comprise an effector protein that nicks a target nucleic acid, thereby producing a nicked product. In some instances, systems cleave a target nucleic acid, thereby producing a linearized product. In some cases, systems produce at least 50%, at least 55%, at least 60%, at least 65%, at least 70%, at least 75%, at least 80%, at least 85%, at least 90 or at least 95% of a maximum amount of nicked product within 1 minute, where the maximum amount of nicked product is the maximum amount detected within a 60 minute period from when the target nucleic acid is mixed with the effector protein or the multimeric complex thereof. In some cases, systems produce at least 50%, at least 55%, at least 60%, at least 65%, at least 70%, at least 75%, at least 80%, at least 85%, at least 90 or at least 95% of a maximum amount of linearized product within 1 minute, where the maximum amount of linearized product is the maximum amount detected within a 60 minute period from when the target nucleic acid is mixed with the effector protein. In some cases, at least 80% of the maximum amount of linearized product is produced within 1 minute. In some cases, at least 90% of the maximum amount of linearized product is produced within 1 minute.

### C. Certain Systems

In some cases, systems comprise a DNA Endonuclease Targeted CRISPR TransReporter (DETECTR) assay, and a programmable nuclease disclosed herein, a dimer thereof, or a multimeric complex thereof. The principles of the DETECTR assay are described in Chen et al. (Science 2018 Apr. 27; 360(6387): 436-439) and may be modified to facilitate the use of the programmable nucleases described herein. A DETECTR assay may utilize the trans-cleavage abilities of programmable nucleases to achieve fast and high-fidelity detection of a target nucleic acid in a sample. For example, following target RNA extraction from a biological sample, crRNA comprising a portion that is complementary to the target RNA of interest may bind to the target RNA sequence, initiating indiscriminate ssRNase activity by the programmable nuclease. Upon hybridization with the target RNA, the trans-cleavage activity of the programmable nuclease is activated, which may then cleave an ssDNA fluorescence-quenching (FQ) reporter molecule (e.g., an RNA molecule comprising a fluorophore and a fluorescence quenching moiety that may separate upon cleavage of the RNA molecule). Cleavage of the reporter molecule may provide a fluorescent readout indicating the presence of the target RNA in the sample. In some embodiments, the programmable nucleases disclosed herein may be combined, or multiplexed, with other programmable nucleases in a DETECTR assay.

An example of a system for a DETECTR assay comprises final concentrations of 100 nM Type V CRISPR/Cas protein, 125 nM sgRNA, and 50 nM ssRNA-FQ reporter in a total reaction volume of 20 μL. The Type V CRISPR/Cas protein or variant thereof may form a homodimeric complex configured to bind a single guide nucleic acid and a single target nucleic acid molecule. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 nm). The fluorescence wavelength detected may vary depending on the reporter molecule.

Another example of a system for a DETECTR assay comprises final concentrations of 40 nM Type V CRISPR/Cas protein, 40 nM gRNA, and 50 nM ssDNA-FQ reporter in a total reaction volume of 20 μL. The Type V CRISPR/Cas protein or variant thereof may bind a single guide nucleic acid and a single target nucleic acid molecule. Reactions are incubated in a fluorescence plate reader (Tecan Infinite Pro 200 M Plex) for 30 minutes at 37° C. with fluorescence measurements taken every 30 seconds (e.g., λex: 485 nm; λem: 535 nm). The fluorescence wavelength detected may vary depending on the reporter molecule.

In some instances, a DETECTR assay is used to detect an amplified target nucleic acid, wherein the amplified target nucleic acid is present in an amount relative to an amount of a programmable nuclease. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present at 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the programmable nuclease. In some embodiments, the programmable nuclease is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the target nucleic acid. In some embodiments, the target nucleic acid is not present in the sample.

In some instances, a DETECTR assay is used to detect an amplified target nucleic acid, wherein the amplified target nucleic acid is present in an amount relative to an amount of an engineered guide nucleic acid. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the engineered guide nucleic acid. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the engineered guide nucleic acid. In some embodiments, the amplified target nucleic acid is present in 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the engineered guide nucleic acid. In some embodiments, the engineered guide nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the engineered guide nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the engineered guide nucleic acid is present in 1-fold to 2-fold, 1-fold to 3-fold, 1-fold to 4-fold, 1-fold to 5-fold, 1-fold to 10-fold, 1-fold to 25-fold, 1-fold to 50-fold, 1-fold to 100-fold, 1-fold to 500-fold, 1-fold to 1000-fold, 1-fold to 10,000-fold, 1-fold to 100,000-fold, 5-fold to 10-fold, 5-fold to 25-fold, 5-fold to 50-fold, 5-fold to 100-fold, 5-fold to 500-fold, 5-fold to 1000-fold, 5-fold to 10,000-fold, 5-fold to 100,000-fold, 10-fold to 25-fold, 10-fold to 50-fold, 10-fold to 100-fold, 10-fold to 500-fold, 10-fold to 1000-fold, 10-fold to 10,000-fold, 10-fold to 100,000-fold, 100-fold to 500-fold, 100-fold to 1000-fold, 100-fold to 10,000-fold, 100-fold to 100,000-fold, 1000-fold to 10,000-fold, 1000-fold to 100,000-fold, or 10,000-fold to 100,000-fold molar excess relative to the amount of the target nucleic acid. In some embodiments, the target nucleic acid is not present in the sample.

In some cases, systems comprise a specific high-sensitivity enzymatic reporter unlocking (SHERLOCK) assay, and a programmable nuclease disclosed herein, a dimer thereof, or a multimeric complex thereof. The SHERLOCK assay is described in Kellner et al. (Nat Protoc. 2019 October; 14(10):2986-3012) and may be modified to facilitate the use of the programmable nucleases described herein.

In some instances, systems for detecting a target nucleic acid comprise a support medium; an engineered guide nucleic acid targeting a target sequence; a programmable nuclease capable of being activated when complexed with the engineered guide nucleic acid and the target sequence; and a reporter nucleic acid comprising a detection moiety, wherein the reporter nucleic acid is capable of being cleaved by the activated nuclease, thereby generating a first detectable signal.

In some instances, systems for detecting a target nucleic acid are configured to perform one or more steps of the DETECTR assay in a volume or on the support medium. In some instances, one or more steps of the DETECTR assay are performed in the same volume or at the same location on the support medium. For example, target nucleic acid amplification can occur in a separate volume before the RNP is contacted to the amplified target nucleic acids. In another example, target nucleic acid amplification can occur in the same volume in which the target nucleic acids complex with the RNP (e.g., amplification can occur in a sample well or tube before the RNP is added and/or amplification and RNP complexing can occur in the sample well or tube simultaneously). Detection of the detectable signal indicative of transcollateral cleavage of the reporter nucleic acid can occur in the same volume or location on the support medium (e.g., sample well or tube after or simultaneously with transcleavage) or in a different volume or location on the support medium (e.g., at a detection location on a lateral flow assay strip). In some instances, all steps of the DETECTR assay can be performed in the same volume or at the same location on the support medium. For example, target nucleic acid amplification, complexing of the RNP with the target nucleic acid, transcollateral cleavage of the reporter nucleic acid, and generation of the detectable signal can occur in the same volume (e.g., sample well or tube). Alternatively, or in combination, target nucleic acid amplification, complexing of the RNP with the target nucleic acid, transcollateral cleavage of the reporter nucleic acid, and generation of the detectable signal can occur at the same location on the support medium (e.g., on a bead in a well or flow channel).

### V. Methods of Nucleic Acid Detection

Provided herein are methods of detecting target nucleic acids. Methods may comprise detecting target nucleic acids with compositions or systems described herein. Methods may comprise detecting the presence or absence of target nucleic acids with compositions or systems described herein. Methods may comprise detecting a target nucleic acid in a sample, e.g., a cell lysate, a biological fluid, or environmental sample, or an amplified portion thereof. Methods may comprise detecting a target nucleic acid in a cell. In some instances, methods of detecting a target nucleic acid in a sample or cell comprises a) contacting the sample or cell or a portion thereof (e.g., a lysate or amplification product) with i) an effector protein, ii) an engineered guide nucleic acid, wherein at least a portion of the engineered guide nucleic acid is complementary to at least a portion of the target nucleic acid, and iii) a reporter nucleic acid that is cleaved in the presence of the effector protein, the engineered guide nucleic acid, and the target nucleic acid, and b) detecting a signal indicative of (e.g., produced by) cleavage of the reporter nucleic acid, thereby detecting the target nucleic acid in the sample. In some instances, methods result in transcollateral cleavage of the reporter nucleic acid. In some instances, methods result in cis cleavage of the reporter nucleic acid. In some instances, the effector protein comprises an amino acid sequence that is at least is at least 70%, at least 75%, at least 80%, at least 85%, at least 90%, at least 92%, at least 95%, at least 97%, at least 98%, at least 99%, or 100% identical to any one of SEQ ID NOs: 1-4. In some instances, the amino acid sequence of the effector protein is at least is at least 70%, at least 75%, at least 80%, at least 85%, at least 90%, at least 92%, at least 95%, at least 97%, at least 98%, at least 99%, or 100% identical to any one of SEQ ID NOs: 1-4. In some instances, the engineered guide nucleic acid comprises a nucleobase sequence that is at least 75%, at least 80%, at least 85%, at least 90%, at least 95%, at least 98%, at least 99% or 100% identical to any one of SEQ ID NOs: 19-21.

In some instances, the methods cleave at least about 50%, 60%, 70%, 80%, 100%, 2-fold, 3-fold, 4-fold, 5-fold, 10-fold, at least 15-fold, at least 20-fold, at least 25-fold, at least 30-fold, at least 35-fold, at least 40-fold, at least 45-fold, at least 50-fold or more of that at 40° C. of the target nucleic acids in a sample, wherein the temperature of the sample is at least about 37° C., 40° C., 45° C., 50° C., 55° C., 60° C., 65° C., 70° C., 75° C., 80° C., 85° C., or 90° C.

Methods may comprise contacting the sample to a complex comprising an engineered guide nucleic acid comprising a segment that is reverse complementary to a segment of the target nucleic acid and an effector protein that exhibits sequence independent cleavage upon forming a complex comprising the segment of the engineered guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample or cell with an effector protein and an engineered guide nucleic acid at a temperature of at least about 25° C., at least about 30° C., at least about 35° C., at least about 40° C., at least about 50° C., or at least about 65° C. In some instances, the temperature is not greater than 80° C. In some instances, the temperature is about 25° C., about 30° C., about 35° C., about 40° C., about 45° C., about 50° C., about 55° C., about 60° C., about 65° C., or about 70° C. In some instances, the temperature is about 25° C. to about 45° C., about 35° C. to about 55° C., or about 55° C. to about 65° C.

Methods may comprise cleaving a strand of a single-stranded target nucleic acid with an effector protein or a multimeric complex thereof, as assessed with an in vitro cis-cleavage assay. An example of such an assay may follow a procedure comprising: (i) providing equimolar (e.g., 500 nM) amounts of an effector protein comprising at least 70% sequence identity to any one of SEQ ID NOs: 1-4 and an engineered guide nucleic acid at 40 to 45° C. for 5 minutes in pH 7.5 Tris-HCl buffer, 40 mM NaCl, 2 mM Ca(NO₃)₂, 1 mM BME, thereby forming a ribonucleoprotein complex comprising a dimer of the effector protein and the engineered guide nucleic acid; (ii) adding linear dsDNA comprising a nucleic acid sequence targeted by the engineered guide nucleic acid and adjacent to a PAM comprising the sequence 5′-TTTA-3′; (iii) incubating the mixture at 45° C. for 20 minutes, thereby enabling cleavage of the plasmid; (iv) quenching the reaction with EDTA and a protease; and (v) analyzing the reaction products (e.g., viewing the cleaved and uncleaved linear dsDNA with gel electrophoresis).

In some cases, there is a threshold of detection for methods of detecting target nucleic acids. In some instances, methods are not capable of detecting target nucleic acids that are present in a sample or solution at a concentration less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases the threshold of detection is in a range of from 2 aM to 100 pM, from 20 aM to 50 pM, from 50 aM to 20 pM, from 200 aM to 5 pM, or from 500 aM to 2 pM. In some cases, the threshold of detection is less than 250 pM, less than 25 pM, less than 2.5 pM, or less than 250 fM of the target nucleic acid at a temperature within a range of from about 45° C. to about 80° C.

In some cases, the minimum concentration at which a target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a target nucleic acid is detected in a sample is in a range of from 2 aM to 100 pM, from 20 aM to 50 pM, from 50 pM to 20 pM, from 200 aM to 5 pM, or from 500 aM to 2 pM. In some cases, the minimum concentration at which a single stranded target nucleic acid can be detected in a sample is in a range of from 1 aM to 100 pM. In some cases, the minimum concentration at which a target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some instances, the target nucleic acid is present in a sample at a concentration of about 1 pM, about 10 pM, about 100 pM, about 200 pM, about 300 pM, about 400 pM, about 500 pM, about 600 pM, about 700 pM, about 800 pM, about 900 pM, about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 1 μM to 100 μM, 100 μM to 250 μM, 250 μM to 500 μM, 500 μM to 1 nM, 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 1 μM to 1 nM, from 100 μM to 500 μM, from 500 μM to 100 nM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 1 μM to 1 nM, from 200 μM to 20 nM, from 20 nM to 50 μM, from 50 nM to 20 μM, or from 200 nM to 5 μM.

In some cases, methods detect a target nucleic acid in less than 60 minutes. In some cases, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some cases, methods of detecting are performed in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, or less than about 5 minutes. In some cases, methods of detecting are performed in about 5 minutes to about 120 minutes, about 5 minutes to about 100 minutes, about 10 minutes to about 90 minutes, about 15 minutes to about 45 minutes, about 20 minutes to about 35 minutes.

In some cases, methods of detecting are performed in less than about 10 hours, less than about 9 hours, less than about 8 hours, less than about 7 hours, less than about 6 hours, less than about 5 hours, less than about 4 hours, less than about 3 hours, less than about 2 hours, less than about 1 hour, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 9 minutes, less than about 8 minutes, less than about 7 minutes, less than about 6 minutes, or less than about 5 minutes. In some cases, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

Methods may comprise detecting a detectable signal within 5 minutes of contacting the sample and/or the target nucleic acid with the engineered guide nucleic acid and/or the effector protein. In some cases, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

Provided herein are methods of detecting a target nucleic acid, the method comprising: a. contacting the sample with: i. an effector protein, wherein the effector protein provides transcollateral cleavage activity on a target nucleic acid at a temperature within a range of about 45° C. to 80° C., ii. an engineered guide nucleic acid, and iii. a detection reagent that is cleaved in the presence of the effector protein, the engineered guide nucleic acid, and the target nucleic acid; and b. detecting a signal indicative of cleavage of the detection reagent, thereby detecting the target nucleic acid. In some cases, the effector protein comprises an amino acid sequence that is at least 75%, at least 80%, at least 85%, at least 90%, at least 95%, at least 98%, at least 99%, or 100% identical to SEQ ID NO: 2. In some cases, the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T.

Provided herein are uses of a composition comprising: a. heating a sample to a temperature within a range of about 45° C. to 80° C.; and b. contacting the heated sample with a composition comprising: i. an effector protein, wherein the effector protein provides transcollateral cleavage activity on a target nucleic acid at a temperature within a range of about 45° C. to 80° C., ii. an engineered guide nucleic acid, and iii. a detection reagent that is cleaved in the presence of the effector protein, the engineered guide nucleic acid, and a target nucleic acid, wherein the contacting happens at a temperature above 45° C. In some cases, the effector protein comprises an amino acid sequence that is at least 75%, at least 80%, at least 85%, at least 90%, at least 95%, at least 98%, at least 99%, or 100% identical to SEQ ID NO: 2. In some cases, the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T.

In some cases, a method of detecting a target nucleic acid includes detecting transcollateral cleavage activity with a lateral flow device. In some cases, detecting transcollateral cleavage activity with a lateral flow device comprises applying the supernatant from a detection reaction to a lateral flow assay strip.

In some cases, a method of detecting a target nucleic acid is performed using a lateral flow device.

A lateral flow device comprising effector proteins, compositions, complexes or systems disclosed herein is provided.

In some cases, a lateral flow device comprises a composition or complex disclosed herein and a detection reagent.

In some cases, a lateral flow device comprises an effector protein and an engineered guide nucleic acid disclosed herein and a detection reagent.

### A. Amplifying Target Nucleic Acids

Methods may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid. Amplifying may also comprise an amplification reagent. An amplification reagent, in some instances, comprises a primer, an activator, a deoxynucleoside triphosphate (dNTP), a ribonucleoside triphosphate (rNTP), or combinations thereof. An amplification reagent may comprise a primer. An amplification reagent may comprise an activator. An amplification reagent may comprise a dNTP. An amplification reagent may comprise an rNTP.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA). An amplification may also comprise isothermal amplification.

In some embodiments, amplification of the target nucleic acid comprises modifying the sequence of the target nucleic acid. For example, amplification may be used to insert a PAM sequence into an amplicon generated from a target nucleic acid that lacks a PAM sequence. In some cases, amplification may be used to increase the homogeneity of a target nucleic acid in a sample. For example, amplification may be used to remove a nucleic acid variation that is not of interest in the target nucleic acid sequence.

Amplifying may take 1, 2, 3, 4, 5, 6, 7, 8, 9, 10, 11, 12, 13, 14, 15, 16, 17, 18, 19, 20, 25, 30, 40, 50, or 60 minutes.

In some instances, methods of detecting comprise amplifying a target nucleic acid. In some instances, all the steps of the method are performed at the same temperature that amplification occurs. Amplifying may be performed at a temperature of about 20° C. to about 45° C. Amplifying may be performed at a temperature of less than about 20° C., less than about 25° C., less than about 30° C., 35° C., less than about 37° C., less than about 40° C., or less than about 45° C. Amplifying may be performed at a temperature of at least about 20° C., at least about 25° C., at least about 30° C., at least about 35° C., at least about 37° C., at least about 40° C., at least about 45° C., at least about 50° C., at least about 55° C., at least about 60° C., at least about 65° C. Amplifying may be performed at a temperature of about 20° C., about 25° C., about 30° C., about 35° C., about 37° C., about 40° C., about 45° C., about 50° C., about 55° C., about 60° C., or about 65° C.

In some cases, the method may also comprise reverse transcribing the target nucleic acid and/or amplifying the target nucleic acid before contacting the sample with the effector protein, the engineered guide nucleic acid, and the detection reagent. In some cases, the method may also comprise reverse transcribing the target nucleic acid and/or amplifying the target nucleic acid after contacting the sample with the effector protein, the engineered guide nucleic acid, and the detection reagent. In some cases, the contacting and the reverse transcribing are carried out at the same temperature. In some cases, the detecting and the reverse transcribing are carried out at the same temperature. In some cases, the contacting, the detecting, and the reverse transcribing are carried out at the same temperature. In some cases, the contacting and the amplifying are carried out at the same temperature. In some cases, the detecting and the amplifying are carried out at the same temperature. In some cases, the contacting, the detecting, and the amplifying are carried out at the same temperature. In some cases, the contacting, the detecting, the reverse transcribing, and the amplifying are carried out at the same temperature. The temperature may be about 25° C., about 30° C., about 35° C., about 37° C., about 40° C., about 45° C., about 50° C., about 55° C., about 60° C., about 65° C., 70° C., 75° C., or about 80° C. In some instances, the temperature may be from about 25° C. to about 80° C., from about 30° C. to about 80° C., from about 35° C. to about 80° C., from about 37° C. to about 80° C., from about 40° C. to about 80° C., from about 45° C. to about 80° C., from about 50° C. to about 80° C., from about 55° C. to about 80° C., from about 60° C. to about 80° C., from about 65° C. to about 80° C., from about 70° C. to about 80° C., from about 75° C. to about 80° C., from about 25° C. to about 30° C., from about 25° C. to about 35° C., from about 25° C. to about 37° C., from about 25° C. to about 40° C., from about 25° C. to about 45° C., from about 25° C. to about 50° C., from about 25° C. to about 55° C., from about 25° C. to about 60° C., from about 25° C. to about 65° C., from about 25° C. to about 70° C., or from about 25° C. to about 75° C.

In some cases, the contacting and the reverse transcribing are carried out in a single reaction chamber. In some cases, the detecting and the reverse transcribing are carried out in a single reaction chamber. In some cases, the contacting, the detecting, and the reverse transcribing are carried out in a single reaction chamber. In some cases, the contacting and the amplifying are carried out in a single reaction chamber. In some cases, the detecting and the amplifying are carried out in a single reaction chamber. In some cases, the contacting, the detecting, and the amplifying are carried out in a single reaction chamber. In some cases, the contacting, the detecting, the reverse transcribing, and the amplifying are carried out in a single reaction chamber.

In some cases, the contacting and the reverse transcribing are carried out simultaneously in a single reaction chamber. In some cases, the detecting and the reverse transcribing are carried out simultaneously in a single reaction chamber. In some cases, the contacting, the detecting, and the reverse transcribing are carried out in a single reaction chamber. In some cases, the contacting and the amplifying are carried out simultaneously in a single reaction chamber. In some cases, the detecting and the amplifying are carried out simultaneously in a single reaction chamber. In some cases, the contacting, the detecting, and the amplifying are carried out simultaneously in a single reaction chamber. In some cases, the contacting, the detecting, the reverse transcribing, and the amplifying are carried out simultaneously in a single reaction chamber.

In some instances, the effector protein, the engineered guide nucleic acid, and the detection reagent of the method are formulated in a solution. The solution may comprise the solutions or system solutions described thereof.

### B. Certain Methods of Detection

An illustrative method for detecting a target nucleic acid molecule in a sample comprises contacting the sample comprising the target nucleic acid molecule with (i) an effector protein comprising at least 75% sequence identity to a sequence selected from the group consisting of SEQ ID NOs: 1-4; (ii) an engineered guide nucleic acid comprising a region that binds to the effector protein and an additional region that binds to the target nucleic acid; and (iii) a labeled, single stranded RNA reporter; cleaving the labeled single stranded RNA reporter by the effector protein to release a detectable label; and detecting the target nucleic acid by measuring a signal from the detectable label. Detecting a target nucleic acid molecule may also comprise any detection reagents or methods described thereof.

A further illustrative method for detecting a target nucleic acid molecule in a sample comprises contacting the sample comprising the target nucleic acid molecule with (i) a dimeric protein complex comprising an effector protein comprising at least 75% sequence identity to a sequence selected from the group consisting of SEQ ID NOs: 1-4; (ii) an engineered guide nucleic acid comprising a first region that binds to the target nucleic acid; (iii) a nucleic acid comprising a first region that binds to the effector protein and an additional region that hybridizes to second region of the engineered guide nucleic acid; and (iv) a labeled, single stranded RNA reporter; cleaving the labeled single stranded RNA reporter by the effector protein to release a detectable label; and detecting the target nucleic acid by measuring a signal from the detectable label.

### VI. Multiplexing

The systems, devices, and methods described herein can be multiplexed in a number of ways. Multiplexing may include assaying for two or more target nucleic acids in a sample. Multiplexing can be spatial multiplexing wherein multiple different target nucleic acids are detected from the same sample at the same time, but the reactions are spatially separated. Often, the multiple target nucleic acids are detected using the same effector protein, but different guide nucleic acids. The multiple target nucleic acids sometimes are detected using the different effector proteins. Sometimes, multiplexing can be single reaction multiplexing wherein multiple different target acids are detected in a single reaction volume. Often, at least two different effector proteins are used in single reaction multiplexing. For example, multiplexing can be enabled by immobilization of multiple categories of reporters within a device, to enable detection of multiple target nucleic acids. Multiplexing allows for detection of multiple target nucleic acids in one kit or system. In some cases, the multiple target nucleic acids comprise different target nucleic acids to a virus. In some cases, the multiple target nucleic acids comprise different target nucleic acids associated with at least a first disease and a second disease. Multiplexing for one disease can increase at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. For example, multiplexing methods may comprise a single assay for a microorganism species using a first effector protein and an antibiotic resistance pattern in a microorganism using a second effector protein. Sometimes, multiplexing allows for discrimination between multiple target nucleic acids of different influenza strains, for example, influenza A and influenza B. Often, multiplexing allows for discrimination between multiple target nucleic acids, such as target nucleic acids that comprise different genotypes, for example, for a wild-type genotype and for a mutant (e.g., SNP) genotype. Multiplexing for multiple viral infections can provide the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel comprises assaying for a plurality of unique target nucleic acids in a plurality of aliquots from a sample, assaying for a control nucleic acid control in another aliquot of the sample, and quantifying a plurality of signals of the plurality of unique target nucleic acids by measuring signals produced by cleavage of reporters compared to the signal produced in the second aliquot. Often the plurality of unique target nucleic acids are from a plurality of viruses in the sample. Sometimes the quantification of a signal of the plurality correlates with a concentration of a unique target nucleic acid of the plurality for the unique target nucleic acid of the plurality that produced the signal of the plurality. The disease panel can be for any disease.

In some cases, the combination of a guide nucleic acid, an effector protein, and a single stranded reporter configured to detect one target nucleic acid is provided in its own reagent chamber or its own support medium. In this case, multiple reagent chambers or support mediums are provided, where each reagent chamber is designed to detect one target nucleic acid. In some cases, multiple different target nucleic acids may be detected in the same chamber or support medium.

In some instances, the multiplexed devices and methods detect at least 2 different target nucleic acids in a single reaction. In some instances, the multiplexed devices and methods detect at least 3 different target nucleic acids in a single reaction. In some instances, the multiplexed devices and methods detect at least 4 different target nucleic acids in a single reaction. In some instances, the multiplexed devices and methods detect at least 5 different target nucleic acids in a single reaction. In some cases, the multiplexed devices and methods detect at least 6, 7, 8, 9, or 10 different target nucleic acids in a single reaction.

### VII. DETECTR Immobilization

One or more components or reagents of an effector protein-based detection reaction may be suspended in solution or immobilized on a surface. Effector proteins, guide nucleic acids, and/or reporters may be suspended in solution or immobilized on a surface. For example, the reporter, effector protein, and/or guide nucleic acid can be immobilized on the surface of a chamber in a device as disclosed herein. In some cases, the reporter, effector protein, and/or guide nucleic acid can be immobilized on beads, such as magnetic beads, in a chamber of a device as disclosed herein where they are held in position by a magnet placed below the chamber. An immobilized effector protein can be capable of being activated and cleaving a free-floating or immobilized reporter. An immobilized guide nucleic acid can be capable of binding a target nucleic acid and activating an effector protein complexed thereto. An immobilized reporter can be capable of being cleaved by the activated effector protein, thereby generating a detectable signal.

Described herein are various methods to immobilize effector protein-based diagnostic reaction components to the surface of a reaction chamber or other surface (e.g., a surface of a bead). Any of the devices described herein may comprise one or more immobilized detection reagent components (e.g., effector protein, guide nucleic acid, and/or reporter). In certain instances, methods include immobilization of effector proteins (e.g., Cas proteins or Cas enzymes), reporters, and/or guide nucleic acids (e.g., gRNAs). In some embodiments, various effector protein-based diagnostic reaction components are modified with biotin. In some embodiments, these biotinylated effector protein-based diagnostic reaction components are immobilized on surfaces coated with streptavidin. In some embodiments, the biotin-streptavidin chemistries are used for immobilization of effector protein-based reaction components. In some embodiments, NHS-Amine chemistry is used for immobilization of effector protein-based reaction components. In some embodiments, amino modifications are used for immobilization of effector protein-based reaction components.

In some embodiments, the effector protein, guide nucleic acid, or the reporter are immobilized to a device surface by a linkage or linker. In some embodiments, the linkage comprises a covalent bond, a non-covalent bond, an electrostatic bond, a bond between streptavidin and biotin, an amide bond or any combination thereof. In some embodiments, the linkage comprises non-specific absorption. In some embodiments, the effector protein is immobilized to the device surface by the linkage, wherein the linkage is between the effector protein and the surface. In some embodiments, the reporter is immobilized to the device surface by the linkage, wherein the linkage is between the reporter and the surface. In some embodiments, the guide nucleic acid is immobilized to the surface by the linkage, wherein the linkage is between the 5′ end of the guide nucleic acid and the surface. In some embodiments, the guide nucleic acid is immobilized to the surface by the linkage, wherein the linkage is between the 3′ end of the guide nucleic acid and the surface.

In some embodiments, the effector protein, guide nucleic acid, or the reporter are immobilized to or within a polymer matrix. The polymer matrix may comprise a hydrogel. Co-polymerization of the effector protein, guide nucleic acid, or the reporter into the polymer matrix may result in a higher density of reporter/unit volume or reporter/unit area than other immobilization methods utilizing surface immobilization (e.g., onto beads, after matrix polymerization, etc.). Co-polymerization of the effector protein, guide nucleic acid, or the reporter into the polymer matrix may result in less undesired release of the reporter (e.g., during an assay, a measurement, or on the shelf), and thus may cause less background signal, than other immobilization strategies (e.g., conjugation to a pre-formed hydrogel, bead, etc.). In at least some instances this may be due to better incorporation of reporters into the polymer matrix as a copolymer and fewer “free” reporter molecules retained on the hydrogel via non-covalent interactions or non-specific binding interactions.

In some embodiments, a plurality of oligomers and a plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for free-floating programmable nucleases to diffuse into the hydrogel and access immobilized internal reporter molecules. The relative percentages and/or molecular weights of the oligomers may be varied to vary the pore size of the hydrogel. For example, pore size may be tailored to increase the diffusion coefficient of the effector proteins.

In some embodiments, the functional groups attached to the reporters and/or guide nucleic acids may be selected to preferentially incorporate the reporters and/or guide nucleic acids into the polymer matrix via covalent binding at the functional group versus other locations along the nucleic acid backbone of the reporter and/or guide nucleic acid. In some embodiments, the functional groups attached to the reporters and/or guide nucleic acids may be selected to favorably transfer free radicals from the functionalized ends of polymerizable oligomers to the functional group on the end of the reporter and/or guide nucleic acid (e.g., 5′ end), thereby forming a covalent bond and immobilizing the reporter and/or guide nucleic acid rather than destroying other parts of the reporter and/or guide nucleic acid molecules, respectively. In some embodiments, the functional group may comprise a single stranded nucleic acid, a double stranded nucleic acid, an acrydite group, a 5′ thiol modifier, a 3′ thiol modifier, an amine group, a I-LinkerTM group, methacryl group, or any combination thereof. One of ordinary skill in the art will recognize that a variety of functional groups may be used depending on the desired properties of the immobilized components.

VIII. Methods of Making Polymer Matrices with Immobilized Reporters

In some embodiments, a polymer immobilization matrix used in the compositions and methods disclosed herein may comprise a plurality of immobilized programmable nuclease-based detection (e.g., DETECTR) reaction components. The detection reaction components may comprise one or more reporters, one or more programmable nucleases, and/or one or more guide nucleic acids. In some embodiments, the polymer matrix may comprise a hydrogel. In an exemplary embodiment, a plurality of reporters may be immobilized within a hydrogel matrix. In some embodiments, methods of immobilizing a reporter and/or other nucleic acid detection reaction component may comprise (a) providing a polymerizable composition comprising: (i) a plurality of oligomers, (ii) a plurality of polymerizable (e.g., functionalized) oligomers, (iii) a set of polymerizable (e.g., functionalized) reporters (and/or other detection reaction components), and (iv) a set of polymerization initiators; and (b) initiating the polymerization reaction by providing an initiation stimulus. Such components can be utilized in a detection method described herein. For example, the components can be utilized in a single one-pot detection reactions as described herein. In some cases, a buffer used in a single one-pot detection reaction disclosed herein does not comprise TIPP.

Co-polymerization of the reporter into the hydrogel may result in a higher density of reporter/unit volume or reporter/unit area than other immobilization methods utilizing surface immobilization (e.g., onto beads). Co-polymerization of the reporter into the hydrogel may result in less undesired release of the reporter (e.g., during an assay, a measurement, or on the shelf), and thus may cause less background signal, than other immobilization strategies (e.g., conjugation to a pre-formed hydrogel, bead, etc.). In at least some instances this may be due to better incorporation of reporters into the hydrogel as a copolymer and fewer “free” reporter molecules retained on the hydrogel via non-covalent interactions or non-specific binding interactions.

In some embodiments, the plurality of oligomers and the plurality of polymerizable oligomers may comprise an irregular or non-uniform mixture. The irregularity of the mixture of polymerizable oligomers and unfunctionalized oligomers may allow pores to form within the hydrogel (i.e., the unfunctionalized oligomers may act as a porogen). For example, the irregular mixture of oligomers may result in phase separation during polymerization that allows for the generation of pores of sufficient size for programmable nucleases to diffuse into the hydrogel and access internal reporter molecules. The relative percentages and/or molecular weights of the oligomers may be varied to vary the pore size of the hydrogel. For example, pore size may be tailored to increase the diffusion coefficient of the programmable nucleases.

In some embodiments, the functional groups attached to the reporters may be selected to preferentially incorporate the reporters into the hydrogel matrix via covalent binding at the functional group versus other locations along the nucleic acid of the reporter. In some embodiments, the functional groups attached to the reporters may be selected to favorably transfer free radicals from the functionalized ends of polymerizable oligomers to the functional group on the end of the reporter (e.g., 5′ end), thereby forming a covalent bond and immobilizing the reporter rather than destroying other parts of the reporter molecules.

In some embodiments, the polymerizable composition may further comprise one or more polymerizable nucleic acids. In some embodiments, the polymerizable nucleic acids may comprise guide nucleic acids. In some embodiments, the polymerizable nucleic acids may comprise linker or tether nucleic acids. In some embodiments, the polymerizable nucleic acids may be configured to bind to a programmable nuclease. In some embodiments, the programmable nuclease may be immobilized in the polymer matrix.

In some embodiments, the oligomers may form a polymer matrix comprising a hydrogel. In some embodiments, the oligomers may comprise poly(ethylene glycol) (PEG), poly(siloxane), poly(hydroxyethyl acrylate, poly(acrylic acid), poly(vinyl alcohol), poly(butyl acrylate), poly(2-ethylhexyl acrylate), poly(methyl acrylate), poly(ethyl acrylate), poly(acrylonitrile), poly(methyl methacrylate), poly(acrylamide), poly(TMPTA methacrylate), chitosan, alginate, or the like, or any combination thereof. One of ordinary skill in the art will recognize that the oligomers may comprise any oligomer or mix of oligomers capable of forming a hydrogel.

In some embodiments, the oligomers may comprise polar monomers, nonpolar monomers, protic monomers, aprotic monomers, solvophobic monomers, or solvophillic monomers, or any combination thereof.

In some embodiments, the oligomers may comprise a linear topology, branched topology, star topology, dendritic topology, hyperbranched topology, bottlebrush topology, ring topology, catenated topology, or any combination thereof. In some embodiments, the oligomers may comprise 3-armed topology, 4-armed topology, 5-armed topology, 6-armed topology, 7-armed topology, 8-armed topology, 9-armed topology, or 10-armed topology.

In some embodiments, the oligomers may comprise at least about 2 monomers, at least about 3 monomers, at least about 4 monomers, at least about 5 monomers, at least about 6 monomers, at least about 7 monomers, at least about 8 monomers, at least about 9 monomers, at least about 10 monomers, at least about 20 monomers, at least about 30 monomers, at least about 40 monomers, at least about 50 monomers, at least about 60 monomers, at least about 70 monomers, at least about 80 monomers, at least about 90 monomers, at least about 100 monomers, at least about 200 monomers, at least about 300 monomers, at least about 400 monomers, at least about 500 monomers, at least about 600 monomers, at least about 700 monomers, at least about 800 monomers, at least about 900 monomers, at least about 1000 monomers, at least about 2000 monomers, at least about 3000 monomers, at least about 4000 monomers, at least about 5000 monomers, at least about 6000 monomers, at least about 7000 monomers, at least about 8000 monomers, at least about 9000 monomers, at least about 10000 monomers, at least about 20000 monomers, at least about 30000 monomers, at least about 40000 monomers, at least about 50000 monomers, at least about 60000 monomers, at least about 70000 monomers, at least about 80000 monomers, at least about 90000 monomers, or at least about 100000 monomers.

In some embodiments, the oligomers may comprise a homopolymer, a copolymer, a random copolymer, a block copolymer, an alternative copolymer, a copolymer with regular repeating units, or any combination thereof.

In some embodiments, the oligomers may comprise 1 type of monomer, 2 types of monomers, 3 types of monomers, 4 types of monomers, 5 types of monomers, 6 types of monomers, 7 types of monomers, 8 types of monomers, 9 types of monomers, or 10 types of monomers.

The polymerizable oligomers may comprise any of the oligomers described herein. In some embodiments, the polymerizable oligomers may comprise one or more functional groups. In some embodiments, the functional group may comprise an acrylate group, N-hydroxysuccinimide ester group, thiol group, carboxyl group, azide group, alkyne group, an alkene group, or any combination thereof. One of ordinary skill in the art will recognize that a variety of functional groups may be used to functionalize oligomers into polymerizable oligomers depending on the desired properties of the polymerizable oligomers.

In some embodiments, the polymerizable oligomers may form a polymer matrix comprising a hydrogel. In some embodiments, the polymerizable oligomers may comprise PEG, poly(siloxane), poly(hydroxyethyl acrylate, poly(acrylic acid), poly(vinyl alcohol), or any combination thereof. One of ordinary skill in the art will recognize that the set of polymerizable oligomers may comprise any polymer capable of forming a hydrogel.

In some embodiments, the set of polymerizable oligomers comprises polar monomers, nonpolar monomers, protic monomers, aprotic monomers, solvophobic monomers, or solvophillic monomers.

In some embodiments, the set of polymerizable oligomers comprises a linear topology, branched topology, star topology, dendritic topology, hyperbranched topology, bottlebrush topology, ring topology, catenated topology, or any combination thereof. In some embodiments, the set of polymerizable oligomers comprises 3-armed topology, 4-armed topology, 5-armed topology, 6-armed topology, 7-armed topology, 8-armed topology, 9-armed topology, or 10-armed topology.

In some embodiments, the set of polymerizable oligomers comprises at least about 2 monomers, at least about 3 monomers, at least about 4 monomers, at least about 5 monomers, at least about 6 monomers, at least about 7 monomers, at least about 8 monomers, at least about 9 monomers, at least about 10 monomers, at least about 20 monomers, at least about 30 monomers, at least about 40 monomers, at least about 50 monomers, at least about 60 monomers, at least about 70 monomers, at least about 80 monomers, at least about 90 monomers, at least about 100 monomers, at least about 200 monomers, at least about 300 monomers, at least about 400 monomers, at least about 500 monomers, at least about 600 monomers, at least about 700 monomers, at least about 800 monomers, at least about 900 monomers, at least about 1000 monomers, at least about 2000 monomers, at least about 3000 monomers, at least about 4000 monomers, at least about 5000 monomers, at least about 6000 monomers, at least about 7000 monomers, at least about 8000 monomers, at least about 9000 monomers, at least about 10000 monomers, at least about 20000 monomers, at least about 30000 monomers, at least about 40000 monomers, at least about 50000 monomers, at least about 60000 monomers, at least about 70000 monomers, at least about 80000 monomers, at least about 90000 monomers, or at least about 100000 monomers. As used herein, “about” may mean plus or minus 1 monomer, plus or minus 10 monomers, plus or minus 100 monomers, plus or minus 1000 monomers, plus or minus 10000 monomers, or plus or minus 100000 monomers.

In some embodiments, the set of polymerizable oligomers comprises a homopolymer, a copolymer, a random copolymer, a block copolymer, an alternative copolymer, a copolymer with regular repeating units, or any combination thereof.

In some embodiments, the set of polymerizable oligomers comprises 1 type of monomer, 2 types of monomers, 3 types of monomers, 4 types of monomers, 5 types of monomers, 6 types of monomers, 7 types of monomers, 8 types of monomers, 9 types of monomers, or 10 types of monomers.

In some embodiments, the polymerizable composition may comprise a mix of unfunctionalized or unmodified oligomers and polymerizable oligomers as described herein. In some embodiments, the unfunctionalized or unmodified oligomers may act as porogens to generate pores within the polymer matrix.

The polymerizable reporters may comprise any of the reporters described herein. In some embodiments, the set of polymerizable reporters may comprise one or more functional groups. In some embodiments, the functional group may comprise a single stranded nucleic acid, a double stranded nucleic acid, an acrydite group, a 5′ thiol modifier, a 3′ thiol modifier, an amine group, a I-LinkerTM group, methacryl group, or any combination thereof. One of ordinary skill in the art will recognize that a variety of functional groups may be used with the set of polymerizable reporters depending on the desired properties of the polymerizable reporters.

In some embodiments, the set of initiators may comprise one or more photoinitiators or thermal initiators. In some embodiments, the set of initiators may comprise cationic initiators, anionic initiators, or radical initiators. In some embodiments, the set of initiators may comprise AIBN, AMBN, ADVN, ACVA, dimethyl 2,2′-azo-bis(2methylpropionate), AAPH, 2,2′-azobis[2-(2-imidazolin-2-yl)-propane]dihydrochloride, TBHP, cumene hydroperoxide, di-tert-butyl peroxide, dicumyl peroxide, BPO, dicyandamide, cyclohexyl tosylate, diphenyl(methyl)sulfonium tetrafluoroborate, benzyl(4-hydroxyphenyl)-methylsulfonium hexafluoroantimonate, (4-hydroxyphenyl)methyl-(2-methylbenzyl)sulfonium hexafluoroantimonate, camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis(diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone, 1-benzoylchclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylantraquinone, sodium anthraquinone-2-sulfonate monohydrate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′bis(2-chlorophenyl)-4,4′,5,5′-tetraphenyl-1,2′-biimidazole, diphenyl(2,4,6-trimethyl-benzoyl)phosphine oxide, phenylbis(2,4,6-trimethyl-benzoyl)phosphine oxide, lithium phenyl(2,4,6-trimethylbenzoyl)phosphinate, diphenyliodonium trifluoromethanesulfonate, diphenyliodonium hexafluorophosphate, diphenyliodonium hexafluoroarsenate, bis(4-tert-butylphenyl)-iodonium triflate, bis(4-tert-butylphenyl)iodonium hexafluorophosphate, 4-isopropyl-4′-methyl-diphenyliodonium tetrakis(pentafluorophenyl)borate, [4-[(2-hydroxytetradecyl)-oxy]phenyl]phenyliodonium hexafluoroantimonate, bis[4-(tert-butyl)phenyl]-iodonium tetra(nonafluoro-tert-butoxy)aluminate, cyclopropyldiphenylsulfonium tetrafluoroborate, triphenylsulfonium bromide, triphenylsulfonium tetrafluoroborate, tri-p-tolylsulfonium triflate, tri-p-tolylsulfonium hexafluorophosphate, 4-nitrobenzenediazonium tetrafluoroborate, 2-(4-methoxyphenyl)-4,6-bis(trichloromethyl)-1,3,5-triazine, 2-(1,3-benzodioxol-5-yl)-4,6-bis(trichloromethyl)-1,3,5-triazine, 2-(4-methoxystyryl)-4,6-bis(trichloromethyl)-1,3,5-triazine, 2-(3,4-dimethoxystyryl)-4,6-bis(trichloromethyl)-1,3,5-triazine, 2-[2-(Furan-2-yl)vinyl]-4,6-bis(trichloromethyl)-1,3,5-triazine, 2-[2-(5-methylfuran-2-yl)vinyl]-4,6-bis(trichloromethyl)-1,3,5-triazine, 2-(9-oxoxanthen-2-yl)proprionic acid 1,5,7-triazabicyclo[4.4.0]dec-5-ene salt, 2-(9-oxoxanthen-2-yl)proprionic acid 1,5-diazabicyclo[4.3.0]non-5-ene salt, 2-(9-oxoxanthen-2-yl)proprionic acid 1,8-diazabicyclo[5.4.0]-undec-7-ene salt, acetophenone O-benzoyloxime, 2-nitrobenzyl cyclohexylcarbamate, 1,2-bis(4-methoxyphenyl)-2-oxoethyl cyclohexylcarbamate, tert-amyl peroxybenzoate, 4,4-azobis(4-cyanovaleric acid), 1,1′-azobis(cyclohexanecarbonitrile), 2,2′-azobisisobutyronitrile, benzoyl peroxide, 2,2-bi(tert-butylperoxy)butane, 1,1-bis(tert-butylperoxy)cyclohexane, 2,5-bis(tert-butylperoxy)-2,5-dimethylhexane, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, 2-Hydroxy-2-methylpropiophenone, or any combination thereof. One of ordinary skill in the art will recognize that a variety of initiators may be used depending on the desired reaction conditions and chemistries.

In some embodiments, the initiation stimulus is UV light. In some embodiments, the initiation stimulus is UV light through a photomask. In some embodiments, the initiation stimulus is heat.

In some embodiments, the hydrogel may comprise a circular cross-sectional shape, a rectangular cross-sectional shape, a star cross-sectional shape, a dollop shape, an amorphous shape, or any shape of interest, or any combination thereof.

In some embodiments, a mask may be used to shape the initiation stimulus deposition on the polymerizable components (e.g., oligomers, etc.) and thereby shape the resulting polymer matrix. In some embodiments, the mask may comprise a circular shape, a rectangular shape, a star shape, a dollop shape, an amorphous shape, or any shape of interest, or any combination thereof.

IX. Hydrogel Compositions with Immobilized Reporters

Provided herein are compositions comprising hydrogels comprising immobilized reporters. In some aspects, provided herein are compositions comprising a hydrogel comprising (a) a network of covalently bound oligomers and (b) immobilized reporters covalently bound to said network.

In some cases, an exemplary hydrogel comprises a plurality of reporters co-polymerized with a plurality of oligomers (modified and unmodified) to form a network or matrix. Exemplary multiplexing schemes utilizing hydrogel-immobilized reporters may be implemented in any of the devices or methods described herein. Multiplexing could be distinguished through spatial multiplexing by knowing the location of hydrogels functionalized with each guide nucleic acid and/or through shape, by using different shapes of hydrogel for each guide nucleic acid.

In some embodiments, the composition may comprise a hydrogel comprising (a) a polymer network comprising covalently bound oligomers co-polymerized with reporters to covalently bind and immobilize the reporters to said network, and (b) immobilized programmable nuclease complexes covalently bound to said network (e.g., via co-polymerization or after reporter-immobilized polymer formation), wherein said programmable nuclease complexes may comprise a programmable nuclease and a guide nucleic acid. In some embodiments, the guide nucleic acid and/or the programmable nuclease may be immobilized to or in the hydrogel as described herein (e.g., during or after formation of the hydrogel).

In some embodiments, the network of covalently bound oligomers may comprise a network formed by polymerizing one or more PEG species. In some embodiments, the network of covalently bound oligomers may comprise a network formed by polymerizing PEG comprising acrylate functional groups. In some embodiments, the acrylate functional groups may be PEG end groups. In some embodiments, the network may be formed by polymerizing PEG comprising acrylate functional groups with unmodified PEG. The molecular weight of the acrylate-modified PEG (e.g., PEG-diacrylate) and the unmodified PEG may be the same or different.

In some embodiments, the network of covalently bound oligomers may comprise a network formed from polymerizing one or more PEG species, wherein each PEG species may comprise a linear topology, branched topology, star topology, dendritic topology, hyperbranched topology, bottlebrush topology, ring topology, catenated topology, or any combination thereof. In some embodiments, the network of covalently bound oligomers may comprise a network formed from polymerizing one or more PEG species comprising a 3-armed topology, a 4-armed topology, a 5-armed topology, a 6-armed topology, a 7-armed topology, a 8-armed topology, a 9-armed topology, or a 10-armed topology.

In some embodiments, the immobilized reporter may comprise a reporter molecule covalently bound to a linker molecule, wherein the linker molecule is covalently bound to the hydrogel (e.g., via co-polymerization with the oligomers as described herein). In some embodiments, the linker molecule may comprise a single stranded nucleic acid, a double stranded nucleic acid, an acrydite group, a 5′ thiol modifier, a 3′ thiol modifier, an amine group, a I-LinkerTM group, or any combination thereof. One of ordinary skill in the art will recognize that a variety of linker molecules may be used.

In some cases, the immobilized guide nucleic acid may comprise a guide nucleic acid covalently bound to a linker molecule, wherein the linker molecule is covalently bound to the hydrogel. In some embodiments, the linker molecule may comprise a single stranded nucleic acid, a double stranded nucleic acid, an acrydite group, a 5′ thiol modifier, a 3′ thiol modifier, an amine group, a I-LinkerTM group, or any combination thereof. One of ordinary skill in the art will recognize that a variety of linker molecules may be used.

In some cases, the immobilized programmable nuclease may comprise a programmable nuclease covalently bound to a linker molecule, wherein the linker molecule is covalently bound to the hydrogel. In some embodiments, the linker molecule may comprise a single stranded nucleic acid, a double stranded nucleic acid, an acrydite group, a 5′ thiol modifier, a 3′ thiol modifier, an amine group, a I-LinkerTM group, or any combination thereof. One of ordinary skill in the art will recognize that a variety of linker molecules may be used.

X. Methods of Using Hydrogels with Immobilized Reporters

Any of the methods described herein may utilize hydrogels with immobilized reporters for target detection assays. For example, hydrogels with immobilized reporters can be utilized in a one-pot detection (e.g., DETECTR) reaction of the present disclosure. In some embodiments, the hydrogel comprises (a) a network of covalently bound oligomers and (b) immobilized reporters covalently bound to said network. A solution comprising target nucleic acid molecules and programmable nuclease complexes may be applied to the hydrogel (e.g., by pipetting or flowing over the hydrogel). The immobilized reporters may comprise a nucleic acid with a sequence cleavable by the programmable complex when the programmable nuclease complex is activated by binding of its associated guide nucleic acid to a target nucleic acid molecule as described herein. When activated, the programmable nuclease complex may trans-cleave the cleavable nucleic acid of the reporter molecule and generates a detectable signal as described herein. For example, the reporter may comprise a detection moiety which may be release upon cleavage of the reporter as described herein. The detection moiety may comprise FAM-biotin which may be captured by one or more capture molecules coupled to a surface of a support (e.g., a lateral flow assay strip) at a detection location as described herein. Detection of the detectable signal generated at the detection location by the detection moiety may indicate the presence of the target nucleic acid in the sample as described herein. In some cases, a buffer used in a single one-pot detection reaction disclosed herein does not comprise TIPP.

Any of the multiplexing methods described herein may utilize hydrogels with immobilized reporters for multiplexed target detection assays. In some embodiments, each hydrogel may comprise (a) a polymer network of covalently bound oligomers co-polymerized with reporters to covalently bind and immobilize the reporters to said network, and (b) one or more immobilized programmable nuclease complexes covalently bound to said network. Each of the programmable nuclease complexes may comprise a programmable nuclease and a guide nucleic acid. In some embodiments, the guide nucleic acid and/or the programmable nuclease may be immobilized to or in the hydrogel as described herein (e.g., during or after formation of the hydrogel). In some embodiments, multiplexing for a plurality of different targets may be facilitated by providing a plurality different and/or spatially separated hydrogels comprising a plurality of different programmable nuclease-based detection reaction components. In some embodiments, each hydrogel may comprise a different programmable nuclease as described herein. Alternatively, or in combination, each hydrogel may comprise a different guide nucleic acid configured to bind to a different target nucleic acid sequence as described herein. Alternatively, or in combination, each hydrogel may comprise a different reporter as described herein. Alternatively, or in combination, each hydrogel may comprise a different shape and be deposited on a surface of a support at different detection locations. For example, a first hydrogel may comprise a first programmable nuclease, a first guide nucleic acid configured to bind a first target nucleic acid, and a first reporter. A second hydrogel may comprise a second programmable nuclease, a second guide nucleic acid configured to bind a second target nucleic acid, and a second reporter. A third hydrogel may comprise a third programmable nuclease, a third guide nucleic acid configured to bind a third target nucleic acid, and a third reporter. The programmable nucleases may be the same programmable nuclease or different programmable nuclease. The guide nucleic acids may be different guide nucleic acids configured to recognize different target nucleic acids. The reporters may be the same reporter or different reporters. A solution comprising one or more target nucleic acid molecules may be applied to the hydrogels, e.g., by pipetting or flowing over the hydrogels. The immobilized reporters may comprise a nucleic acid with a sequence cleavable by the programmable nuclease complexes when the programmable nuclease complexes are activated by binding of their respective guide nucleic acids to their respective target nucleic acid molecules as described herein. When activated, the programmable nuclease complexes may trans-cleave the cleavable nucleic acid of the reporter molecule and generates a detectable signal at the detection location as described herein. For example, the reporter may comprise a detection moiety which may be release upon cleavage of the reporter as described herein. The detection moiety may comprise FAM-biotin, which may be captured by one or more capture molecules coupled to a surface of a support (e.g., a lateral flow assay strip) at a detection location as described herein. Alternatively, the detection moiety may comprise a quencher moiety which may be released from the hydrogel upon cleavage of the reporter, thereby allowing a fluorescent moiety on the other end of the reporter to fluoresce at the detection location comprising the hydrogel. Detection of the detectable signal generated at the detection locations by the detection moiety may indicate the presence of the target nucleic acid in the sample as described herein. Each hydrogel may have a different shape and detection of a target nucleic acid may comprise detecting a particular fluorescent shape corresponding to the hydrogel shape at the detection location.

XI. Devices Comprising Hydrogels with Immobilized Reporters

Any of the systems or devices described herein may comprise one or more hydrogels with immobilized reporters.

In some embodiments, the systems and devices described herein may comprise a plurality of hydrogels each comprising reporter molecules (e.g., in order to facilitate multiplexing and/or improve signal). In some embodiments, a first hydrogel may comprise a shape different from a shape of a second hydrogel. In some embodiments, the first hydrogel may comprise a plurality of first reporter molecules different from a plurality of second reporter molecules of the second hydrogel. In some embodiments, the reporters are the same in the first and second hydrogels. In some embodiments, the first hydrogel may comprise a circular shape, a square shape, a star shape, or any other shape distinguishable from a shape of the second hydrogel. In some embodiments, the plurality of first reporter molecules may each comprise a sequence cleavable by a programmable nuclease complex comprising a first programmable nuclease and a first guide nucleic acid. In some embodiments, the plurality of second reporter molecules may each comprise a sequence not cleavable by the first programmable nuclease complex.

Any of the systems or devices described herein may comprise a plurality of hydrogels each comprising reporter molecules. For example, a first hydrogel may comprise a plurality of first reporter molecules different from a plurality of second reporter molecules of a second hydrogel. In some embodiments, the plurality of first reporter molecules may each comprise a first fluorescent moiety, wherein the first fluorescent moiety is different than second fluorescent moieties of in each of the plurality of second reporter molecules. In some embodiments, the plurality of first reporter molecules may each comprise a sequence cleavable by a first programmable nuclease complex comprising a first programmable nuclease and a first guide nucleic acid. In some embodiments, the plurality of second reporter molecules may each comprise a sequence cleavable by a second programmable nuclease complex comprising a second programmable nuclease and a second guide nucleic acid.

Any of the systems or devices described herein may comprise at least about 2 hydrogels, at least about 3 hydrogels, at least about 4 hydrogels, at least about 5 hydrogels, at least about 6 hydrogels, at least about 7 hydrogels, at least about 8 hydrogels, at least about 9 hydrogels, at least about 10 hydrogels, at least about 20 hydrogels, at least about 30 hydrogels, at least about 40 hydrogels, at least about 50 hydrogels, at least about 60 hydrogels, at least about 70 hydrogels, at least about 80 hydrogels, at least about 90 hydrogels, at least about 100 hydrogels, at least about 200 hydrogels, at least about 300 hydrogels, at least about 400 hydrogels, at least about 500 hydrogels, at least about 600 hydrogels, at least about 700 hydrogels, at least about 800 hydrogels, at least about 900 hydrogels, at least about 1000 hydrogels.

Any of the systems or devices described herein may comprise one or more compartments, chambers, channels, or locations comprising the one or more hydrogels. In some embodiments, two or more of the compartments may be in fluid communication, optical communication, thermal communication, or any combination thereof with one another. In some embodiments, two or more compartments may be arranged in a sequence. In some embodiments, two or more compartments may be arranged in parallel. In some embodiments, two or more compartments may be arranged in sequence, parallel, or both. In some embodiments, one or more compartments may comprise a well. In some embodiments, one or more compartments may comprise a flow strip. In some embodiments, one or more compartments may comprise a heating element.

In some embodiments, the device may be a handheld device. In some embodiments, the device may be point-of-need device. In some embodiments, the device may comprise any one of the device configurations described herein. In some embodiments, the device may comprise one or more parts of any one of the device configurations described herein.

### XII. Methods of Nucleic Acid Editing

Provided herein are methods of editing target nucleic acids. In general, editing refers to modifying the nucleobase sequence of a target nucleic acid. However, compositions and systems disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid. Methods of editing may comprise contacting a target nucleic acid with an effector protein and an engineered guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to any one of SEQ ID NOs: 1-4. Methods of editing may comprise contacting a target nucleic acid with an effector protein and an engineered guide nucleic acid, wherein the target nucleic acid comprises a PAM sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein N is A, G, C, or T, and wherein Y is a C or T. Methods may also comprise contacting a target cell with an effector protein and an engineered guide nucleic acid, wherein the engineered guide nucleic acid comprises a nucleobase sequence that is at least 75%, at least 80%, at least 85%, at least 90%, at least 95%, at least 98%, at least 99% or 100% identical to any one of SEQ ID NOs: 19-21, as provided in TABLE 2.

Editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may modify the target nucleic acid. Modifying the target nucleic acid comprises cleaving the target nucleic acid, deleting a nucleotide of the target nucleic acid, inserting a nucleotide into the target nucleic acid, substituting a nucleotide of the target nucleic acid with a donor nucleotide or an additional nucleotide, or any combination thereof. In some cases, modifying the target nucleic acid may comprise cleaving the target nucleic acid. In some cases, modifying the target nucleic acid may comprise deleting a nucleotide of the target nucleic acid. In some cases, modifying the target nucleic acid may comprise inserting a nucleotide into the target nucleic acid. In some cases, modifying the target nucleic acid may comprise substituting a nucleotide of the target nucleic acid with a donor nucleotide or an additional nucleotide.

Editing may comprise single stranded cleavage, double stranded cleavage, donor nucleic acid insertion, epigenetic modification (e.g., methylation, demethylation, acetylation, or deacetylation), or a combination thereof. In some instances, cleavage (single-stranded or double-stranded) is site-specific, meaning cleavage occurs at a specific site in the target nucleic acid, often within the region of the target nucleic acid that hybridizes with the engineered guide nucleic acid spacer region. In some cases, effector proteins introduce a single-stranded break in a target nucleic acid to produce a cleaved nucleic acid. In some cases, the effector protein is capable of introducing a break in a single stranded RNA (ssRNA). The effector protein may be coupled to an engineered guide nucleic acid that targets a particular region of interest in the ssRNA. In some instances, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor template, such that the repair results in an insertion-deletion (indel) in the target nucleic acid at or near the site of the double-stranded break.

In some instances, the effector protein is fused to a chromatin-modifying enzyme. In some cases, the fusion protein chemically modifies the target nucleic acid, for example by methylating, demethylating, or acetylating the target nucleic acid in a sequence specific or non-specific manner.

Methods may comprise use of two or more effector proteins. An illustrative method for introducing a break in a target nucleic acid comprises contacting the target nucleic acid with: (a) a first engineered guide nucleic acid comprising a region that binds to a first effector protein comprising at least 75% sequence identity to a sequence selected from the group consisting of SEQ ID NOs: 1-4; and (b) a second engineered guide nucleic acid comprising a region that binds to a second effector protein comprising at least 75% sequence identity to a sequence selected from the group consisting of SEQ ID NOs: 1-4 wherein the first engineered guide nucleic acid comprises an additional region that binds to the target nucleic acid and wherein the second engineered guide nucleic acid comprises an additional region that binds to the target nucleic acid.

In some embodiments, editing a target nucleic acid comprises genome editing. Genome editing may comprise modifying a genome, chromosome, plasmid, or other genetic material of a cell or organism. In some embodiments the genome, chromosome, plasmid, or other genetic material of the cell or organism is modified in vivo. In some embodiments the genome, chromosome, plasmid, or other genetic material of the cell or organism is modified in a cell. In some embodiments the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may be inserted into the target nucleic acid.

In some instances, methods comprise inserting a donor nucleic acid into a cleaved target nucleic acid. The donor nucleic acid may be inserted at a specified (e.g., effector-targeted) point within the target nucleic acid. In some instances, methods comprise contacting a target nucleic acid with an effector protein comprising an amino acid sequence that is at least 70%, at least 75%, at least 80%, at least 85%, at least 90%, at least 95%, at least 97%, at least 98%, at least 99%, or 100% identical to any one of SEQ ID NOs: 1-4, thereby introducing a single-stranded break in the target nucleic acid; contacting the target nucleic acid with a second effector, optionally comprising an amino acid sequence that is at least 70%, at least 75%, at least 80%, at least 85%, at least 90%, at least 95%, at least 97%, at least 98%, or at least 99% identical to any one of SEQ ID NOs: 1-4, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally via HDR or NHEJ, thereby introducing a new sequence into the target nucleic acid (e.g., at a cleavage site or in between two cleavage sites).

In some cases, methods comprise editing a target nucleic acid with two or more programmable nickases. Editing a target nucleic acid may comprise introducing a two or more single-stranded breaks in a target nucleic acid. In some embodiments, a break may be introduced by contacting a target nucleic acid with a programmable nickase and an engineered guide nucleic acid. The engineered guide nucleic acid may bind to the programmable nickase and hybridize to a region of the target nucleic acid, thereby recruiting the programmable nickase to the region of the target nucleic acid. Binding of the programmable nickase to the engineered guide nucleic acid and the region of the target nucleic acid may activate the programmable nickase, and the programmable nickase may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first programmable nickase and hybridizes to a first region of the target nucleic acid and a second guide nucleic acid that binds to a second programmable nickase and hybridizes to a second region of the target nucleic acid. The first programmable nickase may introduce a first break in a first strand at the first region of the target nucleic acid, and the second programmable nickase may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

In some cases, editing is achieved by fusing an effector protein such as an effector protein to a heterologous sequence. The heterologous sequence may be a suitable fusion partner, e.g., a protein that provides recombinase activity by acting on the target nucleic acid sequence. In some embodiments, the fusion protein comprises an effector protein such as an effector protein fused to a heterologous sequence by a linker. The heterologous sequence or fusion partner may be a base editing domain. The base editing domain may be an ADAR1/2 or any functional variant thereof. The heterologous sequence or fusion partner may be fused to the C-terminus, N-terminus, or an internal portion (e.g., a portion other than the N- or C-terminus) of the effector protein. The heterologous sequence or fusion partner may be fused to the effector protein by a linker. A linker may be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

### A. Donor Nucleic Acids

In some cases, modifying the target nucleic acid may comprise contacting the target nucleic acid with the donor nucleic acid. Donor nucleic acids of any suitable size may be integrated into a target nucleic acid or genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some instances, donor nucleic acids are more than 500 kilobases (kb) in length.

The donor nucleic acid may comprise a sequence that is derived from a plant, bacteria, virus or an animal. The animal may be human. The animal may be a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). The non-human animal may be a domesticated mammal or an agricultural mammal.

In some cases, a composition disclosed herein further comprises a donor nucleic acid. In some cases, a vector disclosed herein further comprises a donor nucleic acid or comprises a nucleic acid encoding a donor nucleic acid.

In some cases, a vector disclosed herein encoding an effector protein and an engineered guide nucleic acid further comprises or encodes a donor nucleic acid. In some cases, a vector system encoding a first vector encoding an effector protein and a second vector encoding an engineered guide nucleic acid further includes a third vector comprising or encoding a donor nucleic acid.

In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome. As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid resulting in a nick or double strand break—nuclease activity). As yet another example, when used in reference to homologous recombination, the term donor nucleic acid refers to a sequence of DNA that serves as a template in the process of homologous recombination, which may carry the modification that is to be or has been introduced into the target nucleic acid. By using this donor nucleic acid as a template, the genetic information, including the modification, is copied into the target nucleic acid by way of homologous recombination.

### B. Genetically Modified Cells and Organisms

Methods of editing described herein may be employed to generate a genetically modified cell. The genetically modified cell may be a recombinant cell. The edited cells/genetically modified cells/recombinant cells may form a population. The cell may be a eukaryotic cell (e.g., a mammalian cell) or a prokaryotic cell (e.g., an archaeal cell). The cell may be derived from a multicellular organism and cultured as a unicellular entity. The cell may comprise a heritable genetic modification, such that progeny cells derived therefrom comprise the heritable genetic mutation. The cell may be progeny of a genetically modified cell comprising a genetic modification of the genetically modified parent cell. A genetically modified cell may comprise a deletion, insertion, mutation, or non-native sequence relative to a wild-type version of the cell or the organism from which the cell was derived.

Methods of generating a recombinant cell may comprise contacting a target cell with an effector protein and an engineered guide nucleic acid, wherein the effector protein comprises an amino acid sequence that is at least 70%, at least 75%, at least 80%, at least 85%, at least 90%, at least 95%, at least 97%, at least 98%, or at least 99% identical to any one of SEQ ID NOs: 1-4. Methods may comprise contacting a target cell with an effector protein and an engineered guide nucleic acid, wherein the amino acid sequence of the effector protein is at least 70%, at least 75%, at least 80%, at least 85%, at least 90%, at least 95%, at least 97%, at least 98%, or at least 99% identical to any one of SEQ ID NOs: 1-4. Methods may comprise contacting a target cell with an effector protein and an engineered guide nucleic acid, wherein the target nucleic acid comprises a PAM sequence directly adjacent to a target sequence of a target nucleic acid, the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein N is adenine A, G, C, or T, and wherein Y is a C or T. Methods may also comprise contacting a target cell with an effector protein and an engineered guide nucleic acid, wherein the engineered guide nucleic acid comprises a nucleobase sequence that is at least 75%, at least 80%, at least 85%, at least 90%, at least 95%, at least 98%, at least 99% or 100% identical to any one of SEQ ID NOs: 19-21, as provided in TABLE 2.

Methods may comprise contacting a target cell with a nucleic acid (e.g., a plasmid or mRNA) comprising a nucleobase sequence encoding an effector protein, wherein the effector protein comprise comprises an amino acid sequence that is at least 70%, at least 75%, at least 80%, at least 85%, at least 90%, at least 95%, at least 97%, at least 98%, or at least 99% identical to any one of SEQ ID NOs: 1-4. In some cases, the nucleic acid may also encode the engineered guide nucleic acid. Methods may comprise contacting target cells with a nucleic acid (e.g., a plasmid or mRNA) comprising a nucleobase sequence encoding an engineered guide nucleic acid, a tracrRNA, a crRNA, or any combination thereof.

Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. In some cases, the nanoparticle delivery comprises lipid nanoparticle delivery or gold nanoparticle delivery. In some cases, the nanoparticle delivery comprises lipid nanoparticle delivery. In some cases, the nanoparticle delivery comprises gold nanoparticle delivery.

Providing the effector protein (or the nucleic acid encoding the effector protein) and the engineered guide nucleic acid to the target cell may generate a double-stranded break in the genome of the target cell. In some cases, the method of generating a recombinant cell comprises detecting the double-stranded break. The double-stranded break may be repaired by methods described thereof. In some cases, the repairing results in an indel in the genome of the target cell. In some cases, the method comprises delivering a donor nucleic acid to a target cell. The donor nucleic acid may be incorporated into the genome of the target cell. The method may also comprise detecting the incorporation of the donor nucleic acid in the genome of the target cell.

Methods may comprise cell line engineering (e.g., engineering a cell from a cell line for bioproduction). Cell lines may be used to produce a desired protein. In some embodiments, target nucleic acids comprise a genomic sequence. In some embodiments, the cell line is a Chinese hamster ovary cell line (CHO), human embryonic kidney cell line (HEK), cell lines derived from cancer cells, cell lines derived from lymphocytes, and the like. Non-limiting examples of cell lines includes: C8161, CCRF-CEM, MOLT, mIMCD-3, NHDF, HeLa—S3, Huhl, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-1OT1/2, C6/36, Cal-27, CHO, CHO-7, CHO-IR, CHO—K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR.

Non-limiting examples of cells that may be engineered or modified with compositions and methods described herein include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that may be engineered or modified with compositions and methods described herein include plant cells, such as parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that may be engineered or modified with compositions and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Methods of the disclosure may be performed in a subject. Compositions of the disclosure may be administered to a subject. A subject may be a human. A subject may be a mammal (e.g., rat, mouse, cow, dog, pig, sheep, horse). A subject may be a vertebrate or an invertebrate. A subject may be a laboratory animal. A subject may be suffering from a disease. A subject may display symptoms of a disease. A subject may not display symptoms of a disease, but still have a disease. A subject may be under medical care of a caregiver (e.g., the subject is hospitalized and is treated by a physician). Methods of the disclosure may be performed in a plant, bacteria, or a fungus.

The disclosure also provides effector proteins, compositions and/or complexes disclosed herein for use in therapy.

The disclosure also provides the use of effector proteins, compositions and/or complexes disclosed herein for the manufacture of a medicament.

Methods of the disclosure may be performed in a cell. A cell may be in vitro. A cell may be in vivo. A cell may be ex vivo. A cell may be an isolated cell. A cell may be a cell inside of an organism. A cell may be an organism. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a prokaryotic cell or derived from a prokaryotic cell. A cell may be a bacterial cell or may be derived from a bacterial cell. A cell may be an archaeal cell or derived from an archaeal cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell. A cell may be a microbe cell or derived from a microbe cell. A cell may be a fungi cell or derived from a fungi cell. A cell may be from a specific organ or tissue. In some embodiments, the cell is not a cell prepared from a human embryo.

Methods of the disclosure may be performed in a eukaryotic cell or cell line. In some embodiments, the eukaryotic cell is a Chinese hamster ovary (CHO) cell. In some embodiments, the eukaryotic cell is a Human embryonic kidney 293 cells (also referred to as HEK or HEK 293) cell. Non-limiting examples of cell lines that may be used with compositions, systems and methods of the present disclosure include C8161, CCRF-CEM, MOLT, mIMCD-3, NHDF, HeLa-S3, Huhl, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-1OT1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that may be used with the disclosure include immune cells, such as CART, T-cells, B-cells, NK cells (or natural killer T cells), granulocytes, basophils, eosinophils, neutrophils, mast cells, monocytes, macrophages, dendritic cells, antigen-presenting cells (APC), or adaptive cells. Non-limiting examples of cells that may be used with this disclosure also include plant cells, such as Parenchyma, sclerenchyma, collenchyma, xylem, phloem, germline (e.g., pollen). Cells from lycophytes, ferns, gymnosperms, angiosperms, bryophytes, charophytes, chlorophytes, rhodophytes, or glaucophytes. Non-limiting examples of cells that may be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Further provided are cells comprising an effector protein disclosed herein. Also provided are cells comprising an effector protein disclosed herein and an engineered guide nucleic acid disclosed herein. Also provided are cells comprising complexes disclosed herein.

### C. Agricultural Engineering

Compositions and methods of the disclosure may be used for agricultural engineering. For example, compositions and methods of the disclosure may be used to confer desired traits on a plant. A plant may be engineered for the desired physiological and agronomic characteristic using the present disclosure. In some embodiments, the target nucleic acid sequence comprises a nucleic acid sequence of a plant. In some embodiments, the target nucleic acid sequence comprises a genomic nucleic acid sequence of a plant cell. In some embodiments, the target nucleic acid sequence comprises a nucleic acid sequence of an organelle of a plant cell. In some embodiments, the target nucleic acid sequence comprises a nucleic acid sequence of a chloroplast of a plant cell.

The plant may be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

The plant may be a monocotyledonous plant. Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant may belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant may include algae.

### D. Certain Target Nucleic Acids

Disclosed herein are compositions, systems and methods for detecting and/or modifying a target nucleic acid. In some instances, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be an RNA. The target nucleic acids include but are not limited to mRNA, rRNA, tRNA, non-coding RNA, long non-coding RNA, and microRNA (miRNA). In some instances, the target nucleic acid is complementary DNA (cDNA) synthesized from a single-stranded RNA template in a reaction catalyzed by a reverse transcriptase. In some cases, the target nucleic acid is ss-cDNA. In some cases, the target nucleic acid is ds-cDNA. In some cases, the target nucleic acid is single-stranded RNA (ssRNA) or mRNA.

In some instances, an effector protein recognizes a protospacer adjacent motif (PAM) sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T.

In some cases, an effector protein recognizes a PAM sequence directly adjacent to a target sequence of a target nucleic acid, wherein the PAM sequence is selected from the group consisting of NNNNNYN (SEQ ID NO: 42), NNNNYYN (SEQ ID NO: 43), and NNNNYTN (SEQ ID NO: 44). In some cases, an effector protein recognizes a PAM sequence directly adjacent to a target sequence of a target nucleic acid, wherein the PAM sequence is selected from the group consisting of NNNNNYN (SEQ ID NO: 42), NNNNYYN (SEQ ID NO: 43), and NNNNYNN (SEQ ID NO: 45). In some cases, an effector protein recognizes a PAM sequence directly adjacent to a target sequence of a target nucleic acid, wherein the PAM sequence is selected from the group consisting of NNNNYYN (SEQ ID NO: 43), NNNNYTN (SEQ ID NO: 44), and NNNNYNN (SEQ ID NO: 45). In some cases, an effector protein recognizes a PAM sequence directly adjacent to a target sequence of a target nucleic acid, wherein the PAM sequence is selected from the group consisting of NNNNNYN (SEQ ID NO: 42), and NNNNYYN (SEQ ID NO: 43). In some cases, an effector protein recognizes a PAM sequence directly adjacent to a target sequence of a target nucleic acid, wherein the PAM sequence is selected from the group consisting of NNNNYYN (SEQ ID NO: 43), and NNNNYTN (SEQ ID NO: 44). In some cases, an effector protein recognizes a PAM sequence directly adjacent to a target sequence of a target nucleic acid, wherein the PAM sequence is selected from the group consisting of NNNNNYN (SEQ ID NO: 42) and NNNNYTN (SEQ ID NO: 44). In some cases, an effector protein recognizes a PAM sequence directly adjacent to a target sequence of a target nucleic acid, wherein the PAM sequence is selected from the group consisting of NNNNNYN (SEQ ID NO: 42), and NNNNYNN (SEQ ID NO: 45). In some cases, an effector protein recognizes a PAM sequence directly adjacent to a target sequence of a target nucleic acid, wherein the PAM sequence is selected from the group consisting of NNNNYYN (SEQ ID NO: 43), and NNNNYNN (SEQ ID NO: 45). In some cases, an effector protein recognizes a PAM sequence directly adjacent to a target sequence of a target nucleic acid, wherein the PAM sequence is selected from the group consisting of NNNNYNN (SEQ ID NO: 45) and NNNNYTN (SEQ ID NO: 44). In some cases, an effector protein recognizes a PAM sequence directly adjacent to a target sequence of a target nucleic acid, wherein the PAM sequence is NNNNNYN (SEQ ID NO: 42). In some cases, an effector protein recognizes a PAM sequence directly adjacent to a target sequence of a target nucleic acid, wherein the PAM sequence is NNNNYYN (SEQ ID NO: 43). In some cases, an effector protein recognizes a PAM sequence directly adjacent to a target sequence of a target nucleic acid, wherein the PAM sequence is NNNNYTN (SEQ ID NO: 44). In some cases, an effector protein recognizes a PAM sequence directly adjacent to a target sequence of a target nucleic acid, wherein the PAM sequence is NNNNYNN (SEQ ID NO: 45). In some examples, the effector protein recognizes a PAM of the sequence NYN. In some examples, the effector protein recognizes a PAM of the sequence NTT, NTC, NGT, NGC, NAT, NAC, NCC, or NCT. In some examples, the effector protein recognizes a PAM of the sequence NTT, NTC, NTG, NCT, NCC, NCG, TTT, TTC, TTG, TCT, TCC, TCG, TTTT, TTTC, TTCT, TTCC, or TTCG. In some examples, the effector protein recognizes a PAM of the sequence YYN. In some examples, the effector protein recognizes a PAM of the sequence TTN, TCN, CTN, or CCN. In some examples, the effector protein recognizes a PAM of the sequence TTA, TTG, TTC, TCA, TCG, TCC, CTA, CTG, CTC, CCA, CCG, or CCC. In some examples, the effector protein recognizes a PAM of the sequence YNN. In some examples, the effector protein recognizes a PAM of the sequence CNN or TNN. In some examples, the effector protein recognizes a PAM of the sequence CAA, CAG, CAC, CAT, CGA, CGG, CGC, CGT, CCA, CCG, CCC, CCT, CTA, CTG, CTC, CTT, TAA, TAG, TAC, TAT, TGA, TGG, TGC, TGT, TCA, TCG, TCC, TCT, TTA, TTG, TTC, or TTT.

In some cases, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM sequence directly adjacent to a target sequence of a target nucleic acid, wherein the PAM sequence is NNNNNYN (SEQ ID NO: 42). In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM sequence of NYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM sequence of NTT, NTC, NCC, or NCT. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM of the sequence ACA, ACC, ACG, ACT, ATA, ATC, ATG, ATT, CCA, CCC, CCG, CCT, CTA, CTC, CTG, CTT, GCA, GCC, GCG, GCT, GTA, GTC, GTG, GTT, TCA, TCC, TCG, TCT, TTA, TTC, TTG, or TTT. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM sequence of YYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM sequence of TTN, TCN, CTN, or CCN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 1 recognizes a PAM of the sequence TTA, TTG, TTC, TCA, TCG, TCC, CTA, CTG, CTC, CCA, CCG, or CCC.

In some cases, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM sequence directly adjacent to a target sequence of a target nucleic acid, wherein the PAM sequence is NNNNYYN (SEQ ID NO: 43). In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM sequence of YYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM of the sequence CCA, CCT, CCC, CCG, TTA, TTT, TTC, TTG, CTA, CTT, CTC, CTG, TCA, TCT, TCC, or TCG. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM sequence of NYY. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM of the sequence ACC, CCC, TCC, GCC, ACT, CCT, TCT, GCT, ATC, CTC, TTC, GTC, ATT, TTT, CTT, or GTT. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM sequence of YYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM sequence of TTN, TCN, CTN, or CCN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 2 recognizes a PAM of the sequence TTA, TTG, TTC, TCA, TCG, TCC, CTA, CTG, CTC, CCA, CCG, or CCC.

In some cases an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a PAM sequence directly adjacent to a target sequence of a target nucleic acid, wherein the PAM sequence is NNNNYTN (SEQ ID NO: 44). In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a PAM sequence of YTN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a CTA, CTC, CTT, CTG, TTA, TTC, TTT, or TTG. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a PAM sequence of NYT. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a ACT, CCT, TCT, GCT, ATT, CTT, TTT, or GTT. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a PAM sequence of YYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a PAM sequence of TTN, TCN, CTN, or CCN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 3 recognizes a PAM of the sequence TTA, TTG, TTC, TCA, TCG, TCC, CTA, CTG, CTC, CCA, CCG, or CCC.

In some cases, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 4 recognizes a PAM sequence directly adjacent to a target sequence of a target nucleic acid, wherein the PAM sequence is NNNNYNN (SEQ ID NO: 45). In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 4 recognizes a PAM sequence of NYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 4 recognizes a PAM of the sequence ACA, ACC, ACG, ACT, ATA, ATC, ATG, ATT, CCA, CCC, CCG, CCT, CTA, CTC, CTG, CTT, GCA, GCC, GCG, GCT, GTA, GTC, GTG, GTT, TCA, TCC, TCG, TCT, TTA, TTC, TTG, or TTT. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 4 recognizes a PAM sequence of YYN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 4 recognizes a PAM sequence of TTN, TCN, CTN, or CCN. In some examples, an effector protein having an amino acid sequence at least 50%, at least 55%, at least 60%, at least 65%, at least 70%, at least 75%, at least 80%, at least 85%, at least 90%, at least 95%, or 100% identical to SEQ ID NO: 4 recognizes a PAM of the sequence TTA, TTG, TTC, TCA, TCG, TCC, CTA, CTG, CTC, CCA, CCG, or CCC.

An effector protein of the present disclosure, an effector protein of the present disclosure, a dimer thereof, or a multimeric complex thereof may cleave or nick a target nucleic acid within or near PAM sequence of the target nucleic acid. In some instances, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to an engineered guide nucleic acid spacer region. As used herein for denoting protospacer adjacent motif (PAM) nucleic acid sequences, B is one or more of CG or TA; K is G or T; V is A, C or G; S is C or G; N is A, C, T, or G; Y is C or T; and R is A or G. In some instances, the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45). In some instances, the PAM sequence is NNNNNYN (SEQ ID NO: 42). In some instances, the PAM sequence is NNNNYYN (SEQ ID NO: 43). In some instances, the PAM sequence is NNNNYTN (SEQ ID NO: 44). In some instances, the PAM sequence is NNNNYNN (SEQ ID NO: 45). In some cases, the PAM sequence is 5′-TTTR-3′. In some cases, the PAM sequence is 5′-TTTN-3′ In some cases, the PAM sequence is 5′-TTTA-3′. In some cases, the PAM sequence is 5′-TTAT-3′. In some cases, the PAM sequence is 5′-TBN-3′. In some cases, the PAM sequence is 5′-TTTN-3′. In some cases, the PAM sequence is selected from the group consisting of 5′-TTTV-3′, 5′-CTTV-3′, 5′-TCTV-3′, and 5′-TTCV-3′. In some cases, the effector protein is an effector protein comprises an amino acid sequence that is at least 70%, at least 75%, at least 80%, at least 85%, at least 90%, at least 95%, or 100% identical to any one of SEQ ID NOs: 1-4, and the PAM sequence is NNNNNYN (SEQ ID NO: 42), NNNNYYN (SEQ ID NO: 43), NNNNYTN (SEQ ID NO: 44), and NNNNYNN (SEQ ID NO: 45). In some cases, the effector protein is an effector protein comprises an amino acid sequence that is at least 70%, at least 75%, at least 80%, at least 85%, at least 90%, at least 95%, or 100% identical to any one of SEQ ID NO: 1, and the PAM sequence is NNNNNYN (SEQ ID NO: 42). In some cases, the effector protein is an effector protein comprises an amino acid sequence that is at least 70%, at least 75%, at least 80%, at least 85%, at least 90%, at least 95%, or 100% identical to any one of SEQ ID NO: 2, and the PAM sequence is NNNNYYN (SEQ ID NO: 43). In some cases, the effector protein is an effector protein comprises an amino acid sequence that is at least 70%, at least 75%, at least 80%, at least 85%, at least 90%, at least 95%, or 100% identical to any one of SEQ ID NO: 3, and the PAM sequence is NNNNYTN (SEQ ID NO: 44). In some cases, the effector protein is an effector protein comprises an amino acid sequence that is at least 70%, at least 75%, at least 80%, at least 85%, at least 90%, at least 95%, or 100% identical to any one of SEQ ID NO: 4, and the PAM sequence is NNNNYNN (SEQ ID NO: 45) In some cases, the PAM sequence is 5′-TTTV-3′ (e.g., 5′-TTTG-3′). In some cases, the PAM sequence is selected from the group consisting of 5′-CTTV-3′, 5′-TCTV-3′, and 5′-TTCV-3′. In some cases, the PAM sequence. In some cases, the PAM sequence comprises 5′-VTTR-3′, such as GTTA, GTTG, ATTA, ATTG, CTTA, and CTTG. In some cases, the PAM sequence is 5′-VTTR-3′.

In some cases, the target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleosides. In some cases, the target nucleic acid comprises 10 to 90, 20 to 80, 30 to 70, or 40 to 60 linked nucleosides. A nucleic acid sequence can be from 10 to 95, from 20 to 95, from 30 to 95, from 40 to 95, from 50 to 95, from 60 to 95, from 10 to 75, from 20 to 75, from 30 to 75, from 40 to 75, from 50 to 75, from 5 to 50, from 15 to 50, from 25 to 50, from 35 to 50, or from 45 to 50 nucleotides in length. In some cases, the target nucleic acid comprises 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleosides. In some instances, the target nucleic acid comprises at least 10, at least 20, at least 30, at least 40, at least 50, at least 60, at least 70, at least 80, at least 90, or at least 100 linked nucleosides. The target nucleic acid can be reverse complementary to a guide nucleic acid. In some cases, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides of a guide nucleic acid can be reverse complementary to a target nucleic acid.

An effector protein-guide nucleic acid complex may comprise high selectivity for a target sequence. In some cases, a ribonucleoprotein may comprise a selectivity of at least 200:1, 100:1, 50:1, 20:1, 10:1, or 5:1 for a target nucleic acid over a single nucleotide variant of the target nucleic acid. In some cases, a ribonucleoprotein may comprise a selectivity of at least 5:1 for a target nucleic acid over a single nucleotide variant of the target nucleic acid. Leveraging effector protein selectivity, some methods described herein may detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample comprises 1 to 10,000, 100 to 8000, 400 to 6000, 500 to 5000, 1000 to 4000, or 2000 to 3000 target nucleic acids. In some cases, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

Often, the target nucleic acid may be from 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. The target nucleic acid may also be 0.1% to 1% of the total nucleic acids in the sample. The target nucleic acid may be DNA or RNA. The target nucleic acid may be any amount less than 100% of the total nucleic acids in the sample. The target nucleic acid may be 100% of the total nucleic acids in the sample.

The target nucleic acid may be 0.05% to 20% of total nucleic acids in the sample. Sometimes, the target nucleic acid is 0.1% to 10% of the total nucleic acids in the sample. The target nucleic acid, in some cases, is 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

A target nucleic acid may be an amplified nucleic acid of interest. The nucleic acid of interest may be any nucleic acid disclosed herein or from any sample as disclosed herein. The nucleic acid of interest may be an RNA that is reverse transcribed before amplification. The nucleic acid of interest may be amplified then the amplicons may be transcribed into RNA.

In some instances, compositions described herein exhibit indiscriminate trans-cleavage of ssRNA, enabling their use for detection of RNA in samples. In some cases, target ssRNA are generated from many nucleic acid templates (RNA) in order to achieve cleavage of the FQ reporter in the DETECTR platform. Certain effector proteins may be activated by ssRNA, upon which they may exhibit trans-cleavage of ssRNA and may, thereby, be used to cleave ssRNA FQ reporter molecules in the DETECTR system. These effector proteins may target ssRNA present in the sample, or generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described above) is capable of being cleaved by the effector protein, upon generation and amplification of ssRNA from a nucleic acid template using the methods disclosed herein, thereby generating a first detectable signal.

In some instances, target nucleic acids comprise at least one nucleic acid comprising at least 50% sequence identity to the target nucleic acid or a portion thereof. Sometimes, the at least one nucleic acid comprises an amino acid sequence that is at least 60%, 70%, 75%, 80%, 85%, 90%, 91%, 92%, 93%, 94%, 95%, 96%, 97%, 98%, or 99% identical to an equal length portion of the target nucleic acid. Sometimes, the at least one nucleic acid comprises an amino acid sequence that is 100% identical to an equal length portion of the target nucleic acid. Sometimes, the amino acid sequence of the at least one nucleic acid is at least 60%, 70%, 75%, 80%, 85%, 90%, 91%, 92%, 93%, 94%, 95%, 96%, 97%, 98%, or 99% identical to the target nucleic acid. Sometimes, the target nucleic acid comprises an amino acid sequence that is less than 60%, 70%, 75%, 80%, 85%, 90%, 91%, 92%, 93%, 94%, 95%, 96%, 97%, 98%, or 99% identical to an equal length portion of the at least one nucleic acid.

In some embodiments, samples comprise a target nucleic acid at a concentration of less than 100 μM, less than 200 μM, less than 300 μM, less than 400 μM, less than 500 μM, less than 600 μM, less than 700 μM, less than 800 μM, less than 900 μM, less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of 100 μM, to 200 μM, 200 μM to 300 μM, 300 μM to 400 μM, 400 μM to 500 μM, 500 μM to 600 μM, 600 μM to 700 μM, 700 μM to 800 μM, 800 μM to 900 μM, 900 μM to 1 nM, 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM 4 μM to 5M, 5 μM to 6 μM, 6 μM to 7M, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 200 μM to 1 nM, 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid sequence. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

A number of target nucleic acid populations are consistent with the methods and compositions disclosed herein. Some methods described herein may detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has 3 to 50, 5 to 40, or 10 to 25 target nucleic acid populations. In some cases, the method detects target nucleic acid populations that are present at least at one copy per 101 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations may be present at different concentrations or amounts in the sample.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system or an effector protein.

In some cases, the target nucleic acid is from a virus, a parasite, or a bacterium described herein. In some instances, the target nucleic acid is from a pathogen. In some instances, a pathogen is a phage, a bacterium, a virus, a parasite, a fungus, a protozoon, or a worm. In some cases, the target nucleic acid is from a bacterium. In some cases, the target nucleic acid is from a virus. In some cases, the target nucleic acid is from a parasite. In some cases, the target nucleic acid is from a fungus. In some cases, the target nucleic acid is from a protozoon. In some cases, the target nucleic acid is from a worm. In some cases, the target nucleic acid is from a phage. A pathogen may also comprise a cancer cell, a neoplastic cell, a damaged cell, a dying cell, or a foreign cell (relative to a host organism). The target nucleic acid may also be an RNA. The target nucleic acid may also be a DNA. The target nucleic acid may also be single stranded. The target nucleic acid may also be double stranded.

In some embodiments, the target nucleic acid is in a cell. In some embodiments, the cell is a single-cell eukaryotic organism; a plant cell an algal cell; a fungal cell; an animal cell; a cell an invertebrate animal; a cell a vertebrate animal such as fish, amphibian, reptile, bird, and mammal; or a cell a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

In some embodiments, the target nucleic acid comprises a nucleic acid sequence from a pathogen responsible for a disease. Non-limiting examples of pathogens are bacteria, a virus and a fungus. The target nucleic acid, in some cases, is a portion of a nucleic acid from a sexually transmitted infection or a contagious disease. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus (e.g., SARS-CoV-2); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; zika virus; norovirus; hemorrhagic fever virus; tick-borne hemorrhagic fever virus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, zika virus; rhinovirus; norovirus; hemorrhagic fever virus; tick-borne hemorrhagic fever virus; Clostridioides difficile; Helicobacter pylori; Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. Influenza virus may comprise an influenza A virus or an influenza B virus. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some instances, the target nucleic acid of a pathogen may comprise a virus. The virus may comprise a SARS-CoV-2 virus or a variant thereof, an influenza A virus or a variant thereof, an influenza B virus or a variant thereof, a human papillomavirus or a variant thereof, a herpes simplex virus or a variant thereof, an RSV or a variant thereof, a zika virus or a variant thereof, a rhinovirus or a variant thereof, a norovirus or a variant thereof, a hemorrhagic fever virus or a variant thereof, a tick-borne hemorrhagic fever virus or a variant thereof, an HIV or a variant thereof, a Hepatitis Virus C or a variant thereof, a Hepatitis Virus A or a variant thereof, a Hepatitis Virus B or a variant thereof, a herpes virus or a variant thereof, or a combination thereof. The target nucleic acid of a pathogen may comprise a SARS-CoV-2 virus or a variant thereof. The target nucleic acid of a pathogen may comprise an influenza A virus or a variant thereof. The target nucleic acid of a pathogen may comprise an influenza B virus or a variant thereof. The target nucleic acid of a pathogen may comprise a human papillomavirus or a variant thereof. The target nucleic acid of a pathogen may comprise a herpes simplex virus or a variant thereof. The target nucleic acid of a pathogen may comprise an RSV or a variant thereof. The target nucleic acid of a pathogen may comprise a zika virus or a variant thereof. The target nucleic acid of a pathogen may comprise a rhinovirus or a variant thereof. The target nucleic acid of a pathogen may comprise a norovirus or a variant thereof. The target nucleic acid of a pathogen may comprise a hemorrhagic fever virus or a variant thereof. The target nucleic acid of a pathogen may comprise a tick-borne hemorrhagic fever virus or a variant thereof. The target nucleic acid of a pathogen may comprise an HIV or a variant thereof. The target nucleic acid of a pathogen may comprise a Hepatitis Virus C. The target nucleic acid of a pathogen may comprise a Hepatitis Virus A or a variant thereof. The target nucleic acid of a pathogen may comprise a Hepatitis Virus B or a variant thereof. The target nucleic acid of a pathogen may comprise a herpes virus or a variant thereof.

In some instances, the target nucleic acid of a pathogen may comprise a bacterium. The bacterium may comprise group A Streptococcus, Chlamydia trachomatis, Neisseria gonorrhoeae, Mycoplasma genitalium, Mycobacterium tuberculosis, or a combination thereof. The target nucleic acid of a pathogen may comprise group A Streptococcus. The target nucleic acid of a pathogen may comprise Chlamydia trachomatis. The target nucleic acid of a pathogen may comprise Neisseria gonorrhoeae. The target nucleic acid of a pathogen may comprise Mycoplasma genitalium. The target nucleic acid of a pathogen may comprise Mycobacterium tuberculosis. The target nucleic acid of a pathogen may also comprise Trichomonas vaginalis. In some cases, the target nucleic acid of a pathogen comprises Candida albicans.

In some embodiments, the target nucleic acid sequence comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure (e.g., Casl4) may cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., SARS-CoV-2 (i.e., a virus that causes COVID-19), SARS, MERS, influenza, Adenovirus, Coronavirus HKU1, Coronavirus NL63, Coronavirus 229E, Coronavirus OC43, Severe Acute Respiratory Syndrome Coronavirus 2 (SARS-CoV-2), Human Metapneumovirus (hMPV), Human Rhinovirus/Enterovirus, Influenza A, Influenza A/H1, Influenza A/H3, Influenza A/H1-2009, Influenza B, Influenza C, Parainfluenza Virus 1, Parainfluenza Virus 2, Parainfluenza Virus 3, Parainfluenza Virus 4, Respiratory Syncytial Virus) and respiratory bacteria (e.g., Bordetella parapertussis, Bordetella pertussis, Chlamydia pneumoniae, Mycoplasma pneumoniae). Other viruses include human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, Chlamydia pneumoniae, Chlamydia psittaci, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g., the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g., warts (including genital, anal), herpes (including oral, genital, anal), molluscum contagiosum); hemorrhagic viral diseases (e.g., Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Bacillus anthracis, Bortadella pertussis, Burkholderia cepacia, Corynebacterium diphtheriae, Coxiella burnetii, Streptococcus agalactiae, methicillin-resistant Staphylococcus aureus, Legionella longbeachae, Legionella pneumophila, Leptospira interrogans, Moraxella catarrhalis, Streptococcus pyogenes, Escherichia coli, Neisseria gonorrhoeae, Neisseria meningitidis, Neisseria elongate, Neisseria gonorrhoeae, Parechovirus, Pneumococcus, Pneumocystis jirovecii, Cryptococcus neoformans, Histoplasma capsulatum, HAemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid may comprise a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Staphylococcus epidermidis, Legionella pneumophila, Streptococcus pyogenes, Streptococcus salivarius, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the Coronavirus HKU1 sequence is a target of an assay. In some embodiments, the Coronavirus NL63 sequence is a target of an assay. In some embodiments, the Coronavirus 229E sequence is a target of an assay. In some embodiments, the Coronavirus OC43 sequence is a target of an assay. In some embodiments, the SARS-CoV-1 sequence is a target of an assay. In some embodiments, the MERS sequence is a target of an assay. In some embodiments, the SARS-CoV-2 sequence is a target of an assay. In some embodiments, the Respiratory Syncytial Virus A sequence is a target of an assay. In some embodiments, the Respiratory Syncytial Virus B sequence is a target of an assay. In some embodiments, the Influenza A sequence is a target of an assay. In some embodiments, the Influenza B sequence is a target of an assay. In some embodiments, the Human Metapneumovirus sequence is a target of an assay. In some embodiments, the Human Rhinovirus sequence is a target of an assay. In some embodiments, the Human Enterovirus sequence is a target of an assay. In some embodiments, the Parainfluenza Virus 1 sequence is a target of an assay. In some embodiments, the Parainfluenza Virus 2 sequence is a target of an assay. In some embodiments, the Parainfluenza Virus 3 sequence is a target of an assay. In some embodiments, the Parainfluenza Virus 4 sequence is a target of an assay. In some embodiments, the Alphacoronavirus genus sequence is a target of an assay. In some embodiments, the Betacoronavirus genus sequence is a target of an assay. In some embodiments, the Sarbecovirus subgenus sequence is a target of an assay. In some embodiments, the SARS-related virus species sequence is a target of an assay. In some embodiments, the Gammacoronavirus Genus sequence is a target of an assay. In some embodiments, the Deltacoronavirus Genus sequence is a target of an assay. In some embodiments, the Influenza B—Victoria V1 sequence is a target of an assay. In some embodiments, the Influenza B—Yamagata Y1 sequence is a target of an assay. In some embodiments, the Influenza A H1 sequence is a target of an assay. In some embodiments, the Influenza A H2 sequence is a target of an assay. In some embodiments, the Influenza A H3 sequence is a target of an assay. In some embodiments, the Influenza A H4 sequence is a target of an assay. In some embodiments, the Influenza A H5 sequence is a target of an assay. In some embodiments, the Influenza A H6 sequence is a target of an assay. In some embodiments, the Influenza A H7 sequence is a target of an assay. In some embodiments, the Influenza A H8 sequence is a target of an assay. In some embodiments, the Influenza A H9 sequence is a target of an assay. In some embodiments, the Influenza A H10 sequence is a target of an assay. In some embodiments, the Influenza A H11 sequence is a target of an assay. In some embodiments, the Influenza A H12 sequence is a target of an assay. In some embodiments, the Influenza A H13 sequence is a target of an assay. In some embodiments, the Influenza A H14 sequence is a target of an assay. In some embodiments, the Influenza A H15 sequence is a target of an assay. In some embodiments, the Influenza A H16 sequence is a target of an assay. In some embodiments, the Influenza A N1 sequence is a target of an assay. In some embodiments, the Influenza A N2 sequence is a target of an assay. In some embodiments, the Influenza A N3 sequence is a target of an assay. In some embodiments, the Influenza A N4 sequence is a target of an assay. In some embodiments, the Influenza A N5 sequence is a target of an assay. In some embodiments, the Influenza A N6 sequence is a target of an assay. In some embodiments, the Influenza A N7 sequence is a target of an assay. In some embodiments, the Influenza A N8 sequence is a target of an assay. In some embodiments, the Influenza A N9 sequence is a target of an assay. In some embodiments, the Influenza A N10 sequence is a target of an assay. In some embodiments, the Influenza A N11 sequence is a target of an assay. In some embodiments, the Influenza A/H1-2009 sequence is a target of an assay. In some embodiments, the Human endogenous control 18S rRNA sequence is a target of an assay. In some embodiments, the Human endogenous control GAPDH sequence is a target of an assay. In some embodiments, the Human endogenous control HPRT1 sequence is a target of an assay. In some embodiments, the Human endogenous control GUSB sequence is a target of an assay. In some embodiments, the Human endogenous control RNASe P sequence is a target of an assay. In some embodiments, the Influenza A oseltamivir resistance sequence is a target of an assay. In some embodiments, the Human Bocavirus sequence is a target of an assay. In some embodiments, the SARS-CoV-2 85Δ sequence is a target of an assay. In some embodiments, the SARS-CoV-2 T1001I sequence is a target of an assay. In some embodiments, the SARS-CoV-2 3675-3677Δ sequence is a target of an assay. In some embodiments, the SARS-CoV-2 P4715L sequence is a target of an assay. In some embodiments, the SARS-CoV-2 S5360L sequence is a target of an assay. In some embodiments, the SARS-CoV-2 69-70Δ sequence is a target of an assay. In some embodiments, the SARS-CoV-2 Tyrl44fs sequence is a target of an assay. In some embodiments, the SARS-CoV-2 242-244Δ sequence is a target of an assay. In some embodiments, the SARS-CoV-2 Y453F sequence is a target of an assay. In some embodiments, the SARS-CoV-2 S477N sequence is a target of an assay. In some embodiments, the SARS-CoV-2 E848K sequence is a target of an assay. In some embodiments, the SARS-CoV-2 N501Y sequence is a target of an assay. In some embodiments, the SARS-CoV-2 D614G sequence is a target of an assay. In some embodiments, the SARS-CoV-2 P681R sequence is a target of an assay. In some embodiments, the SARS-CoV-2 P681H sequence is a target of an assay. In some embodiments, the SARS-CoV-2 L21F sequence is a target of an assay. In some embodiments, the SARS-CoV-2 Q27Stop sequence is a target of an assay. In some embodiments, the SARS-CoV-2 M1fs sequence is a target of an assay. In some embodiments, the SARS-CoV-2 R203fs sequence is a target of an assay. In some embodiments, the Human adenovirus—pan assay sequence is a target of an assay. In some embodiments, the Bordetella parapertussis sequence is a target of an assay. In some embodiments, the Bordetella pertussis sequence is a target of an assay. In some embodiments, the Chlamydophila pneumoniae sequence is a target of an assay. In some embodiments, the Mycoplasma pneumoniae sequence is a target of an assay. In some embodiments, the Legionella pneumophila sequence is a target of an assay. In some embodiments, the Bordetella bronchoseptica sequence is a target of an assay. In some embodiments, the Bordetella holmesii sequence is a target of an assay. In some embodiments, the Human adenovirus Type A sequence is a target of an assay. In some embodiments, the Human adenovirus Type B sequence is a target of an assay. In some embodiments, the Human adenovirus Type C sequence is a target of an assay. In some embodiments, the Human adenovirus Type D sequence is a target of an assay. In some embodiments, the Human adenovirus Type E sequence is a target of an assay. In some embodiments, the Human adenovirus Type F sequence is a target of an assay. In some embodiments, the Human adenovirus Type G sequence is a target of an assay. In some embodiments, the MERS-CoV sequence is a target of an assay. In some embodiments, the human metapneumovirus sequence is a target of an assay. In some embodiments, the human parainfluenza 1 sequence is a target of an assay. In some embodiments, the human parainfluenza 2 sequence is a target of an assay. In some embodiments, the human parainfluenza 4 sequence is a target of an assay. In some embodiments, the hCoV-OC43 sequence is a target of an assay. In some embodiments, the human parainfluenza 3 sequence is a target of an assay. In some embodiments, the RSV-A sequence is a target of an assay. In some embodiments, the RSV-B sequence is a target of an assay. In some embodiments, the hCoV-229E sequence is a target of an assay. In some embodiments, the hCoV-HKU1 sequence is a target of an assay. In some embodiments, the hCoV-NL63 sequence is a target of an assay. In some embodiments, the Gammacoronavirus sequence is a target of an assay. In some embodiments, the Deltacoronavirus sequence is a target of an assay. In some embodiments, the Alphacoronavirus sequence is a target of an assay. In some embodiments, the Rhinovirus C sequence is a target of an assay. In some embodiments, the Betacoronavirus sequence is a target of an assay. In some embodiments, the Influenza A sequence is a target of an assay. In some embodiments, the Influenza B sequence is a target of an assay. In some embodiments, the SARS-CoV-2 sequence is a target of an assay. In some embodiments, the SARS-CoV-1 sequence is a target of an assay. In some embodiments, the Sarbecovirus subgenus sequence is a target of an assay. In some embodiments, the SARS-related viruses sequence is a target of an assay. In some embodiments, the MS2 sequence is a target of an assay.

In some embodiments, the assay is directed to one or more target sequences. In some embodiments, a target sequence is a portion of an antimicrobial resistance (AMR) gene, such as CTX-M-1, CTX-M-2, CTX-M-25, CTX-M-8, CTX-M-9, or IMP. In some embodiments, a target sequence is a Mycobacterium tuberculosis sequence, such as a portion of IS1081 or IS6110. In some embodiments, a target sequence is an orthopox virus sequence. In some embodiments, a target sequence is a pseudorabies virus sequence. In some embodiments, a target sequence is a Staphylococcus aureus sequence, such as a portion of gyrA or gyrB, or a portion of a S. aureus thermonuclease. In some embodiments, a target sequence is a Stenotrophomonas maltophilia sequence, such as a sequence of S. maltophilia alpha, S. maltophilia beta, or S. maltophilia gamma. In some embodiments, a target sequence is a Bordetalla sp. sequence, such as a sequence of Bordetella bronchoseptica, Bordetella holmesii, Bordetella parapertussis, or Bordetella pertussis. In some embodiments, a target sequence is a Chlamydophila pneumoniae sequence. In some embodiments, a target sequence is a Human adenovirus sequence, such as a sequence of human adenovirus Type A, Type B, Type C, Type D, Type E, Type F, or Type G. In some embodiments, a target sequence is a human bocavirus sequence. In some embodiments, a target sequence is a Legionella pneumophila sequence. In some embodiments, a target sequence is a Mycoplasma pneumoniae sequence. In some embodiments, a target sequence is an Acinetobacter spp. (e.g., A. pitii, A. baumannii, or A. nosocomialis) sequence, such as a portion of gyrB or a 16S-23S ribosomal RNA intergenic spacer sequence. In some embodiments, a target sequence is a Proteus spp. (e.g. P. mirabilis, P. vulgaris, P. penneri, or P. hauseri) sequence, such as a portion of rpoD or 16S. In some embodiments, a target sequence is an Enterobacter spp. (e.g. E. nimipressuralis, E. cloacae, E. asburiae, E. hormaechei, E. kobei, E. ludwigii, or E. mori) sequence, such as a portion of dnaJ, purG, or 165. In some embodiments, a target sequence is a Bacillus anthracis sequence, such as a portion of pagA or capB. In some embodiments, a target sequence is a Brucella spp. sequence, such as a portion of 23S, bcsp3i, or omp2a. In some embodiments, a target sequence is a Coxiella burnetiid sequence, such as a portion of com1 or IS110. In some embodiments, a target sequence is a Francisella tularensis sequence, such as a portion of 165. In some embodiments, a target sequence is a Rickettsia spp. sequence, such as a portion of 165, 23S, or 782-17K genus common antigen. In some embodiments, a target sequence is a Yersinia pestis sequence, such as a portion of pMT1, pCD1, or pPCP1. In some embodiments, a target sequence is a A. calcoaceticus sequence, such as a portion of gyrB. In some embodiments, a target sequence is a Francisella tularensis sequence, such as a portion of tul4 or fopA. In some embodiments, a target sequence is an rRNA sequence, such as a portion of 28S rRNA or 18S rRNA. In some embodiments, a target sequence is a coronavirus sequence, such as a sequence of an alphacoronavirus, betacoronavirus, deltacoronavirus, or gammacoronavirus. In some embodiments, a target sequence is a human coronavirus (hCoV) sequence, such as a sequence of hCoV-229E, hCoV-HKU1, hCoV-NL63, hCoV-OC43. In some embodiments, a target sequence is a MERS-CoV sequence. In some embodiments, the sequence is a mammarenavirus sequence, such as a sequence of a Argentinian mammarenavirus (Junin arenavirus), Lassa mammarenavirus, Lujo mammarenavirus (e.g., an L segment or S segment thereof), or Machupo mammarenavirus. In some embodiments, a target sequence is a human metapneumovirus sequence. In some embodiments, a target sequence is a human parainfluenza sequence, such as a sequence of human parainfluenza 1, human parainfluenza 2, human parainfluenza 3, or human parainfluenza 4. In some embodiments, a target sequence is an influenza A virus sequence, such as a sequence of influenza A H1, H2, H3, H4, H5, H6, H7, H8, H9, H10, H11, H12, H13, H14, H15, H16, N1, N2, N3, N4, N5, N6, N7, N8, or N9. In some embodiments, a target sequence is an influenza B sequence, such as a sequence of influenza B-Victoria V1 or influenza B-Yamagata Y1. In some embodiments, a target sequence is a bacteriophage MS2 sequence. In some embodiments, a target sequence is a rhinovirus C sequence. In some embodiments, a target sequence is a respiratory syncytial virus (RSV) sequence, such as a sequence of RSV-A or RSV-B. In some embodiments, a target sequence is a Sarbecovirus sequence. In some embodiments, a target sequence is a severe acute respiratory syndrome coronavirus (SARS-CoV) sequence, such as a sequence of SARS-CoV-1 or SARS-CoV-2. In some embodiments, a target sequence is a portion of a SARS-COV-2 S gene, such as a sequence comprising 144/145 wild-type (WT), deletion (del) 144/145 (alpha variant), 156/157 WT, deli56/157 (delta variant), 241/243 WT, del241/243 (beta variant), 69/70 WT, de169/70 (alpha variant), A570 WT, A570D (alpha variant), A701 WT, A701V (beta variant), D1118 WT, D1118H (alpha variant), D215 WT, D215G (beta variant), D614 WT, D614G (beta variant), D80 WT, D80A (beta variant), E484 WT, E484K (gamma variant), P681 WT, P681H (alpha variant), P681R (delta variant), S982 WT, S982A (alpha variant), T19 WT, T19R (delta variant), T716 WT, T716F (gamma variant). In some embodiments, a target sequence is a SARS-related virus sequence. In some embodiments, a target sequence is a portion of a gene selected from 165, 23S, ACTB, ATP5ME, ATP5MF, ATP5MG, ATP5PB, BCSP31, CAPB, CHMP2A, Clorf43, COM1, DNAJ, EMC7, FOPA, GPI, GAPDH, GUSB, GYRB, HRPT1, NDUFB3, NDUFB4, NDUFB8, OMP2A, PAGA, PRDX1, PSMB2, PSMB4, PURG, RAB7A, REEP5, RNaseP, RPL13, RPL19, RPL27A, RPL30, RPL31, RPL32, RPL37A, RPOD, RPS10, RPS27, RPS29, RPS6, SNRPD3, TUL4, VCP, VPS29, and YWHAG.

In some embodiments, the one or more targets may be at a concentration of 1 copy/reaction, at least about 2 copies/reaction, at least about 3 copies/reaction, at least about 4 copies/reaction, at least about 5 copies/reaction, at least about 6 copies/reaction, at least about 7 copies/reaction, at least about 8 copies/reaction, at least about 9 copies/reaction, at least about 10 copies/reaction, at least about 20 copies/reaction, at least about 30 copies/reaction, at least about 40 copies/reaction, at least about 50 copies/reaction, at least about 60 copies/reaction, at least about 70 copies/reaction, at least about 80 copies/reaction, at least about 90 copies/reaction, at least about 100 copies/reaction, at least about 200 copies/reaction, at least about 300 copies/reaction, at least about 400 copies/reaction, at least about 500 copies/reaction, at least about 600 copies/reaction, at least about 700 copies/reaction, at least about 800 copies/reaction, at least about 900 copies/reaction, at least about 1000 copies/reaction, at least about 2000 copies/reaction, at least about 3000 copies/reaction, at least about 4000 copies/reaction, at least about 5000 copies/reaction, at least about 6000 copies/reaction, at least about 7000 copies/reaction, at least about 8000 copies/reaction, at least about 9000 copies/reaction, at least about 10000 copies/reaction, at least about 20000 copies/reaction, at least about 30000 copies/reaction, at least about 40000 copies/reaction, at least about 50000 copies/reaction, at least about 60000 copies/reaction, at least about 70000 copies/reaction, at least about 80000 copies/reaction, at least about 90000 copies/reaction, or at least about 100000 copies/reaction.

### Mutations

In some instances, target nucleic acids comprise a mutation. In some instances, a sequence comprising a mutation may be modified to a wildtype sequence with a composition, system or method described herein. In some instances, a sequence comprising a mutation may be detected with a composition, system or method described herein. The mutation may be a mutation of 1, 2, 3, 4, 5, 6, 7, 8, 9, 10, 11, 12, 13, 14, 15, 16, 17, 18, 19, 20 or more nucleotides. Non-limiting examples of mutations are insertion-deletion (indel), single nucleotide polymorphism (SNP), and frameshift mutations. In some instances, guide nucleic acids described herein hybridize to a region of the target nucleic acid comprising the mutation. The may be located in a non-coding region or a coding region of a gene.

In some instances, target nucleic acids comprise a mutation, wherein the mutation is a SNP. The single nucleotide mutation or SNP may be associated with a phenotype of the sample or a phenotype of the organism from which the sample was taken. The SNP, in some cases, is associated with altered phenotype from wild type phenotype. The SNP may be a synonymous substitution or a nonsynonymous substitution. The nonsynonymous substitution may be a missense substitution or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a maycer cell.

In some instances, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some instances, the target nucleic acid comprises a mutation associated with a disease. In some examples, a mutation associated with a disease refers to a mutation whose presence in a subject indicates that the subject is susceptible to, or suffers from, a disease, disorder, or pathological state. In some examples, a mutation associated with a disease refers to a mutation which causes the disease, contributes to the development of the disease, or indicates the existence of the disease. A mutation associated with a disease may also refer to any mutation which generates transcription or translation products at an abnormal level, or in an abnormal form, in cells affected by a disease relative to a control without the disease.

The mutation may cause the disease. The disease may comprise, at least in part, a cancer, an inherited disorder, an ophthalmological disorder, a neurological disorder, a blood disorder, a metabolic disorder, or a combination thereof. The disease may comprise, at least in part, a cancer. The disease may comprise, at least in part, an inherited disorder. The disease may comprise, at least in part, an ophthalmological disorder. The disease may comprise, at least in part, a neurological disorder. The disease may comprise, at least in part, a blood disorder. The disease may comprise, at least in part, a metabolic disorder. In some instances, the target nucleic acid comprises a mutation associated with a disease. The mutation may cause the disease. The disease may comprise an inherited disorder, an ophthalmological disorder, a neurological disorder, a blood disorder, a metabolic disorder, or a combination thereof. The disease may comprise, at least in part, a cancer. The disease may comprise, at least in part, an inherited disorder. The disease may comprise, at least in part, an ophthalmological disorder. The disease may comprise, at least in part, a neurological disorder. The disease may comprise, at least in part, a blood disorder. The disease may comprise, at least in part, a metabolic disorder.

In some cases, the neurological disorder comprises Duchenne muscular dystrophy, myotonic dystrophy Type 1, or cystic fibrosis. In some cases, the neurological disorder comprises Duchenne muscular dystrophy. In some cases, the neurological disorder comprises myotonic dystrophy Type 1. In some cases, the neurological disorder comprises cystic fibrosis. In some cases, the neurological disorder comprises a neurodegenerative disease.

### XIII. Certain Samples

The systems and methods of the present disclosure can be used to detect one or more target sequences or nucleic acids in one or more samples. Various sample types comprising a target nucleic acid of interest are consistent with the present disclosure. These samples may comprise a target nucleic acid sequence for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest.

In some instances, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, any combination thereof. A tissue sample from a subject may be dissociated or liquified prior to application to detection system of the present disclosure. A sample can be an aspirate of a bodily fluid from an animal (e.g., human, animals, livestock, pet, etc.) or plant. A tissue sample can be from any tissue that can be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). Non-limiting examples of environmental samples are soil, air, or water. In some instances, an environmental sample is taken as a swab from a surface of interest or taken directly from the surface of interest.

In some instances, the sample is a raw (unprocessed, unmodified) sample. Raw samples may be applied to a system for detecting or modifying a target nucleic acid, such as those described herein. In some instances, the sample is diluted with a buffer or a fluid or concentrated prior to its application to the system or be applied neat to the detection system. In some cases, the sample is contained in no more than about 200 nanoliters (nL). In some cases, the sample is contained in about 200 nL. In some cases, the sample is contained in a volume that is greater than about 200 nL and less than about 20 microliters (μL). Sometimes, the sample contains no more 20 μl of buffer or fluid. The sample, in some cases, is contained in no more than 1, 5, 10, 15, 20, 25, 30, 35 40, 45, 50, 55, 60, 65, 70, 75, 80, 90, 100, 200, 300, 400, 500 μl. In some cases, the sample is contained in from 1 μL to 500 μL, from 10 μL to 500 μL, from 50 μL to 500 μL, from 100 μL to 500 μL, from 200 μL to 500 μL, from 300 μL to 500 μL, from 400 μL to 500 μL, from 1 μL to 200 μL, from 10 μL to 200 μL, from 50 μL to 200 μL, from 100 μL to 200 μL, from 1 μL to 100 μL, from 10 μL to 100 μL, from 50 μL to 100 μL, from 1 μL to 50 μL, from 10 μL to 50 μL, from 1 μL to 20 μL, from 10 μL to 20 μL, or from 1 μL to 10 μL, or any of value 1 μl to 500 μl, preferably 10 μL to 200 μL, or more preferably 50 μL to 100 μL of buffer or fluid. Sometimes, the sample is contained in more than 500 μl.

In some instances, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

In some instances, samples are used for diagnosing a disease. In some instances, the disease is cancer. The sample used for cancer testing may comprise at least one target nucleic acid that may bind to an engineered guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay may be used to detect “hotspots” in target nucleic acids that may be predictive of lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid comprises a portion of a nucleic acid that is associated with a blood fever.

In some embodiments, the target nucleic acid comprises a portion or a specific region of a nucleic acid from a genomic locus, any DNA amplicon of, a reverse transcribed mRNA, or a cDNA from one or more genes selected from AAVS1, ABCA4, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AHI1, AIRE, ALDH3A2, ALDOB, ALG6, ALK, ALKBH5, ALMS1, ALPL, AMRC9, AMT, ANGPTL3, APC, Apo(a), APOCIII, APOEE4, APOL1, APP, AQP2, AR, ARFRP1, ARG1, ARL13B, ARL6, ARSA, ARSB, ASL, ASNS, ASPA, ASS1, ATM, ATP6V1B1, ATP7A, ATP7B, ATRX, ATXN1, ATXN10, ATXN2, ATXN3, ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAFV600E, BRCA1, BRCA2, BRIP1, BSND, C282Y, C9orf72, CA4, CACNA1A, CAPN3, CASR, CBS, CC2D2A, CCR5, CDC73, CDH1, CDH23, CDK11, CDK4, CDKN1B, CDKN1C, CDKN2A, CEBPA, CEP290, CERKL, CFTR, CHCHD10, CHEK2, CHM, CHRNE, CIITA, CLN3, CLN5, CLN6, CLN8, CLRN1, CLTA, CNBP, CNGB1, CNGB3, COL1A1, COL1A2, COL27A1, COL4A3, COL4A4, COL4A5, COL7A1, CPS1, CPT1A, CPT2, CRB1, CRX, CTNNA1, CTNNB1, CTNND2, CTNS, CTSK, CYBA, CYBB, CYP11B1, CYP11B2, CYP17A1, CYP19A1, CYP27A1, DBT, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DYSF, EDA, EDN3, EDNRB, EGFR, EIF2B5, EMC2, EMC3, EMD, EMX1, EPCAM, ERCC6, ERCC8, ESCO2, ETFA, ETFDH, ETHEl, EVC, EVC2, EYS, F5, F9, FactorB, FactorXI, FAH, FAM161A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FH, FHL1, FKRP, FKTN, FLCN, FMR1, FOXP3, FSCN2, FUS, FUT8, FVIII, FXII, FXN, G6PC, GAA, GALC, GALK1, GALT, GAMT, GATA2, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPC3, GPR98, GREM1, GRHPR, GRIN2B, H2AX, HADHA, HAX1, HBA1, HBA2, HBB, HEXA, HEXB, HGSNAT, HLCS, HMGCL, HOGA1, HOXB13, HPRPF3, HPRT1, HPS1, HPS3, HRAS, HSD17B4, HSD3B2, HTT, HYAL1, HYLS1, IDS, IDUA, IFITM5, IKBKAP, IL2RG, IMPDH1, INPP5E, IRF4, ITPR1, IVD, JAG1, KCNC3, KCND3, KCNJ11, KLHL7, KRAS, LAMA2, LAMA3, LAMB3, LAMC2, LCA5, LDLR, LDLRAP1, LHX3, LIFR, LIPA, LMNA, LOXHD1, LPL, LRAT, LRP6, LRPPRC, LRRK2, MAN2B1, MAPT, MAX, MCOLN1, MECP2, MED17, MEFV, MEN1, MERTK, MESP2, MET, METex14, MFN2, MFSD8, MITF, MKS1, MLC1, MLH1, MLH3, MMAA, MMAB, MMACHC, MMADHC, MMD, MPI, MPL, MPV17, MSH2, MSH3, MSH6, MTHFR, MTM1, MTRR, MTTP, MUT, MUTYH, MYO7A, NAGLU, NAGS, NBN, NDRG1, NDUFAF5, NDUFS6, NEB, NF1, NF2, NOTCH2, NPC1, NPC2, NPHP1, NPHS1, NPHS2, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAR1A, PRKCG, PRNP, PROM1, PROP1, PRPF31, PRPF8, PRPH2, PRPS1, PSAP, PSD95, PSEN1, PSEN2, PTCH1, PTEN, PTS, PUS1, PYGM, RAB23, RAD50, RAD51C, RAD51D, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTEL1, RUNX1, SACS, SAMHD1, SCN1A, SCN2A, SDHA, SDHAF2, SDHB, SDHC, SDHD, SEL1L, SEPSECS, SERPINGI, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMAD4, SMARCA4, SMARCAL1, SMARCB1, SMARCEl, SMN1, SMPD1, SNAI2, SNCA, SNRNP200, SOD1, SOX10, SPARA7, SPTBN2, STAR, STAT3, STK11, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WDR19, WNT10A, WS2B, WS2C, XPA, XPC, XPF, YAP1, ZFYVE26, and ZNF423. Further description of editing or detecting a target nucleic acid in the foregoing genes can be found in more detail in Kim et al., “Enhancement of target specificity of CRISPR-Cas12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus a-hemolysin-mediated toxicity”, Scientific Reports volume 6, Article number: 24242 (2016); and Ma et al., “A CRISPR-Based Screen Identifies Genes Essentialfor West-Nile-Virus-Induced Cell Death”, Cell Rep. 2015 Jul. 28; 12(4):673-83, which are hereby incorporated by reference in their entirety. The target nucleic acids described herein can be used as target nucleic acids for methods for editing a target nucleic acid described above.

In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCEl, STK11, SUFU, TERC, TERT, TMEM127, TP53, TSC1, TSC2, VHL, WRN, and WT1. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some cases, the target nucleic acid is encoded by a gene selected from TABLE 4. In some cases, the target nucleic acid comprises a portion of a nucleic acid from a genomic locus, any DNA amplicon of, a reverse transcribed mRNA, or a cDNA from a locus selected from TABLE 4.

TABLE 4
Exemplary target nucleic acids

DNMT1, HPRT1, RPL32P3, CCR5, FANCF, GRIN2B, EMX1
AAVS1, ALKBH5, CLTA, CDK11
CTNNB1, AXIN1, LRP6, TBK1, BAP1, TLE3, PPMIA, BCL2L2,
SUFU, RICTOR, VPS35, TOP1, SIRT1, PTEN
MMD, PAQR8
H2AX, POU5F1, OCT4
SYS1, ARFRP1, TSPAN14
EMC2, EMC3, SELIL, DERL2, UBE2G2, UBE2J1, HRD1

In some cases, the target nucleic acid is encoded by a gene selected from DNMT1, HPRT1, RPL32P3, CCR5, FANCF, GRIN2B, EMX1, AAVS1, ALKBH5, CLTA, CDK11, CTNNB1, AXIN1, LRP6, TBK1, BAP1, TLE3, PPM1A, BCL2L2, SUFU, RICTOR, VPS35, TOP1, SIRT1, PTEN, MMD, PAQR8, H2AX, POU5F1, OCT4, SYS1, ARFRP1, TSPAN14, EMC2, EMC3, SEL1L, DERL2, UBE2G2, UBE2J1, and HRD1.

In some cases, the gene is PCSK9. In some cases, the gene is TRAC, B2M, PD1, or a combination thereof. In some cases, the contacting occurs in vitro. In some cases, the contacting occurs in vivo. In some cases, the contacting occurs ex vivo.

In some cases, the target nucleic acid comprises a portion of a nucleic acid from a genomic locus, any DNA amplicon of, a reverse transcribed mRNA, or a cDNA from a locus of at least one of: DNMT1, HPRT1, RPL32P3, CCR5, FANCF, GRIN2B, and EMX1. In some cases, the target nucleic acid comprises a portion of a nucleic acid from a genomic locus, any DNA amplicon of, a reverse transcribed mRNA, or a cDNA from DNMT1. In some cases, the target nucleic acid comprises a portion of a nucleic acid from a genomic locus, any DNA amplicon of, a reverse transcribed mRNA, or a cDNA from HPRT1. In some cases, the target nucleic acid comprises a portion of a nucleic acid from a genomic locus, any DNA amplicon of, a reverse transcribed mRNA, or a cDNA from RPL32P3. In some cases, the target nucleic acid comprises a portion of a nucleic acid from a genomic locus, any DNA amplicon of, a reverse transcribed mRNA, or a cDNA from CCR5. In some cases, the target nucleic acid comprises a portion of a nucleic acid from a genomic locus, any DNA amplicon of, a reverse transcribed mRNA, or a cDNA from FANCF. In some cases, the target nucleic acid comprises a portion of a nucleic acid from a genomic locus, any DNA amplicon of, a reverse transcribed mRNA, or a cDNA from GRIN2B. In some cases, the target nucleic acid comprises a portion of a nucleic acid from a genomic locus, any DNA amplicon of, a reverse transcribed mRNA, or a cDNA from EMX1. DNMT1, HPRT1, RPL32P3, CCR5, FANCF, GRIN2B, or EMX1 has been described in more detail in Kim et al., “Enhancement of target specificity of CRISPR-Casl2a by using a chimeric DNA-RNA guide”, Nucleic Acids Res. 2020 Sep. 4; 48(15):8601-8616, which is hereby incorporated by reference in its entirety.

In some cases, the target nucleic acid comprises a portion of a nucleic acid from a genomic locus, any DNA amplicon of, a reverse transcribed mRNA, or a cDNA from a locus of at least one of: AAVS1, ALKBH5, CLTA, and CDK11. In some cases, the target nucleic acid comprises a portion of a nucleic acid from a genomic locus, any DNA amplicon of, a reverse transcribed mRNA, or a cDNA from AAVS1. In some cases, the target nucleic acid comprises a portion of a nucleic acid from a genomic locus, any DNA amplicon of, a reverse transcribed mRNA, or a cDNA from ALKBH5. In some cases, the target nucleic acid comprises a portion of a nucleic acid from a genomic locus, any DNA amplicon of, a reverse transcribed mRNA, or a cDNA from CLTA. In some cases, the target nucleic acid comprises a portion of a nucleic acid from a genomic locus, any DNA amplicon of, a reverse transcribed mRNA, or a cDNA from CDK11. AAVS1, ALKBH5, CLTA, or CDK11 has been described in more detail in Wang et al., “Specificity profiling of CRISPR system reveals greatly enhanced off-target gene editing”, Scientific Reports volume 10, Article number: 2269 (2020), which is hereby incorporated by reference in its entirety.

In some cases, the target nucleic acid comprises a portion of a nucleic acid from a genomic locus, any DNA amplicon of, a reverse transcribed mRNA, or a cDNA from a locus of at least one of: CTNNB1, AXIN1, LRP6, TBK1, BAP1, TLE3, PPM1A, BCL2L2, SUFU, RICTOR, VPS35, TOP1, SIRT1, and PTEN. In some cases, the target nucleic acid comprises a portion of a nucleic acid from a genomic locus, any DNA amplicon of, a reverse transcribed mRNA, or a cDNA from CTNNB1. In some cases, the target nucleic acid comprises a portion of a nucleic acid from a genomic locus, any DNA amplicon of, a reverse transcribed mRNA, or a cDNA from AXIN1. In some cases, the target nucleic acid comprises a portion of a nucleic acid from a genomic locus, any DNA amplicon of, a reverse transcribed mRNA, or a cDNA from LRP6. In some cases, the target nucleic acid comprises a portion of a nucleic acid from a genomic locus, any DNA amplicon of, a reverse transcribed mRNA, or a cDNA from TBK1. In some cases, the target nucleic acid comprises a portion of a nucleic acid from a genomic locus, any DNA amplicon of, a reverse transcribed mRNA, or a cDNA from BAP1. In some cases, the target nucleic acid comprises a portion of a nucleic acid from a genomic locus, any DNA amplicon of, a reverse transcribed mRNA, or a cDNA from TLE3. In some cases, the target nucleic acid comprises a portion of a nucleic acid from a genomic locus, any DNA amplicon of, a reverse transcribed mRNA, or a cDNA from PPM1A. In some cases, the target nucleic acid comprises a portion of a nucleic acid from a genomic locus, any DNA amplicon of, a reverse transcribed mRNA, or a cDNA from BCL2L2. In some cases, the target nucleic acid comprises a portion of a nucleic acid from a genomic locus, any DNA amplicon of, a reverse transcribed mRNA, or a cDNA from SUFU. In some cases, the target nucleic acid comprises a portion of a nucleic acid from a genomic locus, any DNA amplicon of, a reverse transcribed mRNA, or a cDNA from RICTOR. In some cases, the target nucleic acid comprises a portion of a nucleic acid from a genomic locus, any DNA amplicon of, a reverse transcribed mRNA, or a cDNA from VPS35. In some cases, the target nucleic acid comprises a portion of a nucleic acid from a genomic locus, any DNA amplicon of, a reverse transcribed mRNA, or a cDNA from TOP1. In some cases, the target nucleic acid comprises a portion of a nucleic acid from a genomic locus, any DNA amplicon of, a reverse transcribed mRNA, or a cDNA from SIRT1. In some cases, the target nucleic acid comprises a portion of a nucleic acid from a genomic locus, any DNA amplicon of, a reverse transcribed mRNA, or a cDNA from PTEN. CTNNB1, AXIN1, LRP6, TBK1, BAP1, TLE3, PPM1A, BCL2L2, SUFU, RICTOR, VPS35, TOP1, SIRT1, or PTEN has been described in more detail in Tuladhar et al., “CRISPR-Cas9-based mutagenesis frequently provokes on-target mRNA misregulation”, Nature Communications volume 10, Article number: 4056 (2019), which is hereby incorporated by reference in its entirety.

In some cases, the target nucleic acid comprises a portion of a nucleic acid from a genomic locus, any DNA amplicon of, a reverse transcribed mRNA, or a cDNA from a locus of at least one of: MMD and PAQR8. In some cases, the target nucleic acid comprises a portion of a nucleic acid from a genomic locus, any DNA amplicon of, a reverse transcribed mRNA, or a cDNA from MMD. In some cases, the target nucleic acid comprises a portion of a nucleic acid from a genomic locus, any DNA amplicon of, a reverse transcribed mRNA, or a cDNA from PAQR8. MMD or PAQR8 has been described in more detail in Dong et al., “Genome-Wide Off-Target Analysis in CRISPR-Cas9 Modified Mice and Their Offspring”, G3, Volume 9, Issue 11, 1 Nov. 2019, Pages 3645-3651, which is hereby incorporated by reference in its entirety.

In some cases, the target nucleic acid comprises a portion of a nucleic acid from a genomic locus, any DNA amplicon of, a reverse transcribed mRNA, or a cDNA from a locus of at least one of: H2AX, POU5F1, and OCT4. In some cases, the target nucleic acid comprises a portion of a nucleic acid from a genomic locus, any DNA amplicon of, a reverse transcribed mRNA, or a cDNA from H2AX. In some cases, the target nucleic acid comprises a portion of a nucleic acid from a genomic locus, any DNA amplicon of, a reverse transcribed mRNA, or a cDNA from POU5F1. In some cases, the target nucleic acid comprises a portion of a nucleic acid from a genomic locus, any DNA amplicon of, a reverse transcribed mRNA, or a cDNA from OCT4.

In some cases, the target nucleic acid comprises a portion of a nucleic acid from a genomic locus, any DNA amplicon of, a reverse transcribed mRNA, or a cDNA from a locus of at least one of: SYS1, ARFRP1, and TSPAN14. In some cases, the target nucleic acid comprises a portion of a nucleic acid from a genomic locus, any DNA amplicon of, a reverse transcribed mRNA, or a cDNA from SYS1. In some cases, the target nucleic acid comprises a portion of a nucleic acid from a genomic locus, any DNA amplicon of, a reverse transcribed mRNA, or a cDNA from ARFRP1. In some cases, the target nucleic acid comprises a portion of a nucleic acid from a genomic locus, any DNA amplicon of, a reverse transcribed mRNA, or a cDNA from TSPAN14. SYS1, ARFRP1, or TSPAN14 has been described in more detail in Winter et al., “Genome-wide CRISPR screen reveals novel host factors required for Staphylococcus aureus α-hemolysin-mediated toxicity”, Scientific Reports volume 6, Article number: 24242 (2016), which is hereby incorporated by reference in its entirety.

In some cases, the target nucleic acid comprises a portion of a nucleic acid from a genomic locus, any DNA amplicon of, a reverse transcribed mRNA, or a cDNA from a locus of at least one of: EMC2, EMC3, SEL1L, DERL2, UBE2G2, UBE2J1, and HRD1. In some cases, the target nucleic acid comprises a portion of a nucleic acid from a genomic locus, any DNA amplicon of, a reverse transcribed mRNA, or a cDNA from EMC2. In some cases, the target nucleic acid comprises a portion of a nucleic acid from a genomic locus, any DNA amplicon of, a reverse transcribed mRNA, or a cDNA from EMC3. In some cases, the target nucleic acid comprises a portion of a nucleic acid from a genomic locus, any DNA amplicon of, a reverse transcribed mRNA, or a cDNA from SEL1L. In some cases, the target nucleic acid comprises a portion of a nucleic acid from a genomic locus, any DNA amplicon of, a reverse transcribed mRNA, or a cDNA from DERL2. In some cases, the target nucleic acid comprises a portion of a nucleic acid from a genomic locus, any DNA amplicon of, a reverse transcribed mRNA, or a cDNA from UBE2G2. In some cases, the target nucleic acid comprises a portion of a nucleic acid from a genomic locus, any DNA amplicon of, a reverse transcribed mRNA, or a cDNA from UBE2J1. In some cases, the target nucleic acid comprises a portion of a nucleic acid from a genomic locus, any DNA amplicon of, a reverse transcribed mRNA, or a cDNA from HRD1. EMC2, EMC3, SEL1L, DERL2, UBE2G2, UBE2J1, or HRD1 has been described in more detail in Ma et al., “A CRISPR-Based Screen Identifies Genes Essential for West-Nile-Virus-Induced Cell Death”, Cell Rep. 2015 Jul. 28; 12(4):673-83, which is hereby incorporated by reference in its entirety.

In some instances, samples are used to diagnose a genetic disorder, also referred to as genetic disorder testing. The sample used for genetic disorder testing may comprise at least one target nucleic acid that may bind to an engineered guide nucleic acid of the reagents described herein. In some embodiments, the genetic disorder is hemophilia, sickle cell anemia, β-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHEl, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that may bind to an engineered guide nucleic acid of the reagents described herein. The target nucleic acid, in some cases, is a nucleic acid encoding a sequence associated with a phenotypic trait.

The sample used for genotyping testing may comprise at least one target nucleic acid that may bind to an engineered guide nucleic acid of the reagents described herein. The target nucleic acid, in some cases, is a nucleic acid encoding a sequence associated with a genotype of interest.

The sample may be used for identifying a disease status. For example, a sample is any sample described herein, and is obtained from a subject for use in identifying a disease status of a subject. The disease may be a cancer or genetic disorder. Sometimes, a method comprises obtaining a serum sample from a subject; and identifying a disease status of the subject, and identifying a disease status of the subject. Often, the disease status is prostate disease status, but the status of any disease may be assessed. In any of the embodiments described herein, the device can be configured for asymptomatic, pre-symptomatic, and/or symptomatic diagnostic applications, irrespective of immunity. In any of the embodiments described herein, the device can be configured to perform one or more serological assays on a sample (e.g., a sample comprising blood).

In some embodiments, the sample can be used to identify a mutation in a target nucleic acid of a plant or of a bacteria, virus, or microbe associated with a plant or soil. The devices and methods of the present disclosure can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation.

Any of the above disclosed samples are consistent with the methods, compositions, reagents, enzymes, and systems disclosed herein.

### Numbered Embodiments

Notwithstanding the appended claims, the disclosure sets forth the following numbered embodiments:

1. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to any one of SEQ IDs NO: 1-4.

2. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 1.

3. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 2.

4. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 3.

5. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 4.

6. The composition of any one of embodiments 1-5, wherein the engineered guide nucleic acid comprises a nucleobase sequence that is at least 75%, at least 80%, at least 85%, at least 90%, at least 95%, at least 98%, at least 99%, or 100% identical to GAAUUUCUACUAUUGUAGAU (SEQ ID NO: 55) or UAAUUUCUACUAAGUGUAGAU (SEQ ID NO: 56).

7. The composition of any one of embodiments 1-6, wherein the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21.

8. The composition of any one of embodiments 1-7, wherein the effector protein recognizes a protospacer adjacent motif (PAM) sequence present in a target nucleic acid.

9. The composition of embodiment 8, wherein the PAM sequence is YYN, wherein N is an adenine (A), a guanine (G), a cytosine (C), or a thymine (T); and wherein Y is a C or T.

10. The composition of any one of embodiments 1-9, wherein the composition does not comprise Thermostable Inorganic Pyrophosphatase (TIPP).

11. The composition of any one of embodiments 1-10, wherein the composition further comprises Mg².

12. The composition of any one of embodiments 1-11, wherein the composition has a pH within a range of from about 8.0 to about 9.0.

13. The composition of any one of embodiments 1-12, wherein the composition further comprises a reporter nucleic acid.

14. The composition of any one of embodiments 1-13, wherein the composition has a temperature of at least 45° C.

15. The composition of any one of embodiments 1-14, wherein the effector protein provides higher transcollateral cleavage activity at 70° C. than at 37° C. when a target nucleic acid comprises a single nucleotide polymorphism (SNP).

16. The composition of any one of embodiments 1-15, wherein the effector protein has a threshold of detection of less than 250 μM, less than 25 μM, less than 2.5 μM, or less than 250 fM of the target nucleic acid at a temperature within a range of from about 45° C. to about 80° C.

17. The composition of any one of embodiments 1-16, wherein the target nucleic acid is present in a sample at a concentration of less than 250 μM, less than 25 μM, less than 2.5 μM, or less than 250 fM.

18. The composition of any one of embodiments 1-17, wherein the effector protein has catalytic efficiency of at least about 1.7×10⁷ M⁻¹s⁻¹ at a temperature within a range of about 45° C. to 80° C.

19. A composition comprising an effector protein and an engineered guide nucleic acid, wherein the effector protein has a threshold of detection of less than 250 μM, less than 25 μM, less than 2.5 μM, or less than 250 fM of the target nucleic acid at a temperature within a range of from about 45° C. to about 80° C.

20. A composition comprising an effector protein and an engineered guide nucleic acid, wherein the effector protein has catalytic efficiency of at least about 1.7×10⁷ M⁻¹s⁻¹ at a temperature within a range of from about 45° C. to about 80° C.

21. A composition comprising an effector protein and an engineered guide nucleic acid, wherein the effector protein provides higher transcollateral cleavage activity at 70° C. than at 37° C. when a target nucleic acid comprises a single nucleotide polymorphism (SNP).

22. Use of the composition of any one of embodiments 19-21 in a method of detecting a presence of a target nucleic acid.

23. A composition comprising an effector protein and an engineered guide nucleic acid, wherein the engineered guide nucleic acid comprises: a nucleobase sequence that is at least 75%, at least 80%, at least 85%, at least 90%, at least 95%, at least 98%, at least 99%, or 100% identical to any one of SEQ ID NOs: 19-21.

24. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 1, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 19.

25. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 2, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 20.

26. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 3, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 19.

27. A composition comprising an effector protein and an engineered guide nucleic acid, wherein the effector protein comprises an amino acid sequence that is at least 75%, at least 80%, at least 85%, at least 90%, at least 95%, at least 98%, at least 99%, or 100% identical to SEQ ID NO: 4, and wherein the engineered guide nucleic acid comprises a nucleobase sequence that is at least 75%, at least 80%, at least 85%, at least 90%, at least 95%, at least 98%, at least 99%, or 100% identical to SEQ ID NO: 21.

28. The composition of any one of claims 1-27, wherein the engineered guide nucleic acid further comprises a trans-activating crRNA (tracrRNA).

29. The composition of any one of claims 1-28, wherein the engineered guide nucleic acid is a single guide RNA (sgRNA).

30. A system for detecting a target nucleic acid, comprising the composition of any one of embodiments 1-29 in a solution, wherein the solution comprises at least one of a buffering agent, a salt, a crowding agent, a detergent, a reducing agent, a competitor, and a detection agent.

31. The system of embodiment 30, wherein the pH of the solution is selected from at least about 6.0, at least about 6.5, at least about 7.0, at least about 7.5, at least about 8.0, at least about 8.5, or at least about 9.

32. The system of embodiment 30 or 31, wherein the salt is selected from the group consisting of a magnesium salt, a potassium salt, a sodium salt, and a calcium salt.

33. The system of any one of embodiments 30-32, wherein the concentration of the salt in the solution is selected from at least about 1 millimolar (mM), at least about 3 mM, at least about 5 mM, at least about 7 mM, at least about 9 mM, at least about 11 mM, at least about 13 mM, or at least about 15 mM.

34. The system of any one of embodiments 30-33, wherein the detection reagent is selected from a reporter nucleic acid, a detection moiety, an additional effector protein, an enzyme, or a combination thereof.

35. The system of any one of embodiments 30-34, wherein the detection reagent is a reporter nucleic acid.

36. The system of embodiment 35, wherein the reporter nucleic acid comprises a fluorophore, a quencher, or a combination thereof.

37. The system of claim 35, wherein the reporter nucleic acid comprises the fluorophore.

38. The system of claim 35, wherein the reporter nucleic acid comprises the quencher.

39. The system of any one of embodiments 35-58, wherein the reporter nucleic acid is in the form of single stranded deoxyribonucleic acid (DNA).

40. The system of any one of embodiments 35-39, comprising at least one amplification reagent for amplifying the target nucleic acid.

41. The system of embodiment 40, wherein the at least one amplification reagent is selected from the group consisting of a primer, an activator, a deoxynucleoside triphosphate (dNTP), a ribonucleoside triphosphate (rNTP), and combinations thereof.

42. A method of detecting a target nucleic acid in a sample, comprising: a. contacting the sample with: i. an effector protein, wherein the effector protein comprises an amino acid sequence that is at least 70%, at least 75%, at least 80%, at least 85%, at least 90%, at least 95%, or 100% identical to any one of SEQ ID NOs: 1-4, ii. an engineered guide nucleic acid, and iii. a detection reagent that is cleaved in the presence of the effector protein, the engineered guide nucleic acid, and the target nucleic acid; and b. detecting a signal produced by cleavage of the detection reagent, thereby detecting the target nucleic acid in the sample.

43. The method of embodiment 42, wherein the engineered guide nucleic acid comprises a nucleobase sequence that is at least 75%, at least 80%, at least 85%, at least 90%, at least 95%, at least 98%, at least 99%, or 100% identical to GAAUUUCUACUAUUGUAGAU (SEQ ID NO: 55) or UAAUUUCUACUAAGUGUAGAU (SEQ ID NO: 56).

44. The method of embodiment 42 or 43, wherein the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21.

45. A method of detecting a target nucleic acid in a sample, comprising: a. contacting the sample with: i. an effector protein, wherein the effector protein comprises an amino acid sequence selected from any one of SEQ ID NOS: 1-4, ii. an engineered guide nucleic acid, and iii. a detection reagent that is cleaved in the presence of the effector protein, the engineered guide nucleic acid, and a target nucleic acid, wherein the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T; and b. detecting a signal produced by cleavage of the detection reagent, thereby detecting the target nucleic acid in the sample.

46. The method of any one of embodiments 42-45, wherein the method comprises amplifying the target nucleic acid.

47. The method of embodiment 46, wherein amplifying is performed before contacting.

48. The method of embodiment 46, wherein amplifying is performed during contacting.

49. The method of any one of embodiments 42-48, wherein detecting is performed at a temperature of at least about 40° C., at least about 45° C., at least about 50° C., at least about 55° C., at least about 60° C., or at least about 65° C.

50. The method of any one of embodiments 42-48, wherein detecting is performed at about 40° C., at about 45° C., at about 50° C., at about 55° C., at about 60° C., or at about 65° C.

51. The method of any one of embodiments 46-50, wherein amplifying is performed at a temperature of at least about 40° C., at least about 45° C., at least about 50° C., at least about 55° C., at least about 60° C., or at least about 65° C.

52. The method of any one of embodiments 46-50, wherein amplifying is performed at about 40° C., at about 45° C., at about 50° C., at about 55° C., at about 60° C., or at about 65° C.

53. The method of any one of embodiments 42-52, wherein the engineered guide nucleic acid comprises a crRNA, a tracrRNA, or a combination thereof.

54. The method of any one of embodiments 42-53, wherein the engineered guide nucleic acid is a single guide RNA (sgRNA).

55. The method of any one of embodiments 42-54, wherein the effector protein provides cis-cleavage activity on the target nucleic acid.

56. The method of any one of embodiments 42-55, wherein the effector protein provides transcollateral cleavage activity on the target nucleic acid.

57. The method of embodiment 56, wherein the transcollateral cleavage activity cleaves a single strand of the target nucleic acid.

58. The method of embodiment 57, wherein the transcollateral cleavage activity cleaves the single strand of the target nucleic acid in a sequence non-specific manner.

59. The method of any one of embodiments 42-58, wherein the effector protein, the engineered guide nucleic acid, and reporter nucleic acid are formulated in a solution, wherein the solution comprises at least one of a buffering agent, a salt, a crowding agent, a detergent, a reducing agent, a competitor, and a detection agent.

60. The method of embodiment 59, wherein the pH of the solution is selected from at least about 6.0, at least about 6.5, at least about 7.0, at least about 7.5, at least about 8.0, at least about 8.5, or at least about 9.

61. The method of embodiment 59 or 60, wherein the salt is selected from the group consisting a magnesium salt, a potassium salt, a sodium salt, and a calcium salt.

62. The method of any one of embodiments 59-61, wherein the concentration of the salt in the solution is selected from at least about 1 millimolar (mM), at least about 3 mM, at least about 5 mM, at least about 7 mM, at least about 9 mM, at least about 11 mM, at least about 13 mM, or at least about 15 mM.

63. The method of embodiment 59, wherein the detection reagent is selected from a reporter nucleic acid, a detection moiety, an additional effector protein, or a combination thereof.

64. The method of embodiment 63, wherein the detection reagent is a reporter nucleic acid.

65. The method of embodiment 64, wherein the reporter nucleic acid comprises a fluorophore, a quencher, or a combination thereof.

66. The method of embodiment 64, wherein the reporter nucleic acid comprises the fluorophore.

67. The method of embodiment 64, wherein the reporter nucleic acid comprises the quencher.

68. The method any one of embodiments 65-67, wherein the reporter nucleic acid is in the form of single stranded deoxyribonucleic acid (DNA).

69. The method of any one of embodiments 42-68, further comprising reverse transcribing the target nucleic acid, amplifying the target nucleic acid, in vitro transcribing the target nucleic acid, or any combination thereof.

70. The method of embodiment 69, comprising reverse transcribing the target nucleic acid and/or amplifying the target nucleic acid before contacting the sample with the effector protein, the engineered guide nucleic acid, and the detection reagent.

71. The method of embodiment 69, comprising reverse transcribing the target nucleic acid and/or amplifying the target nucleic acid after contacting the sample with the effector protein, the engineered guide nucleic acid, and the detection reagent.

72. The method of embodiment 69, wherein the contacting and the reverse transcribing are carried out at a same temperature.

73. The method of embodiment 69, wherein the detecting and the reverse transcribing are carried out at a same temperature.

74. The method of embodiment 72 or 73, wherein the contacting, the detecting, and the reverse transcribing are carried out at the same temperature.

75. The method of embodiment 69, wherein the contacting and the amplifying are carried out at a same temperature.

76. The method of embodiment 69, wherein the detecting and the amplifying are carried out at a same temperature.

77. The method of embodiment 75 or 76, wherein the contacting, the detecting, and the amplifying are carried out at the same temperature.

78. The method of embodiment 74 or 77, wherein the contacting, the detecting, the reverse transcribing, and the amplifying are carried out at the same temperature.

79. The method of embodiment 69, wherein the contacting and the reverse transcribing are carried out in a single reaction chamber.

80. The method of embodiment 69, wherein the detecting and the reverse transcribing are carried out in a single reaction chamber.

81. The method of embodiment 79 or 80, wherein the contacting, the detecting, and the reverse transcribing are carried out in a single reaction chamber.

82. The method of embodiment 69, wherein the contacting and the amplifying are carried out in a single reaction chamber.

83. The method of embodiment 69, wherein the detecting and the amplifying are carried out in a single reaction chamber.

84. The method of embodiment 82 or 83, wherein the contacting, the detecting, and the amplifying are carried out at the single reaction chamber.

85. The method of embodiment 81 or 84, wherein the contacting, the detecting, the reverse transcribing, and the amplifying are carried out at the single reaction chamber.

86. The method of any one of embodiments 46-85, wherein amplifying the target nucleic acid comprises at least one amplification reagent.

87. The method of embodiment 86, wherein the at least one amplification reagent is selected from the group consisting of a primer, an activator, a dNTP, an rNTP, and combinations thereof.

88. The method of any one of embodiments 69-87, wherein amplifying the target nucleic acid comprises isothermal amplification.

89. The method of any one of embodiments 42-88, wherein the target nucleic acid is from a pathogen.

90. The method of embodiment 89, wherein the pathogen is a virus or a bacterium.

91. The method of embodiment 90, wherein the pathogen is a virus.

92. The method of embodiment 91, wherein the virus is a SARS-CoV-2 virus or a variant thereof, an influenza A virus, an influenza B virus, a human papillomavirus, a herpes simplex virus, or a combination thereof.

93. The method of embodiment 90, wherein the pathogen is a bacterium.

94. The method of embodiment 93, wherein the bacterium is a Chlamydia trachomatis.

95. The method of any one of embodiments 42-89, wherein the target nucleic acid is from a eukaryote.

96. The method of any one of embodiments 42-95, wherein the target nucleic acid is a ribonucleic acid (RNA).

97. The method of any one of embodiments 42-95, wherein the target nucleic acid is a deoxyribonucleic acid (DNA).

98. The method of any one of embodiments 42-97, wherein the target nucleic acid is from a sample.

99. The method of embodiment 98, wherein the sample comprises blood, serum, plasma, saliva, urine, a mucosal sample, a peritoneal sample, cerebrospinal fluid, gastric secretions, nasal secretions, sputum, pharyngeal exudates, urethral or vaginal secretions, an exudate, an effusion, a tissue sample, or any combination thereof.

100. A method of modifying a target nucleic acid, the method comprising contacting an effector protein and an engineered guide nucleic acid with a target nucleic acid, wherein the effector protein comprises an amino acid sequence that is at least 70%, at least 75%, at least 80%, at least 85%, at least 90%, at least 95%, or 100% identical to any one of SEQ ID NOs: 1-4, thereby modifying the target nucleic acid.

101. The method of embodiment 100, wherein the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21.

102. The method of embodiment 100 or 101, wherein the target nucleic acid comprises a protospacer adjacent motif (PAM) sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T, thereby modifying the target nucleic acid.

103. The method of any one of embodiments 100-102, wherein modifying the target nucleic acid comprises cleaving the target nucleic acid, deleting a nucleotide of the target nucleic acid, inserting a nucleotide into the target nucleic acid, substituting a nucleotide of the target nucleic acid with a donor nucleotide or an additional nucleotide, or any combination thereof.

104. The method of any one of embodiments 100-103, wherein modifying the target nucleic acid comprises substituting the nucleotide of the target nucleic acid with the donor nucleotide or the additional nucleotide.

105. The method of any one of embodiments 100-104, wherein the method comprises contacting the target nucleic acid with the donor nucleic acid.

106. The method of any one of embodiments 100-105, wherein the target nucleic acid comprises a mutation associated with a disease.

107. The method of embodiment 106, wherein the mutation is suspected to cause the disease.

108. The method of embodiment 106 or 107, wherein the disease comprises, at least in part, a cancer, an inherited disorder, an ophthalmological disorder, a neurological disorder, a blood disorder, a metabolic disorder, or a combination thereof.

109. The method of embodiment 108, wherein the disease comprises, at least in part, a neurological disorder.

110. The method of embodiment 108 or 109, wherein the neurological disorder comprises Duchenne muscular dystrophy, myotonic dystrophy Type 1, or cystic fibrosis.

111. The method of embodiment 108, wherein the neurological disorder is a neurodegenerative disease.

112. The method of any one of embodiments 100-111, wherein the target nucleic acid is encoded by a gene selected from DNMT1, HPRT1, RPL32P3, CCR5, FANCF, GRIN2B, EMX1, AAVS1, ALKBH5, CLTA, CDK11, CTNNB1, AXIN1, LRP6, TBK1, BAP1, TLE3, PPM1A, BCL2L2, SUFU, RICTOR, VPS35, TOP1, SIRT1, PTEN, MMD, PAQR8, H2AX, POU5F1, OCT4, SYS1, ARFRP1, TSPAN14, EMC2, EMC3, SEL1L, DERL2, UBE2G2, UBE2J1, and HRD1.

113. The method of any one of embodiments 100-102, wherein the target nucleic acid is PCSK9, or a portion thereof.

114. The method of any one of embodiments 100-102, wherein the target nucleic acid is TRAC, B2M, PD1, or a portion thereof.

115. The method of any one of embodiments 100-102, wherein contacting the effector protein and the engineered guide nucleic acid with the target nucleic acid occurs in vitro, in vivo, or ex vivo.

116. A method of generating a recombinant cell, the method comprising providing an effector protein and an engineered guide nucleic acid to a target cell, wherein the effector protein comprises an amino acid sequence that is at least 70%, at least 75%, at least 80%, at least 85%, at least 90%, at least 95%, or 100% identical to any one of SEQ ID NOs: 1-4, thereby generating the recombinant cell from the target cell.

117. The method of embodiment 116, wherein the engineered guide nucleic acid comprises a nucleobase sequence that is at least 75%, at least 80%, at least 85%, at least 90%, at least 95%, at least 98%, at least 99%, or 100% identical to any one of SEQ ID NOs: 19-21.

118. The method of embodiment 116 or 117, wherein the target nucleic acid comprises a protospacer adjacent motif (PAM) sequence directly adjacent to a target sequence of a target nucleic acid, wherein the PAM sequence is selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T, thereby generating the recombinant cell from the target cell.

119. The method of any one of embodiments 116-118, comprising providing a nucleic acid encoding the effector protein.

120. The method of embodiment 119, wherein the nucleic acid encodes the engineered guide nucleic acid.

121. The method of any one of embodiments 116-120, wherein providing the effector protein and the engineered guide nucleic acid to the target cell comprises electroporation, acoustic poration, optoporation, viral vector-based delivery, induced transduction by osmocytosis and propanebetaine (iTOP), nanoparticle delivery, cell-penetrating peptide (CPP) delivery, DNA nanostructure delivery, or any combination thereof.

122. The method of embodiment 121, wherein the nanoparticle delivery comprises lipid nanoparticle delivery or gold nanoparticle delivery.

123. The method of any one of embodiments 116-122, wherein providing the effector protein and the engineered guide nucleic acid to the target cell generates a double-stranded break in the genome of the target cell, and optionally, wherein the method comprises detecting the double-stranded break.

124. The method of embodiment 123, comprising repairing the double-stranded break, and wherein the repairing results in an insertion-deletion (indel) in the genome of the target cell.

125. The method of embodiment 124, further comprising delivering a donor nucleic acid to the target cell.

126. The method of embodiment 125, wherein the donor nucleic acid is incorporated into the genome of the target cell, and optionally wherein the method comprises detecting the incorporation of the donor nucleic acid in the genome of the target cell.

127. The method of any one of embodiments 116-126, wherein the target cell is a eukaryotic cell.

128. The method of embodiment 127, wherein the target cell is a mammalian cell.

129. The method of embodiment 127, wherein the target cell is a cancer cell, an animal cell, an HEK293 cell, or an immune cell.

130. The method of embodiment 127, wherein the target cell is a Chinese hamster ovary cell.

131. The method of any one of embodiments 116-126, wherein the target cell is a prokaryotic cell.

132. A recombinant cell generated by the method of any one of embodiments 116-131.

133. The recombinant cell of embodiment 132, wherein the recombinant cell is a T cell.

134. The recombinant cell of embodiment 133, wherein the T cell is a natural killer T (NKT) cell.

135. The recombinant cell of embodiment 134, wherein the recombinant cell is an induced pluripotent stem cell (iPS).

136. A population of recombinant cells generated by the method of any one of embodiments 116-131.

137. The population of recombinant cells of embodiment 136, wherein the population of recombinant cells comprises a T cell.

138. The population of recombinant cells of embodiment 137, wherein the T cell is an NKT cell.

139. The population of recombinant cells of embodiment 138, wherein the population of recombinant cells comprises an iPS.

140. A progeny cell of the recombinant cell of any one of embodiments 132-135.

141. The progeny cell of embodiment 140, wherein the progeny cell is a T cell.

142. The progeny cell of embodiment 141, wherein the T cell is a natural killer T (NKT) cell.

143. The progeny cell of embodiment 142, wherein the progeny cell is an induced pluripotent stem cell (iPS).

144. A composition comprising an effector protein and an engineered guide nucleic acid, wherein the effector protein provides transcollateral cleavage activity upon binding of the engineered guide nucleic acid to a target nucleic acid at a temperature within a range of from about 45° C. to about 80° C.

145. The composition of embodiment 144, wherein the effector protein comprises an amino acid sequence that is at least 75%, at least 80%, at least 85%, at least 90%, at least 95%, at least 98%, at least 99%, or 100% identical to SEQ ID NO: 2.

146. The composition of embodiment 144 or 145, further comprising Mg²+.

147. The composition of any one of embodiments 144-146, wherein the composition has a pH within a range of from about 8.0 to about 9.0.

148. The composition of any one of embodiments 144-147, wherein the composition further comprises a reporter nucleic acid.

149. The composition of any one of embodiments 144-148, wherein the composition has a temperature of at least 45° C.

150. The composition of any one of embodiments 144-149, wherein the effector protein provides higher transcollateral cleavage activity at 70° C. than at 37° C. when the target nucleic acid comprises a single nucleotide polymorphism (SNP).

151. The composition of any one of embodiments 144-150, wherein the effector protein has a threshold of detection of less than 250 μM, less than 25 μM, less than 2.5 μM, or less than 250 fM of the target nucleic acid at a temperature within a range of about 45° C. to 80° C.

152. The composition of any one of embodiments 144-151, wherein the target nucleic acid is present in a sample at a concentration of less than 250 μM, less than 25 μM, less than 2.5 μM, or less than 250 fM.

153. The composition of any one of embodiments 144-152, wherein the effector protein has catalytic efficiency of at least about 1.7×10⁷ M⁻¹s⁻¹ at a temperature within a range of from about 45° C. to about 80° C.

154. A method of detecting a target nucleic acid, the method comprising: a. contacting the sample with: i. an effector protein, wherein the effector protein provides transcollateral cleavage activity on a target nucleic acid at a temperature within a range of about 45° C. to 80° C., ii. an engineered guide nucleic acid, and iii. a detection reagent that is cleaved in the presence of the effector protein, the engineered guide nucleic acid, and the target nucleic acid; and b. detecting a signal indicative of cleavage of the detection reagent, thereby detecting the target nucleic acid.

155. The method of embodiment 154, wherein the effector protein comprises an amino acid sequence that is at least 75%, at least 80%, at least 85%, at least 90%, at least 95%, at least 98%, at least 99%, or 100% identical to SEQ ID NO: 2.

156. The method of embodiment 154 or 155, wherein the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T.

157. Use of a composition comprising: a. heating a sample to a temperature within a range of about 45° C. to 80° C.; and b. contacting the heated sample with a composition comprising: i. an effector protein, wherein the effector protein provides transcollateral cleavage activity on a target nucleic acid at a temperature within a range of about 45° C. to 80° C., ii. an engineered guide nucleic acid, and iii. a detection reagent that is cleaved in the presence of the effector protein, the engineered guide nucleic acid, and a target nucleic acid, wherein the contacting happens at a temperature above 45° C.

158. The use of embodiment 157, wherein the effector protein comprises an amino acid sequence that is at least 75%, at least 80%, at least 85%, at least 90%, at least 95%, at least 98%, at least 99%, or 100% identical to SEQ ID NO: 2.

159. The use of embodiment 157 or 158, wherein the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T.

160. The use of an effector protein to detect a target nucleic acid in a sample according to the method of any one of embodiments 45-99 and 154-156.

161. The use of an effector protein to modify a target nucleic acid according to the method of any one of embodiments 100-115.

162. The use of an effector protein to generate a recombinant cell according to the method of any one of embodiments 116-131.

163. A vector encoding an effector protein comprising an amino acid sequence that is at least 75%, at least 80%, at least 85%, at least 90%, at least 95%, at least 98%, at least 99%, or 100% identical to any one of SEQ ID NOs: 1-4.

164. An engineered guide nucleic acid comprising a crRNA comprising a nucleobase sequence that is at least 75%, at least 80%, at least 85%, at least 90%, at least 95%, at least 98%, at least 99%, or 100% identical to any one of SEQ ID NOs: 19-21.

165. A vector encoding the engineered guide nucleic acid of embodiment 164.

166. A vector encoding an effector protein and an engineered guide nucleic acid, wherein a. the effector protein comprises an amino acid sequence that is at least 75%, at least 80%, at least 85%, at least 90%, at least 95%, at least 98%, at least 99%, or 100% identical to any one of SEQ ID NOs: 1-4; and b. the engineered guide nucleic acid comprises a crRNA comprising a nucleobase sequence that is at least 75%, at least 80%, at least 85%, at least 90%, at least 95%, at least 98%, at least 99%, or 100% identical to any one of SEQ ID NOs: 19-21.

167. The vector of embodiment 166, wherein the effector protein comprises an amino acid sequence that is at least 75%, at least 80%, at least 85%, at least 90%, at least 95%, at least 98%, at least 99%, or 100% identical to SEQ ID NO: 2, and the engineered guide nucleic acid comprises a crRNA comprising a nucleobase sequence that is at least 75%, at least 80%, at least 85%, at least 90%, at least 95%, at least 98%, at least 99%, or 100% identical to SEQ ID NO: 20.

168. The vector of embodiment 166 or 167, wherein the vector further comprises or encodes a donor nucleic acid.

169. A vector system comprising the vector of any one of claims and any one of embodiments 165-167 and the vector of embodiment 168.

170. The vector system of embodiment 169, wherein the vector system further comprises a vector comprising or encoding a donor nucleic acid.

171. A cell comprising the vector, engineered guide nucleic acid, or vector system of any one of embodiments 163-169.

172. A complex comprising an effector protein comprising an amino acid sequence that is at least 75%, at least 80%, at least 85%, at least 90%, at least 95%, at least 99% or 100% identical to any of SEQ ID NOs: 1-4, and an engineered guide nucleic acid comprising a crRNA comprising a nucleobase sequence that is at least 75%, at least 80%, at least 85%, at least 90%, at least 95%, at least 97%, or 100% identical to any of SEQ ID NOs: 19-21.

173. The complex of embodiment 172, wherein the effector protein comprises an amino acid sequence that is at least 75%, at least 80%, at least 85%, at least 90%, at least 95%, at least 99% or 100% identical to SEQ ID NO: 2, and the engineered guide nucleic acid comprises a crRNA comprising a nucleobase sequence that is at least 75%, at least 80%, at least 85%, at least 90%, at least 95%, at least 97%, or 100% identical to SEQ ID NOs: 20.

174. A method of detecting a target nucleic acid in a sample, comprising a. contacting the sample with i. the complex of embodiment 172 or 173, wherein the complex binds to the target sequence; and ii. a detection reagent that is cleaved by the effector protein upon binding to the target sequence; b. detecting a signal produced by cleavage of the detection reagent, thereby detecting the target nucleic acid in the sample.

175. The method of embodiment 174, wherein the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T.

176. The method of embodiment 174 or 175, wherein the method comprises amplifying the target nucleic acid.

177. The method of embodiment 176, wherein the amplifying is performed before and/or during contacting.

178. The method of any one of embodiments 174-177, wherein the detecting is performed at a temperature of at least 40° C., at least 45° C., at least 50° C., at least 55° C., at least 60° C. or at least 65° C.

179. The method of any one of embodiments 174-177, wherein the detecting is performed at a temperature of about 40° C., about 45° C., about 50° C., about 55° C., about 60° C., or about 65° C.

180. The method of any one of embodiments 176-179, wherein the amplifying is performed at a temperature of at least 40° C., at least 45° C., at least 50° C., at least 55° C., at least 60° C. or at least 65° C.

181. The method of any one of embodiments 176-179, wherein the amplifying is performed at a temperature of about 40° C., about 45° C., about 50° C., about 55° C., about 60° C., or about 65° C.

182. The method of any one of embodiments 174-181, wherein the complex and the detection reagent are formulated in a solution, wherein the solution comprises at least one of a buffering agent, a salt, a crowding agent, a detergent, a reducing agent, a competitor, and a detection agent.

183. The method of embodiment 202, wherein the pH of the solution is selected from at least about 6.0, at least about 6.5, at least about 7.0, at least about 7.5, at least about 8.0, at least about 8.5, or at least about 9.

184. The method of embodiment 183, wherein the pH of the solution is at least 7.0.

185. The method of embodiment 184, wherein the pH of the solution is at least 7.5.

186. The method of any one of embodiments 182 to 185, wherein the salt is selected from the group consisting a magnesium salt, a potassium salt, a sodium salt, and a calcium salt.

187. The method of any one of embodiments 182 to 186, wherein the concentration of the salt in the solution is selected from at least about 1 millimolar (mM), at least about 3 mM, at least about 5 mM, at least about 7 mM, at least about 9 mM, at least about 11 mM, at least about 13 mM, or at least about 15 mM.

188. The method of any one of embodiments 182 to 187, wherein the detection reagent is the reporter nucleic acid.

189. The method of embodiment 188, wherein the reporter nucleic acid comprises a fluorophore, a quencher, or a combination thereof.

190. The method of embodiment 189, wherein the reporter nucleic acid comprises a fluorophore.

191. The method of embodiment 189, wherein the reporter nucleic acid comprises a quencher.

192. The method of any one of embodiments 189-191, wherein the reporter nucleic acid is in the form of single stranded deoxyribonucleic acid (DNA).

193. The method of any one of embodiments 174-192, wherein the engineered guide nucleic acid comprises a crRNA, a tracrRNA, or a combination thereof.

194. The method of any one of embodiments 174-193, wherein the engineered guide nucleic acid is a single guide RNA (sgRNA).

195. The method of any one of embodiments 174-194, wherein the effector protein provides cis-cleavage activity on the target nucleic acid.

196. The method of any one of embodiments 174-195, wherein the effector protein provides transcollateral cleavage activity on the target nucleic acid.

197. The method of embodiment 196, wherein the transcollateral cleavage activity cleaves a single strand of the target nucleic acid.

198. The method of embodiment 197, wherein the transcollateral cleavage activity cleaves the single strand of the target nucleic acid in a sequence non-specific manner.

199. The method of any one of embodiments 174-198, further comprising reverse transcribing the target nucleic acid, amplifying the target nucleic acid, in vitro transcribing the target nucleic acid, or any combination thereof.

200. The method of embodiment 199, comprising reverse transcribing the target nucleic acid and/or amplifying the target nucleic acid before contacting the sample with the effector protein, the engineered guide nucleic acid, and the detection reagent.

201. The method of embodiment 199, comprising reverse transcribing the target nucleic acid and/or amplifying the target nucleic acid after contacting the sample with the effector protein, the engineered guide nucleic acid, and the detection reagent.

202. The method of embodiment 199, wherein the contacting and the reverse transcribing are carried out at a same temperature.

203. The method of embodiment 199, wherein the detecting and the reverse transcribing are carried out at a same temperature.

204. The method of embodiment 202 or 203, wherein the contacting, the detecting, and the reverse transcribing are carried out at the same temperature.

205. The method of embodiment 199, wherein the contacting and the amplifying are carried out at a same temperature.

206. The method of embodiment 199, wherein the detecting and the amplifying are carried out at a same temperature.

207. The method of embodiment 205 or 206, wherein the contacting, the detecting, and the amplifying are carried out at the same temperature.

208. The method of embodiment 204 or 207, wherein the contacting, the detecting, the reverse transcribing, and the amplifying are carried out at the same temperature.

209. The method of embodiment 199, wherein the contacting and the reverse transcribing are carried out in a single reaction chamber.

210. The method of embodiment 199, wherein the detecting and the reverse transcribing are carried out in a single reaction chamber.

211. The method of embodiment 209 or 210, wherein the contacting, the detecting, and the reverse transcribing are carried out in a single reaction chamber.

212. The method of embodiment 199, wherein the contacting and the amplifying are carried out in a single reaction chamber.

213. The method of embodiment 199, wherein the detecting and the amplifying are carried out in a single reaction chamber.

214. The method of embodiment 212 or 213, wherein the contacting, the detecting, and the amplifying are carried out at the single reaction chamber.

215. The method of embodiment 211 or 214, wherein the contacting, the detecting, the reverse transcribing, and the amplifying are carried out at the single reaction chamber.

216. The method of any one of embodiments 176-215, wherein amplifying the target nucleic acid comprises at least one amplification reagent.

217. The method of embodiment 216, wherein the at least one amplification reagent is selected from the group consisting of a primer, an activator, a dNTP, an rNTP, and combinations thereof.

218. The method of any one of embodiments 176-217, wherein amplifying the target nucleic acid comprises isothermal amplification.

219. The method of any one of embodiments 174-218, wherein the target nucleic acid is from a pathogen.

220. The method of embodiment 219, wherein the pathogen is a virus or a bacterium.

221. The method of embodiment 220, wherein the pathogen is a virus

222. The method of embodiment 221, wherein the virus is a SARS-CoV-2 virus or a variant thereof, an influenza A virus, an influenza B virus, a human papillomavirus, a herpes simplex virus, or a combination thereof.

223. The method of embodiment 219, wherein the pathogen is a bacterium.

224. The method of embodiment 223, wherein the bacterium is a Chlamydia trachomatis.

225. The method of any one of embodiments 174-224, wherein the target nucleic acid is a ribonucleic acid (RNA).

226. The method of any one of embodiments 174-224, wherein the target nucleic acid is a deoxyribonucleic acid (DNA).

227. The method of any one of embodiments 174-226, wherein the target nucleic acid is from a sample.

228. The method of embodiment 227, wherein the sample comprises blood, serum, plasma, saliva, urine, a mucosal sample, a peritoneal sample, cerebrospinal fluid, gastric secretions, nasal secretions, sputum, pharyngeal exudates, urethral or vaginal secretions, an exudate, an effusion, a tissue sample, or any combination thereof.

229. The use of a complex to detect a target nucleic acid in a sample according to the method of any one of embodiments 174-228.

### EXAMPLES

The following examples are included for illustrative purposes only and are not intended to limit the scope of the disclosure. It will be understood by those of skill in the art that numerous and various modifications can be made to yield essentially similar results without departing from the spirit of the present disclosure.

### Example 1: In Vitro Enrichment Screening Assay

Effector protein proteins and guide RNA combinations represented in TABLE 5 were screened by in vitro enrichment (IVE) for PAM recognition. TABLE 5 shows the components of each effector-guide RNA complex assayed for PAM recognition. Briefly, 10×effector proteins were complexed with corresponding 500 nM guide RNAs for 10 minutes at 37° C. in 1× Cutsmart buffer (50 mM Potassium acetate, 20 mM Tris-acetate, 10 mM Magnesium acetate, 100 μg/ml BSA, pH 7.9 at 25° C.). A 1/10 dilution made with 1× Cutsmart buffer. 10 μL of the complexes were added to 100 μL cutting reactions to give a final concentration of 50 nM. 1,000 ng of a PAM library target plasmid was incubated with the RNP for 15 minutes at 25° C., 45 minutes at 37° C., and 15 minutes at 45° C. Reactions were terminated with 1 μl of proteinase K and 5 μl of 500 mM EDTA for 30 minutes at 37° C. Next generation sequencing (NGS) was performed on cut sequences to identify enriched PAMs. As shown in TABLE 5, cis cleavages were observed with RNP complexes comprising effector protein proteins and corresponding guide RNAs.

TABLE 5
Observed Trans Cleavage for Effector/
crRNA Combination

| Effector | Cis | | |
|---|---|---|---|
| protein | cleavage | | Enriched PAM |
| (SEQ | activity | | identified by |
| ID NO) | (yes/no) | crRNA | NGS * |
| CasM.21544 | yes | SEQ ID | NNNNNYN |
| (SEQ ID | | NO: 19 | (SEQ ID |
| NO: 1) | | | NO: 42) |
| CasM.21526 | yes | SEQ ID | NNNNYYN |
| (SEQ ID | | NO: 20 | (SEQ ID |
| NO: 2) | | | NO: 43) |
| CasM.21550 | yes | SEQ ID | NNNNYTN |
| (SEQ ID | | NO: 19 | (SEQ ID |
| NO: 3) | | | NO: 44) |
| CasM.21530 | yes | SEQ ID | NNNNYNN |
| (SEQ ID | | NO: 21 | (SEQ ID |
| NO: 4) | | | NO: 45) |

* N is A, C, G, or T; Y is C or T

### Example 2: Trans Cleavage Activity of Effector Proteins at 37° C.

Effector proteins were tested for trans cleavage at 37° C. Briefly, partially purified (nickel-NTA purified) effector protein proteins complexed with crRNA were incubated with 2 nM of a TTTG PAM, spacer target on a 1.1 kb fragment in buffer containing 20 mM Tricine (pH=9), 15 mM Mg(OAc)₂, 0.2 mg/ml BSA, 1 mM TCEP at 37° C. 0.01%, 0.1% and 1% dilutions of a Ni-NTA preparation from 50 mL culture were assayed. The crRNA was present at 50 nM. The reporter was 12T (5′ FAM-TTTTTTTTTTTT (SEQ ID NO: 34) −3IABkFQ) present at 200 nM.

Trans cleavage activity was detected by fluorescence signal upon cleavage of a fluorophore-quencher reporter in a DETECTR reaction. The ratio of maximum rates derived from time course experiments of the reaction with target over the no target control reaction (fold on/off for those with trans-cleavage) for the dilution where this ratio was highest is summarized in TABLE 6 below. These ratios are not necessarily proportional to the corresponding effector activity because it is highly dependent on the non-specific nuclease activity present in the crude preparation. However, it expresses the level of confidence a given enzyme has activity under these conditions. Further, the background nuclease activity is eliminated at higher temperatures resulting in the observed increase in on/off ratios. CasM.21530 was used as a negative control for the effector protein. The sequence of Cas12 Variant is SEQ ID NO: 40:

Cas12 Variant-
SEQ ID NO: 40
MKKIDNFVGCYPVSKILRFKAIPIGKTQENIEKKRLVEEDEVRAK
DYKAVKKLIDRYHREFIEGVLDNVKLDGLEEYYMLENKSDREESD
NKKIEIMEERFRRVISKSFKNNEEYKKIFSKKIIEEILPNYIKDE
EEKELVKGFKGFYTAFVGYAQNRENMYSDEKKSTAISYRIVNENM
PRFITNIKVFEKAKSILDVDKINEINEYILNNDYYVDDFFNIDFF
NYVLNQKGIDIYNAIIGGIVTGDGRKIQGLNECINLYNQENKKIR
LPQFKPLYKQILSESESMSFYIDEIESDDMLIDMLKESLQIDSTI
NNAIDDLKVLENNIFDYDLSGIFINNGLPITTISNDVYGQWSTIS
DGWNERYDVLSNAKDKESEKYFEKRRKEYKKVKSFSISDLQELGG
KDLSICKKINEIISEMIDDYKSKIEEIQYLFDIKELEKPLVTDLN
KIELIKNSLDGLKRIERYVIPFLGTGKEQNRDEVFYGYFIKCIDA
IKEIDGVYNKTRNYLTKKPYSKDKFKLYFENPQLMGGWDRNKESD
YRSILLRKNGKYYVAIIDKSSSNCMMNIEEDENDNYEKINYKLLP
GPNKMLPKVFFSKKNREYFAPSKEIERIYSTGTFKKDTNFVKKDC
ENLITFYKDSLDRHEDWSKSEDESFKESSAYRDISEFYRDVEKQG
YRVSFDLLSSNAVNTLVEEGKLYLFQLYNKDFSEKSHGIPNLHTM
YFRSLEDDNNKGNIRLNGGAEMFMRRASLNKQDVTVHKANQPIKN
KNLLNPKKTITLPYDVYKDKRFTEDQYEVHIPITMNKVPNNPYKI
NHMVREQLVKDDNPYVIGIDRGERNLIYVVVVDGQGHIVEQLSLN
EIINENNGISIRTDYHILLDAKERERDESRKQWKQIENIKELKEG
YISQVVHKICELVEKYDAVIALEDLNSGFKNSRVKVEKQVYQKFE
KMLITKLNYMVDKKKDYNKPGGVLNGYQLTTQFESFSKMGTQNGI
MFYIPAWLISKMDPTTGFVDLLKPKYKNKADAQKFFSQFDSIRYD
NQEDAFVEKVNYTKFPRIDADYNKEWEIYTNGERIRVFRNPKKNN
EYDYETVNVSERMKELEDSYDLLYDKGELKETICEMEESKFFEEL
IKLERLTLQMRNSISGRIDVDYLISPVKNSNGYFYNSNDYKKEGA
KYPKDADANGAYNIARKVLWAIEQFKMADEDKLDKTKISIKNQEW
LEYAQTHCE

TABLE 6
Observed Trans Cleavage for Effector Proteins at 37° C.

| Effector protein | Fold on/off for those with trans |
|---|---|
| (SEQ ID NO) | cleavage over no target at 37° C. |
| CasM.21544 (SEQ ID NO: 1) | 39.35 |
| CasM.21526 (SEQ ID NO: 2) | 60.68 |
| CasM.21550 (SEQ IDNO: 3) | 13.63 |
| CasM.21530 (SEQ ID NO: 4) | 3.29 |

### Example 3: Trans Cleavage Activity of Effector Proteins at High Temperatures

Effector proteins were tested for trans cleavage at various temperatures. Briefly, partially purified (nickel-NTA purified) effector protein proteins complexed with crRNA were incubated 2 nM of a TTTG PAM, spacer target on a 1.1 kb fragment in buffer containing 20 mM Tricine (pH=9), 15 mM Mg(OAc)₂, 0.2 mg/ml BSA, 1 mM TCEP at 40° C., 45° C., 50° C., 55° C., 60° C., or 65° C. 0.01%, 0.1% and 1% dilutions of a Ni-NTA preparation from 50 mL culture were assayed. The crRNA was present at 50 nM. The reporter was 12T (5′ FAM-TTTTTTTTTTTT (SEQ ID NO: 34) −3IABkFQ) present at 200 nM.

Trans cleavage activity was detected by fluorescence signal upon cleavage of a fluorophore-quencher reporter in a DETECTR reaction. The ratio of maximum rates derived from time course experiments of the reaction with target over the no target control reaction (fold on/off for those with trans-cleavage) and the maximum cleavage rate is summarized in TABLE 7 below. CasM.21530 was used as a negative control for the effector protein.

TABLE 7
Observed Trans Cleavage for Effector Proteins

| | 40° C. | 45° C. | 50° C. | 55° C. | 60° C. | 65° C. |
|---|---|---|---|---|---|---|
| CasM.21544 (SEQ ID NO: 1) | 2.04 | 1.98 | 3.9 | 2.38 | 2.39 | 0.48 |
| CasM.21526 (SEQ ID NO: 2) | 8.71 | 10.52 | 6.86 | 12.02 | 105.09 | 111.32 |
| CasM.21550 (SEQ IDNO: 3) | 1.53 | 1.55 | 2.78 | 3.71 | 22.12 | 57.19 |
| CasM.21530 (SEQ ID NO: 4) | 0.82 | 1.16 | 0.64 | 1.13 | 4.37 | 3.92 |

### Example 4: Thermostability of Effector Proteins

Effector proteins were tested for thermostability. RNPs were formed in standard diluent (20 mM HEPES, pH 7.5, 0.2 mg/mL BSA, 1 mM TCEP) by mixing 4×protein and crRNA for 20 mins at room temperature. The 1×concentration of proteins was 40 nM and the final concentration of crRNAs was 50 nM. 5 uL of these 4×RNPs were plated and a 15 uL 1.33× mix of the following components was then added for a total volume of 20 uL (listed at final concentration): 1× Cutsmart buffer, or 1× trans cleavage buffer, 0.1 nM TTTG S1 target (1.1 kb fragment), “12T” reporter (5′ FAM-TTTTTTTTTTTT (SEQ ID NO: 34) −3IABkFQ) at 200 nM. Reactions were carried out (n=4 per condition) at 40° C., 45° C., 50° C., 55° C., 60° C., 65° C., 70° C., 75° C., 80° C., 85° C., or 90° C. Time course data was collected for 30 minutes.

Trans cleavage activity was detected by fluorescence signal upon cleavage of a fluorophore-quencher reporter in a DETECTR reaction. The ratio of maximum rates derived from time course experiments of the reaction with target over the no target control reaction (fold on/off for those with trans-cleavage) and the maximum cleavage rate is summarized in TABLE 8 below. CasM.21530 was used as a negative control for the effector protein. The maximum trans cleavage rate was measured by the arbitrary unit of the fluorescence of the DETECTR reaction in two different buffer solutions: (Buffer 1) 20 mM tricine (pH 9.0), 15 mM Mg(OAc)₂; and (Buffer 2) 50 mM Potassium acetate, 20 mM Tris-acetate, 10 mM Magnesium acetate, 100 μg/ml BSA, pH 7.9 at 25° C. (also referred to as “NEB cutsmart buffer”). Cas12 Variant (SEQ ID NO: 40) was used as a negative control for the effector protein. Results are summarized in TABLE 8 below.

TABLE 8
Observed Trans Cleavage for Effector Proteins

| Effector | | | | | | | | | | | |
|---|---|---|---|---|---|---|---|---|---|---|---|
| protein | | | | | | | | | | | |
| (SEQ ID | Maximum trans cleavage rate (arbitrary unit) measured at (° C.) | | | | | | | | | | |
| NO) | 40 | 45 | 50 | 55 | 60 | 65 | 70 | 75 | 80 | 85 | 90 |

Buffer 1 (20 mM tricine (pH 9.0), 15 mM Mg(OAc)₂)

| | | | | | | | | | | | |
|---|---|---|---|---|---|---|---|---|---|---|---|
| CasM.21526 | 609,838 | 778,906 | 849,662 | 922,738 | 950,538 | 1,195,578 | 865,408 | 478,664 | 75,574 | 8,450 | 7,711 |
| (SEQ ID NO: 2) | | | | | | | | | | | |
| Cas12 | 31,823 | 12,259 | 5,115 | 6,137 | 5,403 | 8,347 | 5,825 | 6,126 | 8,534 | 8,536 | 6,656 |
| Variant | | | | | | | | | | | |
| (SEQ ID | | | | | | | | | | | |
| NO: 40) | | | | | | | | | | | |

Buffer 2 (50 mM Potassium acetate, 20 mM Tris-acetate, 10 mM Magnesium acetate,
100 μg/ml BSA, pH 7.9 at 25° C.)

| | | | | | | | | | | | |
|---|---|---|---|---|---|---|---|---|---|---|---|
| CasM.21526 | 47,058 | 57,937 | 51,642 | 47,406 | 36,435 | 21,867 | 12,769 | 5,811 | 6,861 | 4,987 | 2,935 |
| (SEQ ID NO: 2) | | | | | | | | | | | |
| Cas12 | 96,023 | 39,639 | 8,116 | 6,311 | 7,493 | 6,894 | 4,564 | 3,420 | 6,266 | 5,269 | 6,905 |
| Variant | | | | | | | | | | | |
| (SEQ ID | | | | | | | | | | | |
| NO: 40) | | | | | | | | | | | |

### Example 5: Buffer Optimization of Effector Proteins

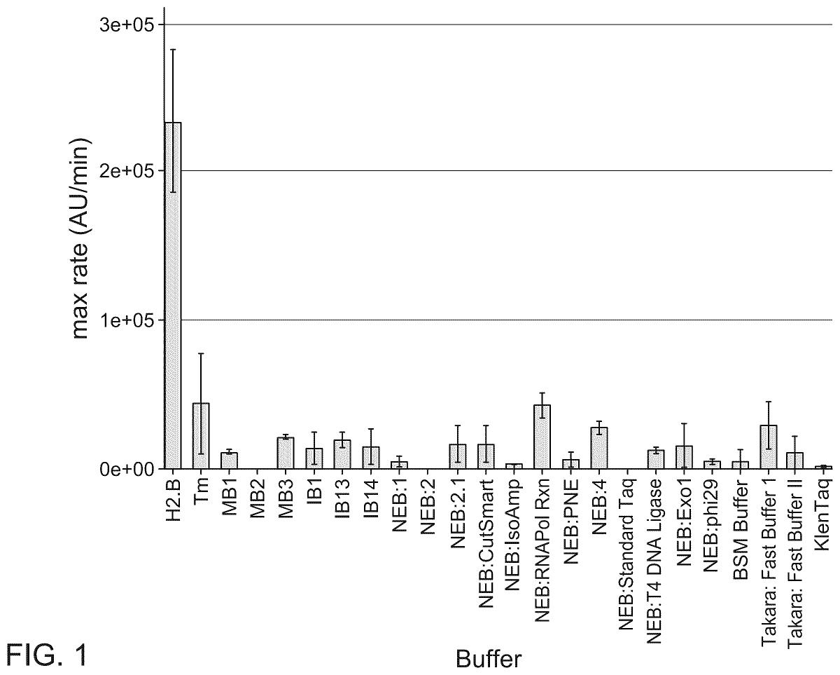

Effector proteins were tested for trans cleavage in various buffer conditions. Briefly, CasM.21526 (SEQ ID NO: 2) effector proteins were complexed with crRNA for 15 minutes at 37° C. The 1×concentration of proteins was 40 nM and the final concentration of crRNAs was 40 nM. 2 uL of these RNPs was combined with a 6 uL mix of the following components for a total volume of 8 uL (listed at final concentration): trans cleavage buffer, target dsDNA (500 μM), and FQ reporter (200 nM). Reactions were carried out at 35° C. for 60 minutes. Trans cleavage activity was detected by fluorescence signal upon cleavage of a fluorophore-quencher reporter in a DETECTR reaction. The maximum rates derived from time course experiments of the reaction with target are summarized in FIG. 1. FIG. 1 shows enhanced trans cleavage performance of CasM.21526 (SEQ ID NO: 2) in various buffers. CasM.21526 (SEQ ID NO: 2) appeared to favor basic buffers with high Mg2+, minimal salt, and the presence of BSA under the conditions tested.

The crRNAs used in Examples 5-15 were:

(SEQ ID NO: 52)
GAAUUUCUACUAUUGUAGAUGCCGAUAAUGAUGUAGGGAU
(Mammuthus, PAM: TTTG)
(SEQ ID NO: 53)
UAAUUUCUACUAAGUGUAGAUgccgauaaugauguagggau
(Mammuthus, PAM: TTTG, CasM08 repeat)
(SEQ ID NO: 54)
UAAUUUCUACUAAGUGUAGAUCCCCCAGCGCUUCAGCGUUC
(SARS-CoV-2 N-gene,
PAM: TTTG, CasM08 repeat).

### Example 6: Performance of Effector Proteins with Various Additives

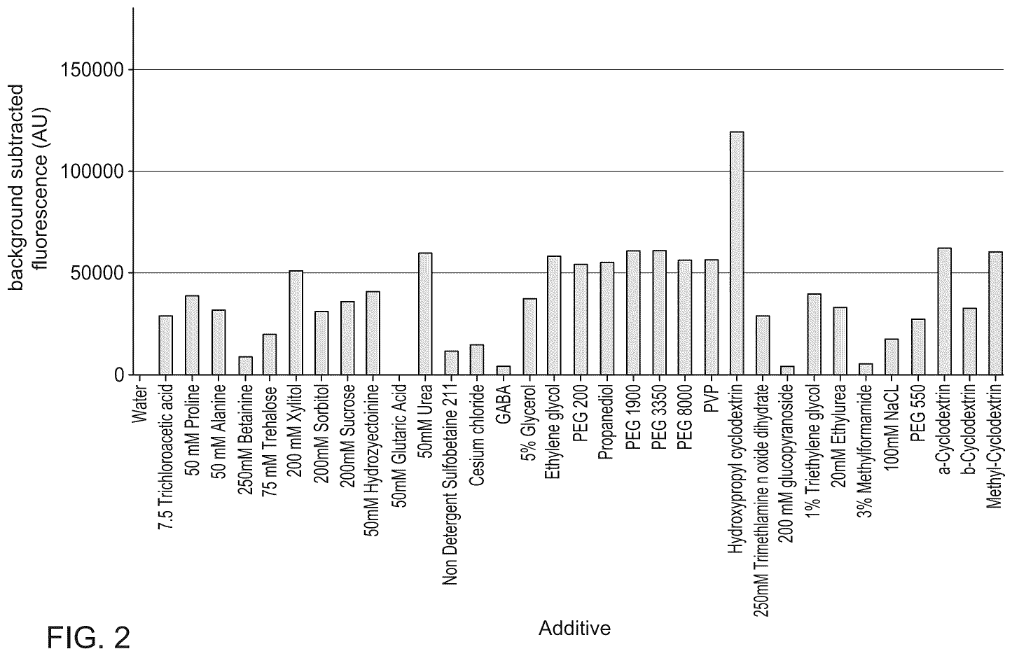

Effector proteins were tested for trans cleavage with various additives. Briefly, CasM.21526 (SEQ ID NO: 2) effector proteins were complexed with a guide nucleic acid for 15 minutes at 37° C. The 1×concentration of proteins was 40 nM and the final concentration of crRNAs was 40 nM. 5 uL of these RNPs was combined with a 15 uL mix of the following components for a total volume of 20 uL (listed at final concentration): H2B trans cleavage buffer, target nucleic acid (500 μM), 2 uL additive or water, and FQ reporter (200 nM). Reactions were carried out at 35° C. for 60 minutes. The maximum rates derived from time course experiments of the reaction with target are summarized in FIG. 2. FIG. 2 shows enhanced trans cleavage performance of CasM.21526 (SEQ ID NO: 2) in various additives in buffer H2B. CasM.21526 (SEQ ID NO: 2) trans cleavage activity appeared to be increased in the presence of simple sugars (such as xylitol, sucrose, and trehalose), amino acids (such as proline), and crowding agents (such as PEG, cyclodextrin) compared to added water under the conditions tested. Some additives, such as trichloroacetic acid, glutaric acid, and methyl-cyclodextrin exhibited non-specific activation of the fluorescent reporter when no target was added to the system under the conditions tested.

### Example 7: Trans Cleavage Activity of Effector Proteins at High Temperatures with Varying Amount of Target

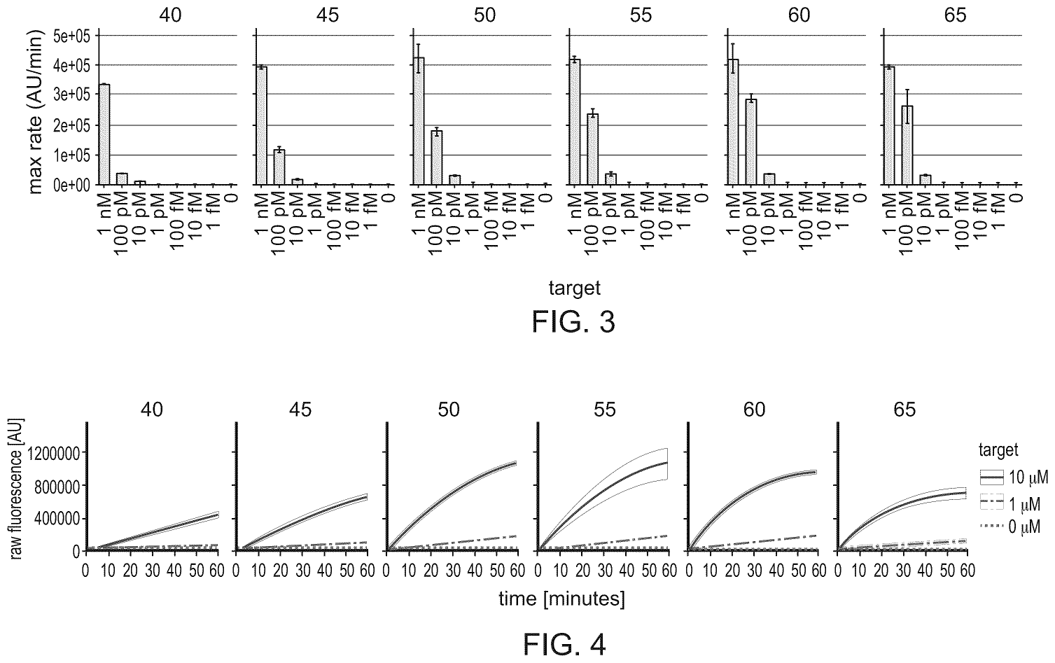

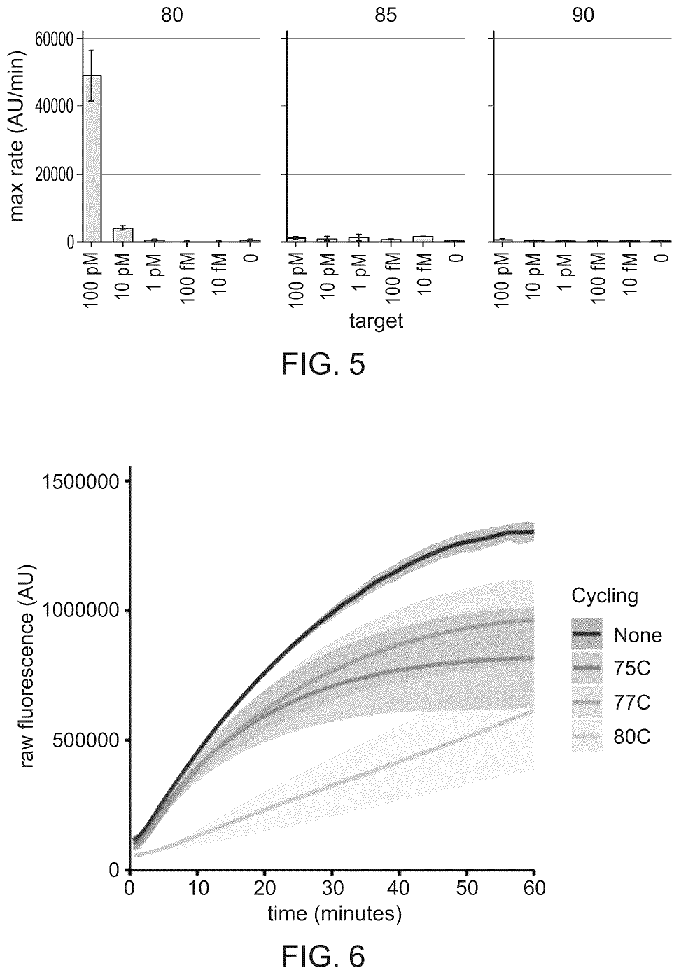

Effector proteins were tested for trans cleavage at various temperatures with varying amounts of target nucleic acid. Briefly, CasM.21526 (SEQ ID NO: 2) effector proteins were complexed with crRNA for 15 minutes at 37° C. The 1×concentration of proteins was 40 nM and the final concentration of crRNAs was 40 nM. 5 uL of these RNPs was combined with a 15 uL mix of the following components for a total volume of 20 uL (listed at final concentration): H2B trans cleavage buffer, target nucleic acid (1 nM to 0 nM), and FQ reporter (200 nM). Reactions were carried out at 40° C. to 90° C. for 60 minutes. Trans cleavage activity was detected by fluorescence signal upon cleavage of a fluorophore-quencher reporter in a DETECTR reaction. The maximum rates derived from time course experiments of the reaction with target are summarized in FIGS. 3 and 5. The trans cleavage activity over time is summarized in FIG. 4. FIG. 3 shows maximum trans cleavage rate of CasM.21526 (SEQ ID NO: 2) at 40° C. to 65° C. at various concentrations of target dsDNA. FIG. 4 shows performance over time of CasM.21526 (SEQ ID NO: 2) at 40° C. to 65° C. at various concentrations of target dsDNA. FIG. 5 shows maximum trans cleavage rate of CasM.21526 (SEQ ID NO: 2) at 80° C. to 90° C. at various concentrations of target dsDNA. CasM.21526 (SEQ ID NO: 2) exhibited trans cleavage activity over a wide range of temperatures (40° C. to 85° C.) and target concentrations (1 nM to 100 fM) under the conditions tested.

### Example 8: Thermocycling Stability of Effector Proteins

Effector proteins were tested for trans cleavage after thermocycling. Briefly, CasM.21526 (SEQ ID NO: 2) effector proteins were complexed with crRNA for 15 minutes at 37° C. The 1×concentration of proteins was 40 nM and the final concentration of crRNAs was 40 nM. 5 uL of these RNPs was combined with 15 uL of H2B trans cleavage buffer and thermocycled between 55° C. (2 seconds per cycle), 75° C. (2 seconds), and the high temperature of 75, 77, 80, 82.5, 85, 87, 90, 92.5, or 95° C. (1 second) 40 times. 15 uL of the thermocycled complex was then added to a 5 uL mix of target nucleic acid and FQ reporter (200 nM). Reactions were carried out at 60° C. for 60 minutes. Trans cleavage activity was detected by fluorescence signal upon cleavage of a fluorophore-quencher reporter in a DETECTR reaction. FIG. 6 shows performance of CasM.21526 (SEQ ID NO: 2) after thermocycling between various temperatures. CasM.21526 (SEQ ID NO: 2) retained enzymatic activity after thermocycling with a number of maximum temperatures under the conditions tested (e.g., at 75, 77, and 80° C.). There was minimal enzymatic activity after thermocycling at 82.5, 85, 87, 90, 92.5, or 95° C. maximum temperatures.

### Example 9: Threshold of Detection of Effector Proteins

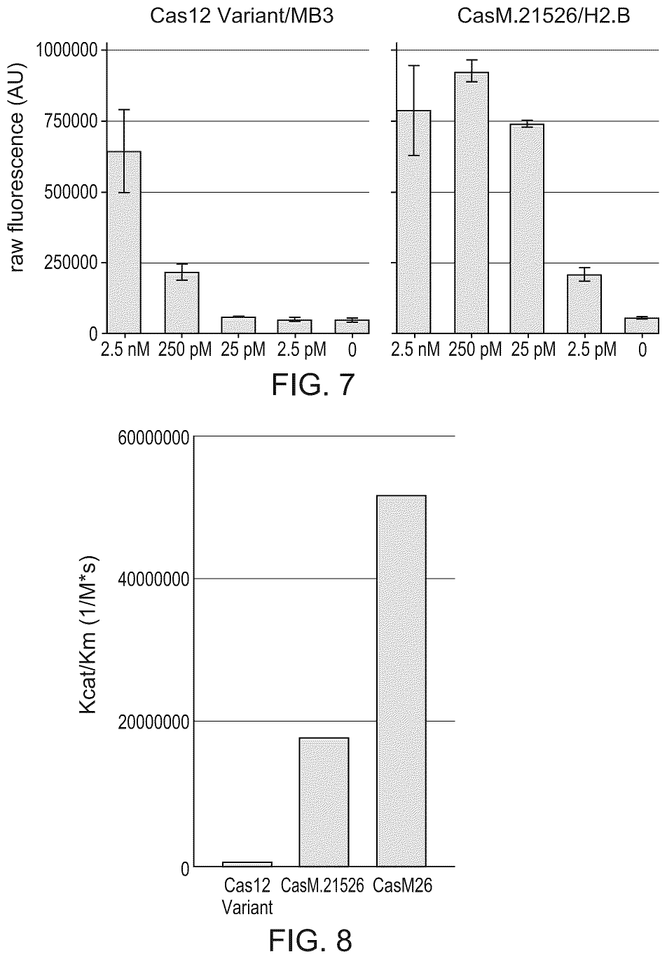

Effector proteins were tested for trans cleavage at various concentrations of target nucleic acid. Briefly, CasM.21526 (SEQ ID NO: 2) of Cas12 Variant (SEQ ID NO: 40) effector proteins were complexed with crRNA for 15 minutes at 37° C. The 1×concentration of proteins was 40 nM and the final concentration of crRNAs was 40 nM. 5 uL of these RNPs was combined with 15 uL a 15 uL mix of the following components for a total volume of 20 uL (listed at final concentration): trans cleavage buffer (H2B for CasM.21526 or MB3 for Cas12 Variant), target dsDNA (250 μM to 0 fM), and FQ reporter (200 nM). Reactions were carried out at 55° C. (for CasM.21526) or 37° C. (for Cas12 Variant) for 60 minutes. Trans cleavage activity was detected by fluorescence signal upon cleavage of a fluorophore-quencher reporter in a DETECTR reaction. FIG. 7 shows maximum trans cleavage rate for CasM.21526 (SEQ ID NO: 2) and Cas12 Variant (SEQ ID NO: 40) in preferred conditions for each enzyme. CasM.21526 (SEQ ID NO: 2) was significantly more sensitive than Cas12 Variant, with a threshold of detection of about 250 fM under the conditions tested.

### Example 10: Catalytic Efficiency of Effector Proteins

Effector proteins were tested for catalytic efficiency. Briefly, CasM.21526 (SEQ ID NO: 2), Cas12 Variant (SEQ ID NO: 40), and CasM26 (SEQ ID NO: 48) effector proteins were complexed with crRNA and target dsDNA (0.1 nM) for 30 minutes at 37° C. The 1×concentration of proteins was 40 nM and the final concentration of crRNAs was 40 nM. 5 uL of these activated RNPs was combined with a 15 uL mix of the following components for a total volume of 20 uL (listed at final concentration): trans cleavage buffer (H2B for CasM.21526, MB3 for Cas12 Variant, or MB1 for CasM26) and FQ reporter (5000 to 0 nM). Reactions were carried out at 55° C. (for CasM.21526) or 37° C. (for Cas12 Variant or CasM26) for 60 minutes and compared to unquenched reporter half (F and Q) standard curves (where known amounts of pre-cleaved reporters, one half comprising a fluorophore and one half comprising a quencher, were monitored for fluorescence). Trans cleavage activity was detected by fluorescence signal upon cleavage of a fluorophore-quencher reporter in a DETECTR reaction. Max trans cleavage rates for varying concentrations of FQ reporter were plotted and the Michaelis-Menten Equation was used to calculate Kcat and Km. FIG. 8 shows catalytic efficiency (Kcat/Km) results of CasM.21526 (SEQ ID NO: 2), Cas12 Variant (SEQ ID NO: 40), and CasM26 (SEQ ID NO: 48). CasM.21526 exhibited an enzyme turnover rate (Kcat) of 30.68 per second, a Michalis-Menten constant (Km) of 1713 nM, and a catalytic efficiency (Kcat/Km) of 1.79×10⁷ M⁻¹s⁻¹ under the conditions tested. Cas12 Variant exhibited an enzyme turnover rate (Kcat) of 0.716 per second, a Michalis-Menten constant (Km) of 1720 nM, and a catalytic efficiency (Kcat/Km) of 4.16×10⁵M⁻¹s⁻¹ under the conditions tested. CasM26 exhibited an enzyme turnover rate (Kcat) of 521.9 per second, a Michalis-Menten constant (Km) of 10105 nM, and a catalytic efficiency (Kcat/Km) of 5.16×10⁷ M⁻¹s⁻¹ under the conditions tested.

The sequence of CasM26 is SEQ ID NO: 48:

CasM26
SEQ ID NO: 48
MKVTKVGGISHKKYTSEGRLVKSESEENRIDERLSALLNMRLDMY
IKNPSSTETKENQKRIGKLKKFFSNKMVYLKDNILSLKNGKKENI
DREYSETDILESDVRDKKNFAVLKKIYLNENVNSEELEVERNDIK
KKLNKINSLKYSFEKNKANYQKINENNIEKVEGKSKRNIIYDYYR
ESAKRDAYVSNVKEAFDKLYKEEDIAKLVLEIENLTKLEKYKIRE
FYHEIIGRKNDKENFAKIIYEEIQNVNNMKELIEKVPDMSELKKS
QVFYKYYLDKEELNDKNIKYAFCHFVEIEMSQLLKNYVYKRLSNI
SNDKIKRIFEYQNLKKLIENKLLNKLDTYVRNCGKYNYYLQDGEI
ATSDFIARNRQNEAFLRNIIGVSSVAYFSLRNILETENENDITGR
MRGKTVKNNKGEEKYVSGEVDKIYNENKKNEVKENLKMFYSYDEN
MDNKNEIEDFFANIDEAISSIRHGIVHENLELEGKDIFAFKNIAP
SEISKKMFQNEINEKKLKLKIFRQLNSANVERYLEKYKILNYLKR
TRFEFVNKNIPFVPSFTKLYSRIDDLKNSLGIYWKTPKINDDNKI
KEIIDAQIYLLKNIYYGEFLNYEMSNNGNFFEISKEIIELNKNDK
RNLKTGFYKLQKFEDIQEKIPKEYLANIQSLYMINAGNQDEEEKD
TYIDFIQKIFLKGFMTYLANNGRLSLIYIGSDEETNTSLAEKKQE
FDKFLKKYEQNNNIKIPYEINEFLREIKLGNILKYTERLNMFYLI
LKLLNHKELTNLKGSLEKYQSANKEEAFSDQLELINLLNLDNNRV
TEDFELEADEIGKFLDENGNKVKDNKELKKFDINKIYEDGENIIK
HRAFYNIKKYGMLNLLEKIADKAGYKISIEELKKYSNKKNEIEKN
HKMQENLHRKYARPRKDEKFTDEDYESYKQAIENIEEYTHLKNKV
EFNELNLLQGLLLRILHRLVGYTSIWERDLRFRLKGEFPENQYIE
EIENFENKKNVKYKGGQIVEKYIKFYKELHQNDEVKINKYSSANI
KVLKQEKKDLYIRNYIAHFNYIPHAEISLLEVLENLRKLLSYDRK
LKNAVMKSVVDILKEYGFVATFKIGADKKIGIQTLESEKIVHLKN
LKKKKLMTDRNSEELCKLVKIMFEYKMEEKKSEN

### Example 11: SNP Discrimination of Effector Proteins at High Temperatures

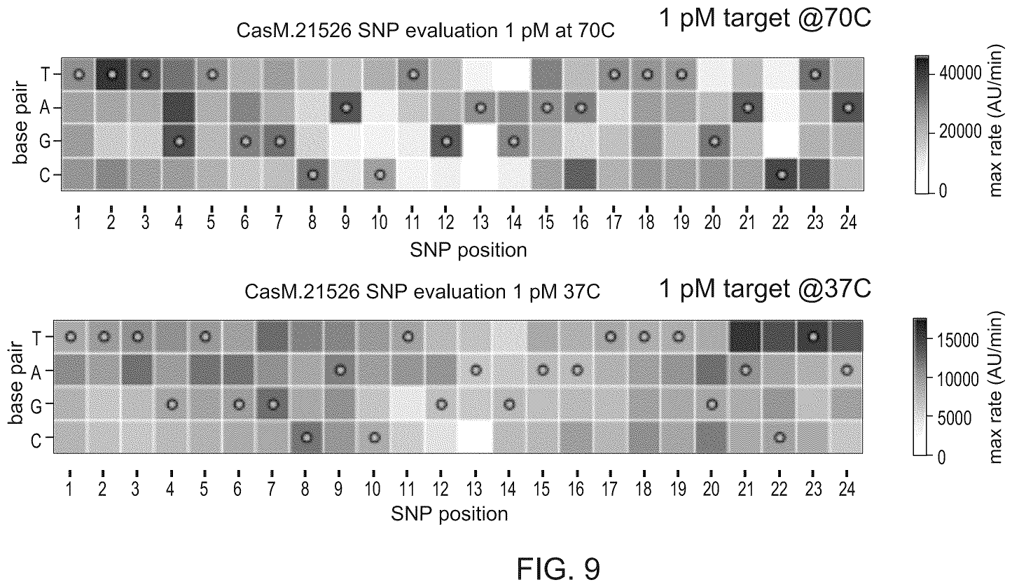

Effector proteins were tested for SNP discrimination at various temperatures. Briefly, CasM.21526 (SEQ ID NO: 2) effector proteins were complexed with crRNA for 30 minutes at 37° C. The 1× concentration of proteins was 32 nM and the final concentration of crRNAs was 40 nM. 5 uL of these RNPs was combined with a 15 uL mix of the following components for a total volume of 20 uL (listed at final concentration): H2B trans cleavage buffer, target nucleic acid (1 μM), and FQ reporter (200 nM). Reactions were carried out at 60° C. for 90 minutes. Trans cleavage activity was detected by fluorescence signal upon cleavage of a fluorophore-quencher reporter in a DETECTR reaction. Differences in the amount of trans-cleavage activity generated from a sequence with a mutation versus a sequence without a mutation were used to determine SNP sensitivity. FIG. 9 shows a heatmap of maximum trans cleavage rate of CasM.21526 (SEQ ID NO: 2) for SNP discrimination at either 70° C. or 37° C. for a variety of SNPs at 1 μM of target (normalized to wild-type (WT) trans cleavage rate, respectively). The target sequences matching the gRNA spacer sequence (SNP position 5-24) are indicated by a dot. For the PAM region (SNP position 1-4) the sequences matching the original target sequence are indicated by a dot. Results showed better SNP discrimination for CasFire at 70° C. vs at 37° C. under the conditions tested, as more dots were seen on the reaction with the highest fluorescence. There appears to be a seed region for CasM.21526 from SNP position (10-14) corresponding to positions 5-10 of the gRNA spacer sequence.

### Example 12: One-Pot DETECTR with Effector Proteins at High Temperatures

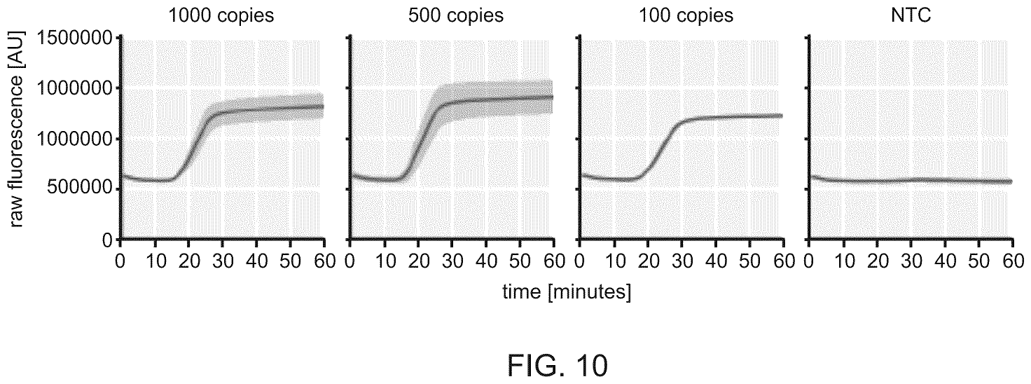

Effector proteins were tested for one-pot DETECTR reaction compatibility at high temperatures. Briefly, CasM.21526 (SEQ ID NO: 2) effector proteins were complexed with crRNA for 30 minutes at 37° C. The 1×concentration of proteins was 40 nM and the final concentration of crRNAs was 40 nM. 14.4 uL of these RNPs was combined with a 10 uL mix of the following components for a total volume of −25 uL (listed at final concentration): IB15 one port LAMP-trans cleavage buffer, SARS-CoV-2 synthetic N-gene target RNA (1000 to 0 copies, Twist Bioscience), dNTPs (1 mM), RNAse inhibitor, Bsm DNA polymerase, Warmstart RTx reverse transcriptase, SARS-CoV-2 N-gene LAMP primer mix, and FQ reporter (1000 nM). Reactions were carried out at 55° C. for 60 minutes. Trans cleavage activity was detected by fluorescence signal upon cleavage of a fluorophore-quencher reporter in the one-pot DETECTR reaction. FIG. 10 shows performance of CasM.21526 (SEQ ID NO: 2) in a one-pot DETECTR reaction at 62° C.

### Example 13: One-Pot DETECTR with Multiple Effector Proteins at High Temperatures

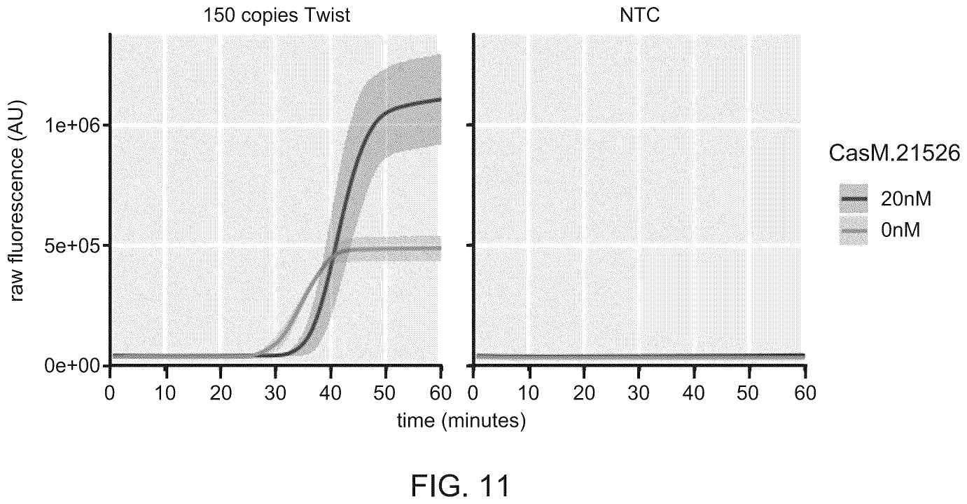

Effector proteins were mixed together and tested for one-pot DETECTR reaction compatibility at high temperatures. Briefly, CasM.21526 (SEQ ID NO: 2) and Cas14a.1 (SEQ ID NO: 41) effector proteins were complexed with crRNA for 30 minutes at 37° C. The 1×concentration of proteins was 10 nM for CasM.21526 and 40 nM for Cas14a.1 and the final concentration of crRNAs was 10 nM and 40 nM, respectively. 14.4 uL of CasM.21526 RNPs was combined with a 43.2 uL mix of the following components for a total volume of ˜57.6 uL (listed at final concentration): IB15 one port LAMP-trans cleavage buffer, SARS-CoV-2 synthetic N-gene target RNA (150 copies, Twist Bioscience), dNTPs (1 mM), RNAse inhibitor, Bsm DNA polymerase, Warmstart RTx reverse transcriptase, SARS-CoV-2 N-gene LAMP primer mix, Cas14a.1 RNPs (40 nM), and FQ reporter (1000 nM). Reactions were carried out at 55° C. for 60 minutes. Trans cleavage activity was detected by fluorescence signal upon cleavage of a fluorophore-quencher reporter in the one-pot DETECTR reaction. FIG. 11 shows performance of CasM.21526 (SEQ ID NO: 2) and Cas14a.1 (SEQ ID NO: 41) in a one-pot DETECTR reaction at 55° C. compared to Cas14a.1 alone. The sequence of Cas14a.1 is SEQ ID NO: 41:

Cas14a.1-
SEQ ID NO: 41
MAKNTITKILKLRIVRPYNSAEVEKIVADEKNNREKIALEKNKDK
VKEACSKHLKVAAYCTTQVERNACLECKARKLDDKFYQKLRGQFP
DAVEWQEISEIFRQLQKQAAEIYNQSLIELYYEIFIKGKGIANAS
SVEHYLSDVCYTRAAELFKNAAIASGLRSKIKSNFRLKELKNMKS
GLPTTKSDNFPIPLVKQKGGQYTGFEISNHNSDFIIKIPFGRWQV
KKEIDKYRPWEKFDFEQVQKSPKPISLLLSTQRRKRNKGWSKDEG
TEAEIKKVMNGDYQTSYIEVKRGSKIGEKSAWMLNLSIDVPKIDK
GVDPSIIGGIDVGVKSPLVCAINNAFSRYSISDNDLFHENKKMFA
RRRILLKKNRHKRAGHGAKNKLKPITILTEKSERFRKKLIERWAC
EIADFFIKNKVGTVQMENLESMKRKEDSYFNIRLRGFWPYAEMQN
KIEFKLKQYGIEIRKVAPNNTSKTCSKCGHLNNYENFEYRKKNKF
PHFKCEKCNFKENADYNAALNISNPKLKSTKEEP

The Cas14a.1 sgRNA used was:

(SEQ ID NO: 51)
CUUCACUGAUAAAGUGGAGAACCGCUUCACCAAAAGCUGUCCCUU
AGGGGAUUAGAACUUGAGUGAAGGUGGGCUGCUUGCAUCAGCCUA
AUGUCGAGAAGUGCUUUCUUCGGAAAGUAACCCUCGAAACAAAUU
CAUUUGAAAGAAUGAAGGAAUGCAACCCCCCAGCGCUUCAGCGUU
C
(SARS-CoV-2 N-gene, PAM: TTTG)

### Example 14: Lateral Flow-based DETECTR with Effector Proteins at High Temperatures

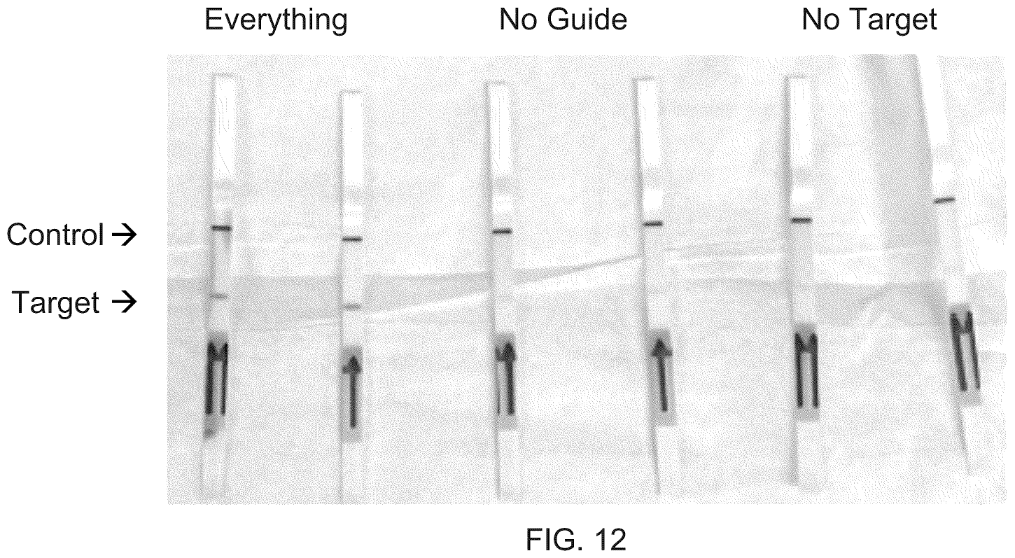

Effector proteins were tested for lateral flow-based DETECTR reaction compatibility at high temperatures. Briefly, a 2:1 ratio mixture of unfunctionalized PEG (MW=600 monomers) and PEG-diacrylate (MW=700 monomers) were mixed together with a photoinitiator (2-Hydroxy-2-methylpropiophenone (Darocur 1173)) and 100 μM of Acrydite-modified “Rep172” reporter (/5Acryd/TTT TTT TTT TTT TTT TTT TT (SEQ ID NO: 49)/i6-FAMK//3Bio/). The mixture was exposed to UV light (365 nm, 200 ms) under a photomask to generate circular cross-section rods of hydrogel containing immobilized reporters. CasM.21526 (SEQ ID NO: 2) effector proteins were complexed with a guide nucleic acid or no guide nucleic acids for 30 minutes at 37° C. 5 uL of these RNPs was combined with 2 uL of target dsDNA (1 nM final concentration) or no target control (“no target”), 2 uL of reporter-immobilized hydrogels, and 11 uL of H2B buffer for a total volume of 20 uL. Reactions were carried out at 57° C. for 10 minutes. Trans cleavage activity was detected with lateral flow assay strips. The supernatant for each reaction was then applied to the sample pad of a lateral flow assay strip containing anti-FITC conjugate particles (colloidal gold). If trans cleavage occurred, the supernatant contained cleaved FAM-biotin-labeled reporter molecules which bound to an anti-biotin (e.g., streptavidin) target line on the lateral flow strip. The anti-FITC conjugate particles bound the FAM moiety on the reporter molecules and a target band appeared on lateral flow strips at the anti-biotin target line. If trans cleavage did not occur (as in NTC or no guide RNA reactions), the supernatant did not contain any FAM-biotin-labeled molecules, and nothing bound to the anti-biotin target line. The lateral flow assay strip also contained an anti-IgG flow control line, downstream of the anti-biotin target line, which bound to the anti-FITC moiety of the conjugate particles to confirm that the lateral flow assay functioned properly. FIG. 12 shows lateral flow assay results after DETECTR reactions using CasM.21526 (SEQ ID NO: 2) with target and guide present (“everything”), with target but no guideRNA present (“no guide”), and without target (“no target”). Strong signals were seen in both positive sample replicates (“everything”), with minimal background appearing in the “no guide” and “no target” replicates at the target line.

### Example 15: Guide-Pooled DETECTR with Effector Proteins at High Temperatures

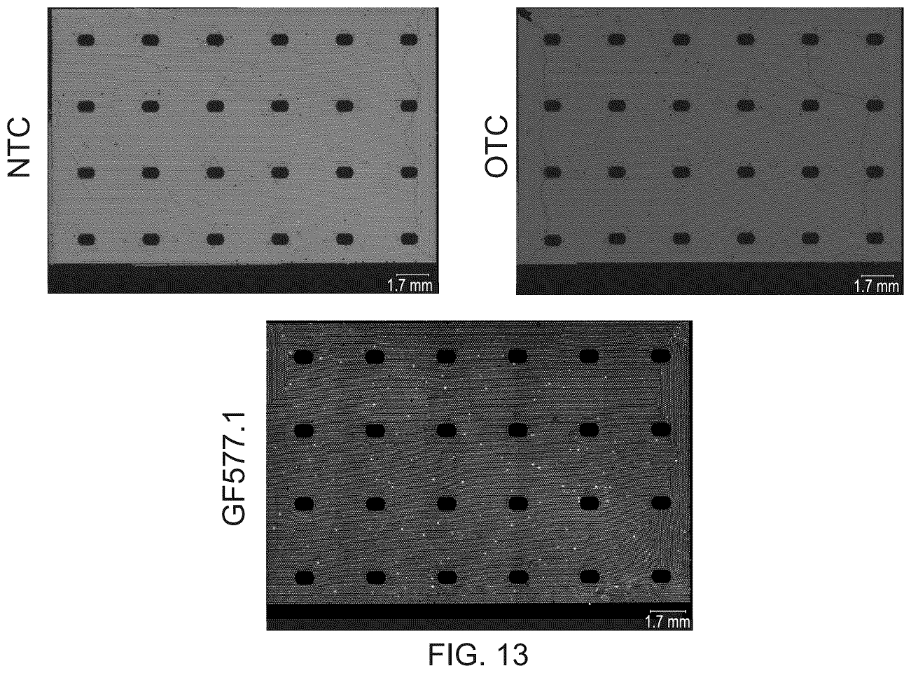

Effector proteins were tested for guide-pooled DETECTR reaction compatibility at high temperatures. Briefly, CasM.21526 (SEQ ID NO: 2) effector proteins were complexed with a 4×crRNA pool (4 guide pool) or an off-target guide (“OTC”) for 30 minutes at 37° C. The 1×concentration of proteins was 40 nM and the final concentration of crRNAs was 62.5 nM. 15 uL of these RNPs was combined with a 45 uL mix of the following components for a total volume of 60 uL (listed at final concentration): 1×H2B trans cleavage buffer, 6,250 copies/reaction GF577.1 dsDNA target (“GF577.1” or “OTC”) or water (“NTC”), 100 nM fluorescein, and “rep133” reporter at 1.5 nM. 25 uL of the DETECTR mixture was then partitioned into each chamber of a dPCR chip at 40° C. (no amplification step was performed). Reactions were carried out at 60° C. for 90 minutes. Trans cleavage activity was detected by fluorescence signal upon cleavage of the fluorophore-quencher reporter in the DETECTR reaction. FIG. 13 shows fluorescent images of CasM.21526 (SEQ ID NO: 2) dPCR chips after the guide-pooled DETECTR reaction was completed. Fluorescence signal was observed in the on-target condition (GF5771.1) but not in the off-target control (OTC) or no template control (NTC) for the conditions tested, thus CasM.21526 was able to directly detect the target DNA in the guide-pooled DETECTR reaction.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising an effector protein and an engineered guide nucleic acid, wherein the effector protein comprises an amino acid sequence that is at least 75% identical to SEQ ID NO: 2 and wherein the engineered guide nucleic acid comprises a nucleobase sequence that is at least 95% identical to GAAUUUCUACUAUUGUAGAU (SEO ID NO: 55) or UAAUUUCUACUAAGUGUAGAU (SEO ID NO: 56).

2. The composition of claim 1, wherein the effector protein does not comprise the amino acid sequence MEEK (SEO ID NO: 47).

3. The composition of claim 1, wherein the engineered guide nucleic acid comprises a nucleobase sequence that is at least 75% identical to any one of SEQ ID NOs: 19-21 or 52-54.

4. The composition of claim 1, wherein the effector protein recognizes a protospacer adjacent motif (PAM) sequence present in a target nucleic acid.

5. The composition of claim 4, wherein the PAM sequence is YYN, wherein N is an adenine (A), a guanine (G), a cytosine (C), or a thymine (T); and wherein Y is a C or T.

6. The composition of claim 1, wherein the composition does not comprise Thermostable Inorganic Pyrophosphatase (TIPP).

7. The composition of claim 1, wherein the composition further comprises Mg²⁺.

8. The composition of claim 1, wherein the composition has a pH within a range of from about 8.0 to about 9.0.

9. The composition of claim 1, wherein the composition further comprises a reporter nucleic acid.

10. The composition of claim 1, wherein the composition has a temperature of at least 45° C.

11. (canceled)

12. The composition of claim 1, wherein the effector protein has a threshold of detection of less than 250 pM of a target nucleic acid at a temperature within a range of from about 45° C. to about 80° C.

13-15. (canceled)

16. A composition comprising an effector protein and an engineered guide nucleic acid, wherein the effector protein has catalytic efficiency of at least about 1.7×10⁷ M⁻¹s⁻¹ at a temperature within a range of from about 45° C. to about 80° C.

17. A composition comprising an effector protein and an engineered guide nucleic acid, wherein the effector protein provides higher transcollateral cleavage activity at 70° C. than at 37° C. when a target nucleic acid comprises a single nucleotide polymorphism (SNP).

18-20. (canceled)

21. A system for detecting a target nucleic acid, comprising the composition of claim 1 in a solution, wherein the solution comprises at least one of a buffering agent, a salt, a crowding agent, a detergent, a reducing agent, a competitor, and a detection agent.

22. The system of claim 21, wherein the pH of the solution is at least about 6.0.

23. The system of claim 21, wherein the salt is selected from the group consisting of a magnesium salt, a potassium salt, a sodium salt, and a calcium salt.

24. (canceled)

25. The system of claim 21, wherein the detection reagent is selected from a reporter nucleic acid, a detection moiety, an additional effector protein, an enzyme, or a combination thereof.

26. (canceled)

27. The system of claim 25, wherein the reporter nucleic acid comprises a fluorophore, a quencher, or a combination thereof.

28. The system of claim 26, wherein the reporter nucleic acid is in the form of single stranded deoxyribonucleic acid (DNA).

29. The system of claim 21, comprising at least one amplification reagent for amplifying the target nucleic acid.

30-33. (canceled)

34. A method of detecting a target nucleic acid in a sample, comprising:
a. contacting the sample with:
i. an effector protein, wherein the effector protein comprises an amino acid sequence at least 70% identical to SEQ ID NO: 2,
ii. an engineered guide nucleic acid, and
iii. a detection reagent that is cleaved in the presence of the effector protein, the engineered guide nucleic acid, and a target nucleic acid, wherein the target nucleic acid comprises a PAM sequence selected from the group consisting of NNNNNYN (SEQ ID NO: 42), NNNNYYN (SEQ ID NO: 43), NNNNYTN (SEQ ID NO: 44), and NNNNYNN (SEQ ID NO: 45), wherein T is thymine (T), wherein N is adenine (A), guanine (G), cytosine (C), or T, and wherein Y is a C or T; and
b. detecting a signal produced by cleavage of the detection reagent, thereby detecting the target nucleic acid in the sample.

35. The method of claim 34, wherein the method comprises amplifying the target nucleic acid.

36. The method of claim 35, wherein amplifying is performed before contacting.

37. The method of claim 35, wherein amplifying is performed during contacting.

38. The method of claim 34, wherein detecting is performed at a temperature of at least about 40° C.

39-148. (canceled)
